(19) United States
(12) Patent Application Publication
(10) Pub. No.: US 2021/0010031 A1
(43) Pub. Date: Jan. 14, 2021

(54) LENTIVIRAL VECTORS FOR HIGH-TITER TRANSDUCTION OF PRIMARY HUMAN CELLS

(71) Applicant: University of Massachusetts, Boston, MA (US)

(72) Inventors: Jeremy Luban, Newton, MA (US); Kyusik Kim, Allston, MA (US); Sean Matthew McCauley, North Grafton, MA (US)

(73) Assignee: University of Massachusetts, Boston, MA (US)

(21) Appl. No.: 17/042,981

(22) PCT Filed: Mar. 29, 2019
(86) PCT No.: PCT/US19/24905

§ 371 Date: Sep. 29, 2020

Related U.S. Application Data
(60) Provisional application No. 62/650,973, filed on Mar. 30, 2018.
(60) Provisional application No. 62/650,977, filed on Mar. 30, 2018.

(51) Int. Cl. C12N 15/86; A61K 35/76; C12N 9/22

(52) U.S. Cl. CPC C12N 15/86; A61K 35/76; C12N 2800/80; C12N 2740/16043; C12N 9/22

(57) ABSTRACT

Aspects of the disclosure relate to packagable RNA constructs with minimal intervening viral sequences. These constructs can be used to generate lentiviral viruses encoding large genes capable of transducing primary human cells.

### RELATED APPLICATIONS

This Application is a national stage filing under 35 U.S.C. § 371 of international PCT application, PCT/US2019/024905, filed Mar. 29, 2019, which claims priority under 35 U.S.C. § 119(e) to U.S. provisional patent application, U.S. Ser. No. 62/650,973, filed Mar. 30, 2018 and U.S. provisional patent application, U.S. Ser. No. 62/650,977, filed Mar. 30, 2018, the entire contents of each of which are incorporated herein by reference.

### BACKGROUND

Primary human cells, particularly immune cells, are difficult to transduce with lentiviral vectors, particularly as the size of the gene encoded in by the nucleic acid increases above 3,000 nucleotides. With the advent of Clustered Regularly Interspaced Short Palindromic Repeat (CRISPR) gene editing technology, the need to package the large Cas nuclease gene, which is anywhere from 6,000-8,000 nucleotides for commonly used genes, has become relevant to the ability to edit the genome.

### SUMMARY

In some aspects, the disclosure relates to nucleic acid constructs (e.g., plasmids) encoding packagable vector RNAs that are capable of delivering large heterologous nucleic acid inserts (e.g., transgenes) to cells. The disclosure is based, in part, on nucleic acid constructs engineered to contain minimal intervening sequences (typically of viral origin), which in some embodiments facilitates the incorporation of relatively large inserts into the constructs. In some embodiments, nucleic acid constructs of the disclosure advantageously allow for the packaging and production of high titers of viral particles containing the vector RNAs. In some embodiments, viral particles comprising the vector RNAs can achieve relatively high levels of cellular transduction, including in primary cells. In some embodiments, vectors described by the disclosure are useful for delivery of large genes, for example Cas9 gene, to cells that have historically been difficult to transfect, such as primary human dendritic cells.

In some aspects, the disclosure provides a construct comprising a promoter operably linked to a nucleic acid encoding a packagable vector RNA. In some embodiments, the packagable vector RNA comprises 5′- and 3′-terminal repeats (TRs) that flank a nucleocapsid protein packaging target site, a heterologous nucleic acid insert, and minimal intervening viral sequences. In some embodiments, these packable vector RNAs are lentiviral-based RNAs.

In some embodiments, the TRs further flank a REV protein response element (RRE) and a polypurine tract. In some embodiments, the TRs further flank a sequence encoding a GAG protein.

In some embodiments, one or both of the TRs is a lentiviral long terminal repeat. In some embodiments, one or both of the 5-′ and 3′-terminal repeats is a truncated long terminal repeat that comprises an R-element that directs reverse transcription and an integrase subelement that directs integration.

In some embodiments, minimal intervening viral sequences have a total length of up to 350 base pairs.

In some aspects, the disclosure provides a nucleic acid comprising a heterologous nucleic acid insert flanked by TRs, in which between a first TR (e.g., a 5′-TR or a 3′-TR) and the heterologous nucleic acid sequence are present packaging and nuclear export sequences and minimal intervening viral sequences.

In some embodiments, a promoter is located before the 5′-TR. In some embodiments, the promoter is a constitutive promoter. In some embodiments, the constitutive promoter is CMV or SV40.

In some embodiments, there is an internal promoter operably linked to the heterologous nucleic acid insert located between the nucleocapsid protein packaging target site and the second TR. In some embodiments, the internal promoter is a spleen focus-forming virus (SFFV) promoter.

In some embodiments, the 5′-TR is a RNA pol II promoter and comprises a repeat region and a U5 region. In some embodiments, the 3′-TR is a transcription termination and comprises a repeat region and a U3 region.

In some embodiments, the packaging sequences comprise a psi (ψ) sequence and a polypurine tract sequence. In some embodiments, the order of the packaging sequences is w sequence followed by polypurine tract.

In some embodiments, the nuclear export sequence comprises a Rev Response Element (RRE). In some embodiments, the RRE is located between the ψ sequence and the polypurine tract sequence.

In some embodiments, a packagable nucleic acid (e.g., packagable vector RNA) size is 1,900 bases, plus the size of the heterologous insert (e.g., 1900 bases without the heterologous insert sequence).

In some embodiments, the heterologous nucleic acid insert is engineered to express a protein or a functional RNA.

In some aspects, the disclosure provides a plasmid that comprises the packagable vector RNA construct with minimal intervening viral sequences. In some embodiments, the disclosure provides a construct comprising a nucleic acid comprising a heterologous nucleic acid insert flanked by TRs, wherein between a first TR and the heterologous nucleic acid sequence are present packaging and nuclear export sequences and minimal intervening viral sequences.

In some aspects, the disclosure provides a method of delivering to a cell a plasmid comprising the packagable vector RNA construct with minimal intervening viral sequences. In some embodiments, the disclosure provides a method of delivering to a cell a plasmid comprising a nucleic acid comprising a heterologous nucleic acid insert flanked by TRs, in which between a first TR and the heterologous nucleic acid sequence are present packaging and nuclear export sequences and minimal intervening viral sequences.

In some aspects, the disclosure provides a host cell comprising a packagable vector RNA construct with minimal intervening viral sequences. In some embodiments, the disclosure provides a host cell comprising a nucleic acid comprising a heterologous nucleic acid insert flanked by TRs, wherein between a first TR and the heterologous nucleic acid sequence are present packaging and nuclear export sequences and minimal intervening viral sequences.

In some embodiments, the host cells further comprises a RNA polymerase that selectively binds to the 5′-TR of the nucleic acid. In some embodiments, the host cell further comprises plasmids encoding nucleic acid sequences which facilitate the packaging and enveloping of the transcribed nucleic acid. In some embodiments, the envelope sequence is vesicular stomatitis virus G glycoprotein (VSVG). In some embodiments, the packaging sequences encode GAG, Pol, and Rev proteins.

In some aspects, the disclosure provides a transcribed nucleic acid encoding a packagable vector RNA construct with minimal intervening viral sequences. In some embodiments, the disclosure provides a transcribed nucleic acid encoding a heterologous nucleic acid insert flanked by TRs, wherein between the first TR and the heterologous nucleic acid sequence, there are sequences that aid in the packaging and nuclear export of the transcribed nucleic acid and minimal intervening viral sequences.

In some embodiments, the disclosure provides a host cell comprising the transcribed nucleic acid encoding a packagable vector RNA construct with minimal intervening viral sequences.

In some aspects, the disclosure provides a host cell comprising viral particles, wherein the transcribed nucleic acid encoding a packagable vector RNA construct with minimal intervening viral sequences is within the viral particles. In some embodiments, the disclosure provides a method for infecting a host cell with the viral particles. In some embodiments, the disclosure provides a method for infecting a subject with the viral particles.

In some aspects, the disclosure provides a composition comprising a plurality of nucleic acids. In some embodiments, the composition comprises a plurality of nucleic acids and a pharmaceutically acceptable carrier.

In some aspects the disclosure provides a construct comprising a promoter operably linked to a nucleic acid encoding a packagable vector RNA, the packagable vector RNA comprising 5′- and 3′-terminal repeats (TRs) that flank a nucleocapsid protein packaging target site, a heterologous nucleic acid insert, and minimal intervening viral sequences, in which the heterologous nucleic acid insert encodes a shRNA sequence. In some embodiments, there is a selectable marker gene upstream of the shRNA sequence. In some embodiments, there is a reporter gene upstream of the shRNA sequence.

In some aspects the disclosure provides a construct comprising a promoter operably linked to a nucleic acid encoding a packagable vector RNA, the packagable vector RNA comprising 5′- and 3′-terminal repeats (TRs) that flank a nucleocapsid protein packaging target site, a heterologous nucleic acid insert, and minimal intervening viral sequences, wherein the heterologous nucleic acid insert encodes a Cas nuclease. In some embodiments, the Cas nuclease is Cas9 nuclease. In some embodiments, the Cas9 nuclease is from Streptococcus pyogenes, Neisseria meningitides, or Campylobacter jejuni.

In some aspects, the disclosure provides a plasmid that carries a construct comprising a promoter operably linked to a nucleic acid encoding a packagable vector RNA, the packagable vector RNA comprising 5′- and 3′-terminal repeats (TRs) that flank a nucleocapsid protein packaging target site, a heterologous nucleic acid insert, and minimal intervening viral sequences, wherein the heterologous nucleic acid sequence optionally encodes a shRNA or a Cas protein as outlined above.

In some embodiments, the disclosure provides a host cell comprising the transcribed nucleic acid encoding a packagable vector RNA construct with minimal intervening viral sequences, wherein the heterologous insert encodes either a shRNA or a Cas protein as outlined above.

In some embodiments, the disclosure provides a method of delivering a plasmid that carries a construct comprising a promoter operably linked to a nucleic acid encoding a packagable vector RNA, the packagable vector RNA comprising 5′- and 3′-terminal repeats (TRs) that flank a nucleocapsid protein packaging target site, a heterologous nucleic acid insert, and minimal intervening viral sequences, wherein the heterologous nucleic acid sequence optionally encodes a shRNA or a Cas protein to a host cell. In some embodiments, the host cell further comprises an RNA polymerase that selectively binds to the 5′-TR of the nucleic acid. In some embodiments the host cell further comprises plasmids encoding nucleic acid sequences that facilitate packaging of the transcribed nucleic acid. In some embodiments, the envelope sequence is vesicular stomatitis virus G glycoprotein (VSVG). In some embodiments, the packaging sequences encode GAG, Pol, and Rev proteins.

In some aspects, the disclosure provides a host cell comprising viral particles wherein the transcribed nucleic acid construct comprising a promoter operably linked to a nucleic acid encoding a packagable vector RNA, the packagable vector RNA comprising 5′- and 3′-terminal repeats (TRs) that flank a nucleocapsid protein packaging target site, a heterologous nucleic acid insert, and minimal intervening viral sequences, wherein the heterologous nucleic acid sequence optionally encodes a shRNA or a Cas protein is within the viral particles. In some embodiments, the disclosure provides a method for infecting a host cell with the viral particles. In some embodiments, the disclosure provides a method for infecting a subject with the viral particles.

In some aspects, the disclosure provides a composition comprising a plurality of nucleic acids comprising a promoter operably linked to a nucleic acid encoding a packagable vector RNA, the packagable vector RNA comprising 5′- and 3′-terminal repeats (TRs) that flank a nucleocapsid protein packaging target site, a heterologous nucleic acid insert, and minimal intervening viral sequences, wherein the heterologous nucleic acid sequence optionally encodes a shRNA or a Cas protein. In some embodiments, the composition comprises a plurality of nucleic acids and a pharmaceutically acceptable carrier.

In some aspects, the host cell is a primary human cell. In some embodiments, the host cell is a human primary dendritic cell.

In some aspects, the disclosure provides a method for efficient gene knockdown, the method comprising infecting target cells with viral particles enclosing nucleic acid construct comprising a promoter operably linked to a nucleic acid encoding a packagable vector RNA, the packagable vector RNA comprising 5′- and 3′-terminal repeats (TRs) that flank a nucleocapsid protein packaging target site, a heterologous nucleic acid insert, and minimal intervening viral sequences, wherein the heterologous nucleic acid sequence optionally encodes a shRNA or a Cas protein. In some embodiments, the target cells are primary human cells. In some embodiments, the primary human cells are dendritic cells.

In some aspects, the disclosure provides a kit containing a plasmid comprising a nucleic acid construct comprising a promoter operably linked to a nucleic acid encoding a packagable vector RNA, the packagable vector RNA comprising 5′- and 3′-terminal repeats (TRs) that flank a nucleocapsid protein packaging target site, a heterologous nucleic acid insert, and minimal intervening viral sequences.

In some aspects, the disclosure provides a construct comprising a packagable vector RNA as depicted in FIG. 15.

Aspects of the disclosure relate to lentivector constructs comprising a promoter operably linked to a nucleic acid encoding a packagable vector RNA, the packagable vector RNA comprising 5′- and 3′-terminal repeat (TRs) that flank a heterologous nucleic acid insert with minimal intervening viral sequences. In some embodiments, the heterologous nucleic acid insert encodes an miRNA based shRNA.

In some aspects, the disclosure relates to a plasmid listed in Table 2. In some embodiments, the disclosure relates to a construct comprising a sequence as set forth in SEQ ID NO: 10. In some embodiments, the disclosure relates to a construct comprising a sequence as set forth in SEQ ID NO: 11, encoding an miRNA based shRNA that is engineered to target a gene listed in Table 2. In some embodiments, the disclosure relates to a construct comprising a sequence as set forth in SEQ ID NO: 1, encoding an miRNA based shRNA that is engineered to target AGO1, AGO2, AGO3, DNMT3A, HDAC1, HP1, SUV39H1, SUV39H2, PIWIL2, TRIM28, SETDB1, FAM208A, MPHOSPH8, PPHLN1, or MORC2.

### BRIEF DESCRIPTION OF DRAWINGS

FIG. 1 is a schematic diagram of the lentiviral vector plasmids, showing only the vector elements.

FIG. 2 shows vector development from the standard vector to the first generation.

FIG. 3 shows the knockdown construct for testing the first generation vector.

FIG. 4 shows expression levels (fold change and fold reduction) with a regular lentiviral vector versus a first generation lentiviral vector.

FIG. 5 shows a schematic of single-cell sequencing with shRNA library screening as a further application of the vector development.

FIG. 6 shows further development of the lentiviral vector from first to second generation.

FIG. 7 compares the structures of the regular lentiviral vector and the second generation vector.

FIG. 8 shows the Cas9 construct for testing first and second generation vectors on human primary dendritic cells.

FIG. 9 shows transduction efficiency of the SpyCas9 construct.

FIG. 10 shows examples of Cas9 constructs.

FIG. 11 shows transduction efficiency of different Cas9 constructs.

FIG. 12 shows a schematic of the test of whether transduced Cas9s disrupt target gene expression in human primary dendritic cells.

FIG. 13 shows disruption of cell surface levels of the protein encoded by the target gene, DC-SIGN, with SpyCas9 transduction.

FIG. 14 shows the generations of packagable viral RNA constructs, including future planned generations wherein even more viral sequence has been eliminated.

FIG. 15 shows a map of one embodiment of a pTL packagable viral RNA construct, with the lengths of intervening viral sequences labeled.

FIGS. 16A to 16E show diverse primate immunodeficiency virus vpx and vpr orthologues activate provirus transcription, whether delivered before, during, or after reporter provirus integration. FIG. 16A shows a schematic of experimental protocol in FIG. 16B. FIG. 16B shows a flow cytometry plot showing percent GFP⁺ Jurkat cells after sequential transduction with the indicated lentivectors, followed by exposure to the indicated VLPs. FIGS. 16C and 16D show histograms of flow cytometry signal in Jurkat cells transduced with gfp-reporter virus, and either exposed to the indicated VLPs (FIG. 16C), or transduced with the indicated vectors (FIG. 16D). FIG. 16E shows a phylogenetic tree showing evolutionary relationship of Vpx and Vpr proteins. The transactivation activity of Jurkat reporter lines, tested as in FIG. 16D, and human SAMHD1 degradation activity, are indicated. Ø indicates Vprs that were too toxic (G2 arrest) for assessment. All data shown is representative of at least three biological replicates.

FIGS. 17A to 17H show Vpx activates provirus transcription by degrading HUSH complex components. FIG. 17A shows Jurkat cells transduced with shRNA-puro^{R} vectors targeting the indicated genes were selected with puromycin, transduced with Lenti 2-Avpx, and analyzed 5 days later. Plot depicts GFP signal in knockdown lines relative to Jurkats bearing SIV_{MAC}251 vpx (mean±S.E.M., n=3 shRNA target sites). *, P<0.05 as determined by 1-way ANOVA with Dunnett post-test, relative to luciferase knockdown control. FIG. 17B shows Jurkat cells were transduced with the indicated shRNA-puro^{R} vectors and selected with puromycin. Resistant cells were transduced with vpx⁺ or Δvpx Lenti 2 vector, and analyzed for GFP expression 7 days later. FIG. 17C shows immunoblot analysis for components of the HUSH complex in Jurkat cells expressing shRNA constructs used in FIG. 17B. FIG. 17D shows CD4⁺ T cells were activated for 3 days with PHA and then transduced and assayed as in FIG. 17B. FIG. 17E shows immunoblot analysis of Jurkat lines transduced to express vpx from SIV_{MAC}251, SIV_{RCM}NG411, SIV_{MND2}5440, or control. FIG. 17F shows levels of HUSH components in FIG. 17E shown as shRNA treated condition relative to control. FIG. 17G shows FAM208A, DCAF1, and Actin immunoblot of Jurkat cells transduced with DCAF1 shRNA-puro^{R} vector or control, that were treated with Vpx⁺ or ΔVpx VLPs for 18 hrs. FIG. 17H shows HEK293 cells were co-transfected with HA-FAM208A and the indicated FLAG-Vpx constructs. 18 hrs after transfection, cells were either exposed to proteasome inhibitor PR171 or left untreated. 8 hrs after inhibitor treatment cells were lysed, FLAG-Vpx was immunoprecipitated, and immunoblotted for FLAG-Vpx and HA-FAM208A. Immunoblotting of input lysates are shown below.

FIGS. 18A to 18F show the HIV-1 LTR is activated by Vpx or disruption of FAM208A. FIG. 18A shows a schematic of the HIV-1 minigenome integrated in the J-Lat A1 line. FIG. 18B shows J-Lat A1 cells were transduced with Lenti 1 encoding SIV_{MAC}251 vpx or Δvpx control, or with lentivectors expressing shRNA targeting FAM208A or luciferase control. Transduced cells were selected with puromycin, and activated for 24 hrs with 10 ng/ml of TNFα. Representative GFP signal by flow is shown. FIG. 18C shows quantification of results from FIG. 18B and additional replicates (mean±S.E.M., n=3 independent experiments). *, P<0.02 FIG. 18D: Schematic of the LTR-gfp provirus used to analyze HIV-1 LTR driven gfp expression in pools of cells. FIG. 18E shows Jurkat cells transduced with LTR-gfp were kept in culture for 4 weeks and then transduced and assessed by flow cytometry, as in FIG. 18B. FIG. 18F shows quantification of results from FIG. 18E (mean±S.E.M., n=4 independent experiments) *, P<0.02

FIGS. 19A to 19E show Vpx counteracts FAM208A restriction of HIV-1, SIV_{MAC}239, or HIV-2_{GH}, during spreading infection in CD4⁺ T cells. FIGS. 19A-19B show replication of HIV-1-ZsGreen in Jurkat cells transduced with SIV_{MAC}251 vpx or control (FIG. 19A), or with lentivectors expressing shRNA targeting FAM208A or Luc control (FIG. 19B). Replication kinetics was measured by flow cytometry for ZsGreen⁺ cells. FIGS. 19C-19E show spreading infection of HIV-1-ZsGreen (FIG. 19C), SIV_{MAC}239 or SIVMAc239Δvpx (FIG. 19D), and HIV-2_{GH} or HIV-2_{GH} Δvpx virus in CEMx174 cells transduced with FAM208A or Luc control shRNA. Spread of HIV-1-ZsGreen was assessed by flow cytometry, while spread of SIV_{mac}239 (FIG. 19B) and HIV-2_{GH} (FIG. 19C) was assessed by measuring the accumulation of reverse transcriptase (RT) activity in the supernatant. All data is representative of three repeat experiments.

FIGS. 20A to 20F show transcriptional activation of lentivector reporter genes by vpx and vpr. FIG. 20A shows a schematic of vpx⁺ and no vpx versions of Lenti 1 and Lenti 2 vectors used in FIGS. 16A to 16E and 17A to 17H. FIG. 20B shows representative live, singlet, lymphoid, GFP flow cytometry gating strategy. FIG. 20C shows quantification of results from FIG. 20B. Jurkat-vpx or Jurkat-puro^{R} transduced with Lenti-2-vpx, or Lenti-2-no vpx, were treated with Vpx⁺ VLPs, ΔVpx VLPs, or no VLPs, and analyzed three days later. MFI was normalized for each group of VLP treated cells to untreated samples; mean±S.E.M., n=3 independent experiments. Significance was determined by 1-way ANOVA with Dunnett post-test comparing treated to untreated samples in each group. *, P<0.016. FIG. 20D: Representative qPCR analysis of gfp expression after Lenti-gfp-blasti^{R} cells were transduced with SIV_{MAC}251 Vpx or empty vectors (mean±S.E.M., n=3 replicates) *, P<0.02. FIG. 20E shows Jurkat cells transduced with Lenti-gfp-blasti^{R} with GFP driven by EFla or TK promoters and Blasti^{R} driven by CypA promoter. 3 days after selection cells were transduced with SIV_{MAC}251 Vpx (white) or control puro^{R} (red) vectors and selected with blasticidin. Untransduced cells are shown in grey. FIG. 20F shows transactivation of Lenti-gfp-blasti^{R} reporter cells by the indicated vpx and vpr expression vectors. Line indicates 4-fold transactivation, which was used as a cutoff for activity.

FIGS. 21A to 21C show HUSH components inhibit provirus expression in primary CD4+ T cells; Vpx and Vpr from multiple lentiviral species deplete FAM208A. FIG. 21A shows quantification of results from FIG. 17D. CD4⁺ T cells were positively selected with magnetic beads, activated for 3 days with PHA, transduced with the indicated shRNA-puro^{R} knockdown or control vectors, and selected with puromycin. Cells were then transduced with a lenti-gfp vector in the absence of vpx, and analyzed for GFP expression 7 days later (mean±S.E.M., n=3 donors). FIG. 21B shows immunoblotting for FAM208A and Actin using lysate from Jurkat cells stably transduced with lentivectors producing the indicated Vpx proteins. FIG. 21C shows immunoblotting for FAM208A, FLAG-Vpx, and FLAG-Vpr in Jurkat cells stably transduced with lentivectors expressing the indicated 3×FLAG tagged Vpx and Vpr constructs.

FIGS. 22A to 22C shows expression from the HIV-1 LTR is activated by diverse Vpx and Vpr proteins. FIG. 22A shows J-Lat A1 cells transduced with Lenti 1 encoding Vpx from SIV_{MAC}251, SIV_{RCM}02CM8081, or SIV_{MND}25440, Vpr from SIV_{MND1}GB1, or SIV_{AGM}TAN1, or control no vpx Lenti 1. Transduced cells were selected with puromycin, activated for 24 hrs with 10 ng/ml of TNFα, and GFP was assessed by flow cytometry. FIG. 22B shows Jurkat LTR-gfp cells were activated for 24 hrs with either 10 ng/ml TNFα or 1 μg/ml each of soluble α-CD3 and α-CD28 antibodies. GFP was then assessed by flow cytometry. FIG. 22C shows Jurkat LTR-gfp cells transduced with Lenti 1 vector encoding Vpx from SIV_{MAC}251, SIV_{RCM}02CM8081, or SIV_{MND2}5440, Vpr from SIV_{MND1}GB1, or SIV_{AGM}TAN1, or control no vpx Lenti 1, selected with puromycin, and activated for 24 hrs with 10 ng/ml TNFα. GFP expression was assessed by flow cytometry.

### DETAILED DESCRIPTION

Aspects of the disclosure are based on incorporation of viral sequences in constructs that are involved in integration of a nucleic acid insert (e.g., transgene) into a host cell chromosome. In some embodiments, minimization or elimination of these viral sequences permits larger nucleic acid inserts (e.g., transgenes encoding Cas nuclease) to be integrated into a host cell genome, as well as integration into cell types that are difficult to transfect and modify (e.g., dendritic cells).

### Constructs

Aspects of the disclosure relate to nucleic acid constructs encoding packagable vector RNAs that are capable of delivering large heterologous nucleic acid inserts (e.g., transgenes) to cells. As used herein, a “construct” is an artificially generated segment of nucleic acid that is transplanted into a target subject, tissue, or a cell.

Constructs of the present disclosure comprise nucleic acids encoding a promoter operably linked to a transgene. A “nucleic acid” may be a DNA sequence or an RNA sequence. In some embodiments, the nucleic acids of the present disclosure are isolated. As used herein, the term “isolated” means artificially produced. As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

A “promoter” refers to a DNA sequence recognized by the synthetic machinery of a cell, or introduced synthetic machinery, required to initiate the specific transcription of a gene. Promoters of the present disclosure are operably linked to transgenes. As used herein, “operably linked” sequences include both expression control sequences that are contiguous with the gene of interest and expression control sequences that act in trans or at a distance to control the gene of interest. Thus, a promoter region would be operably linked to a nucleic acid sequence if the promoter region were capable of effecting transcription of that DNA sequence such that the resulting transcript might be translated into the desired protein or polypeptide. Promoters that are native, constitutive, inducible, and/or tissue specific that are known in the art may be utilized. The phrases “operatively positioned,” “under control,” or “under transcriptional control” means that the promoter is in the correct location and orientation in relation to the nucleic acid transgene to control RNA polymerase initiation.

In some embodiments, constructs as described herein comprise more than one promoter (e.g., 2, 3, 4, 5, or more promoters). In some embodiments, one or more of the promoters in a construct described herein is an internal promoter. As used herein, an internal promoter refers to a promoter that is encoded in the transgene encoding the packagable vector RNA. In some embodiments, a construct comprises a first promoter and a second promoter (e.g., an internal second promoter), where the second promoter is operably linked to the heterologous nucleic acid. The second promoter may be any promoter described below.

Examples of constitutive promoters include, without limitation, the spleen focus forming viral promoter (SFFV), the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the β-actin promoter, the phosphoglycerol kinase (PGK) promoter, and the EFla promoter [Invitrogen]. In some embodiments, a promoter is a P2 promoter. In some embodiments, a promoter is a chicken β-actin (CBA) promoter. In some embodiments, a construct comprises two CBA promoters. In some embodiments, a construct comprises two CBA promoters separated by a CMV enhancer.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In some embodiments, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli.

In another embodiments, a tissue-specific promoter will be used to promote transgene expression in a particular tissue in a subject. Non-limiting examples of tissue-specific promoters include a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (α-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan.

Promoters of the present disclosure are operably linked to transgenes encoding packable vector RNA. A “transgene”, as used herein, refers to a gene that is artificially introduced into the genome of another organism. In the present disclosure, transgenes may comprise viral genes (e.g., retroviral genes). Transgenes of the present disclosure encode packagable vector RNA. As used herein, “packagable vector RNA” refers to RNA encoding any genetic element, such as a virus, virion, capsid, etc., that is capable of replication when associated with the proper control elements, and can be packaged into an appropriate capsules for delivery between and into cells.

Constructs of the present disclosure are utilized to infect host cells. In some embodiments, the packagable vector RNA of the present disclosure is packaged into capsids. A “capsid” as used herein, is the three-dimensional protein shell that encapsulates the genetic material (e.g., packagable vector RNA) of a virus. The capsid may also contain proteins that aid in the delivery of the packagable vector RNA to the surface of an into host cells.

In some embodiments, the packable vector RNA comprises 5′ and 3′ terminal repeats (TRs). “Terminal repeats” as used herein, are identical sequences of DNA or RNA that repeat hundreds or thousands of times. Terminal repeats of the present disclosure are utilized to mediate integration of viral nucleic acid (e.g., packable vector RNA) into another region of a host cell genome. Once integrated using the 5′ and 3′ TRs, the packagable vector RNA will be replicated by the host cell, thereby producing many packagable vector RNA molecules.

In some embodiments, the 5′ and 3′ TRs of the present disclosure are lentiviral long TRs. “Lentivirus” generally refers a family of retroviruses that cause chronic and severe infections in mammalian species. Lentiviruses infect and integrate their genomes into dividing and non-dividing cells (e.g., neurons). Nonlimiting examples of lentiviruses include human immunodeficiency virus, simian immunodeficiency virus (SIV), feline immunodeficiency virus (FIV), equine infectious anemia virus (EIAV), bovine immunodeficiency virus (BIV) and caprine arthritis encephalitis virus (CAEV). In some embodiments, lentiviral TRs are derived from HIV (e.g., share at least 50%, 60%, 70%, 80%, 90%, 95%, 99%, or 100% nucleic acid sequence identity with an HIV TR).

Lentiviral long terminal repeats (LLTRs) are RNA sequences that are partially transcribed in a host cell, followed by reverse transcription into complementary (cDNA) prior to integration of the virally-derived cDNA into the host cell genome. The 5′ and 3′ LLTRs regulate transcription of the packagable vector RNA in the host cell and mediate integration of the virally-derived cDNA into the host cell genome. The 5′ LLTR acts as a RNA polymerase II promoter upon integration into the host cell genome. In some embodiments, the 5′ LLTR is fused with the promoter operably linked to the transgene. The 3′ LLTR terminates transcription by adding a poly-A sequence at the 3′ end of the transcribed sequence.

The 5′ and 3′ LLTRs each contain multiple sequences, including unique 3 (U3), repeat (R), unique 5 (U5), and integrase substrate element. The U3 sequence is unique from the U5 sequence and is necessary for the activation of viral genomic RNA transcription. The R-element contains a region that binds to a trans-activator to activate reverse transcription. The U5 sequence is unique from the U3 sequence. The integrase substrate element is a sequence that is recognized and bound by the integrase protein. Integrase is a viral enzyme that catalyzes the integration of virally-derived DNA into the host cell genome.

In some embodiments, the 5′ and 3′ TRs of the present disclosure are truncated. “Truncated”, as used herein, refers to shortened nucleotide or amino acid sequences that retain the function of the full-length sequence. A truncated sequence may be at least 1, 2, 3, 4, 5, 6, 7, 8, 9, 10, 20, 30, 40, 50, 100, 200, or more nucleotides or amino acids shorter than the full length sequence from which it is derived. In some embodiments, the truncated sequences (e.g., truncated TR sequences) do not contain an R-element. In some embodiments, the truncated sequences (e.g., truncated TR sequences) do not contain an integrase substrate element. In some embodiments, the truncated sequences (e.g., truncated TR sequences) do not contain an R-element or an integrase substrate element. In some embodiments, the truncated sequences (e.g., truncated TR sequences) do not contain an R-element and an integrase substrate element. In some embodiments, the 5′ TR of a construct described herein is truncated. In some embodiments, the 3′ TR of a construct described herein is truncated. In some embodiments, the 5′ TR and the 3′ TR are truncated.

The 5′ and 3′ TRs of the present disclosure flank: a nucleocapsid protein packaging target site, a heterologous nucleic acid insert, and minimal intervening viral sequences. As used herein, the “nucleocapsid protein packaging target site” is a nucleic acid motif involved in regulating the packaging of a viral genome (e.g., packagable vector RNA) into a capsid. The nucleocapsid protein packaging target site, also referred to as packaging sequences, form secondary structures (e.g., stem-loop, bulges) that are recognized and bound by viral packaging proteins. Non-limiting examples of nucleocapsid protein packaging target sites include: psi (ψ) packaging element and infectious bronchitis virus packaging element.

A “heterologous nucleic acid insert”, as used herein, refers to a nucleic acid sequence to be inserted into a host cell genome that is not derived from the same species or cell type as the host cell. In some embodiments, the nucleic acid sequence is not derived from the same cell type as the host cell. In some embodiments, the nucleic acid sequence is not derived from the same species as the host cell. In some embodiments, the nucleic sequence is not derived from the cell type and the same species as the host cell.

The heterologous nucleic acid insert may encode a protein coding sequence or a non-protein coding sequence. Protein coding sequences are transcribed and translate into proteins or polypeptides. Non-protein coding sequences are transcribed and are not translated into proteins. Non-limiting examples of non-protein coding sequences include microRNAs (miRNAs), small interfering RNAs (siRNAs), artificial microRNAs (amiRNAs), long non-coding RNAs (lncRNAs), long intergenic non-coding RNAs (lincRNAs), ribosomal RNAs (rRNAs), transfer RNAs (tRNAs), enhancer RNAs, and super-enhancer RNAs. In some embodiments, non-protein coding sequences encode functional RNAs. As used herein, “functional RNAs” are RNAs that are not transcribed into proteins, but that fulfill a regulatory role in a cell. Non-limiting examples of functional RNAs include miRNAs, siRNAs, amiRNAs, lncRNAs, lincRNAs, rRNAs, and tRNAs.

Terminal repeats of the present disclosure flank minimal intervening viral sequences. As used herein, “minimal intervening viral sequences” are the shortest sequences derived from virus that allow the integration, replication, and packaging of the packagable vector RNA in a host cell. Non-limiting examples of virus from which the minimal intervening sequences may be derived include human immunodeficiency virus (HIV), infectious bronchitis virus (IBV), Moloney murine leukemia virus (MoMLV), and murine stem cell virus (MSCV).

In some embodiments, the minimal intervening viral sequences are in total up to 350 base pairs in length. In some embodiments, the minimal intervening viral sequences are in total between 200 and 400 base pairs in length. In some embodiments, the minimal intervening viral sequences are in total between 150 and 400 base pairs in length. In some embodiments, the minimal intervening viral sequences are in total between 100 and 350 base pairs in length. In some embodiments, the minimal intervening viral sequences are in total between 50 and 500 base pairs in length. In some embodiments, the packagable nucleic acid (e.g., packagable vector RNA) size is 1,900 bases, plus the size of the heterologous insert. In some embodiments, the packagable nucleic acid size is between 1,700 and 1,900 bases, plus the size of the heterologous insert. In some embodiments, the packagable nucleic acid size is between 1,000 and 2,000 bases, plus the size of the heterologous insert. In some embodiments, the packagable nucleic acid size is between 1,500 and 2,000 bases, plus the size of the heterologous insert.

In some embodiments, the TRs further flank a Rev protein response element (RRE). As used herein, a “Rev protein response element” is an RNA sequence bound by the Rev protein that allows the packagable vector RNA to be exported from the nucleus of the host after replication into the cytoplasm. The RRE forms multiple secondary structures (e.g., stems, loops, and bulges) that are recognized and bound by the Revl protein. In some embodiments, the sequence of the RRE and the Rev protein are derived from human immunodeficiency virus (HIV).

In some embodiments, the TRs further flank a polypurine tract. As used herein, a “polypurine tract” is a region containing numerous purine nucleotides (e.g., adenine, guanine), that is used as a primer for reverse transcription during viral replication. Reverse transcription of during viral replication is transcription of the viral RNA into DNA. In some embodiments, the polypurine tract contains only purines. In some embodiments, the polypurine tract contains the majority (e.g., over 50%) purines and some pyrimidines (e.g., cytosine, thymine, uracil). In some embodiments, the polypurine tract is located immediately adjacent to the 3′ LTR. In some embodiments, the polypurine tract is located near (e.g., within 50 bases, within 100 bases, within 200 bases, within 300 bases, etc.). the 3′ LTR.

In some embodiments, the TRs further flank a sequence encoding a group specific antigen (GAG) protein. The GAG proteins form the core of a viral capsid. The GAG protein contains numerous polypeptides, including matrix protein, capsid protein, space peptide 1, nucleocapsid protein, spacer peptide 2, and p6. The matrix (MA) protein comprises the N-terminus of GAG and is responsible for targeting GAG to the plasma membrane for release from an infected cell. The capsid protein (CA) is connected to the MA protein and forms the viral capsid. Spacer peptide 1 (SP1) is a short polypeptide connected to the CA protein that is cleaved upon production of the viral capsid. The nucleocapsid (NC) protein is connected to SP1 and forms the viral nucleocapsid. Spacer peptide 2 (SP2) is a short polypeptide that connects NC to the p6 polypeptide. The p6 polypeptide is at the C-terminus of the GAG polyprotein and recruits cellular proteins that promote virus capsid release from an infected cell.

In some embodiments, the present disclosure provides a construct comprising a sequence as set forth in SEQ ID NO: 10. In some embodiments, the present disclosure provides a construct comprising a sequence as set forth in SEQ ID NO: 11. In some embodiments, the construct is in a plasmid. In some embodiments, a construct comprising a sequence set forth in SEQ ID NO: 10 or 11 further comprises a heterologous nucleic acid insert, for example a heterologous nucleic acid insert that encodes one or more proteins or functional RNAs, such as a shRNA or miRNA, or a combination thereof.

### Nucleic Acids

In some aspects, the present disclosure provides isolated nucleic acids. The isolated nucleic acids comprise a heterologous nucleic acid insert flanked by TRs, wherein between the first TR and the second TR are present packaging sequences, nuclear export sequences, and minimal intervening viral sequences. The first TR and the second TR may be any TRs described herein. In some embodiments, the first TR is the 5′ TR and the second TR is the 3′ TR. In some embodiments, the first TR is the 3′ TR and the second TR is the 5′ TR.

In some aspects, the present disclosure provides transcribed nucleic acids. As used herein, “transcribed nucleic acids” refers to nucleic acids that have been transcribed in a cell (e.g., not produced recombinantly). In some embodiments, a transcribed nucleic acid is produced in a host cell. In some embodiments, a transcribed nucleic acid is produced not in a host cell.

In some aspects, transcribed nucleic acids comprise a heterologous nucleic acid insert flanked by TRs, wherein between the first TR and the heterologous nucleic acid insert, there are sequences that aid in the packaging and nuclear export of the transcribed nucleic acid and minimal intervening viral sequences.

In some embodiments, the heterologous nucleic acid insert is located between the nucleocapsid protein packaging site and the second TR. The heterologous nucleic acid insert may be operably linked to a promoter (e.g., internal promoter). In some embodiments, the internal promoter operably linked to the heterologous nucleic acid insert is spleen focus-forming virus (SFFV) promoter.

As used herein, “packaging sequences” are nucleic acid (e.g., RNA) sequences that promote packaging of a viral genome into a capsid. In some embodiments, packaging sequences of the nucleic acids comprise a psi (ψ) sequence and a polypurine tract sequence as described herein. In some embodiments, the ψ sequence precedes (is located 5′ to) the polypurine tract sequence. In some embodiments, the polypurine tract sequence precedes the ψ sequence.

As used herein a “nuclear export sequence” is a sequence that promotes the translocation of a replicated viral genome from the nucleus of a host cell to the cytoplasm for packaging. In some embodiments, a nuclear export sequence comprises the RRE. In some embodiments, the RRE is located between the ψ sequence and the polypurine tract sequence. In some embodiments, the RRE is located upstream of the ψ sequence and the polypurine tract sequence. In some embodiments, the RRE is located downstream of the ψ sequence and the polypurine tract sequence.

The nucleic acid comprises minimal intervening viral sequences. In some embodiments, the minimal intervening viral sequences are up to a total of 350 base pairs in length. In some embodiments, the minimal intervening viral sequences are a total of 25-350 base pairs, 50-300 base pairs, 100-350 base pairs, 125-200 base pairs, or 10-250 base pairs in length.

In some embodiments, nucleic acid packagable size is 1,900 bases, plus the size of the heterologous nucleic acid insert. As used herein, “nucleic acid packagable size” refers to the total length (in bases) of nucleic acids that will be packaged into a capsid protein. In some embodiments, the nucleic acid packagable size is 1,000-7,000, 1,900-8,000 bases, 3,000-6,000 bases, 2,000-5,000 bases, or 4,000-8,000 bases, plus the size of the nucleic acid insert.

### Heterologous Nucleic Acid Insert

The nucleic acids provided herein may contain a promoter that is located upstream of the 5′ TR. A promoter may be any promoter as described herein (e.g., constitutive, induced, native). In some embodiments, the promoter is a constitutive promoter. In some embodiments, the constitutive promoter is CMV. In some embodiments, the constitutive promoter is SV40. In some embodiments, the constitutive promoter is a fusion of CMV and SV40.

The nucleic acids described herein contain heterologous nucleic acid inserts. The heterologous nucleic acid inserts may be any that are described herein. In some embodiments, the heterologous nucleic acid insert encodes a functional RNA. In some embodiments, the functional RNA is a shRNA. In some embodiments, there is a selectable marker gene or a reporter gene upstream of the shRNA sequence. As used herein, a “selectable marker gene” encodes a protein that can be used to screen for cells by artificial selection. Non-limiting examples of selectable marker genes include antibiotic resistance genes (e.g., puromycin, ampicillin, kanamycin) and amino acid synthesis genes (e.g., URA3, TRYP, LEU). A “reporter gene” encodes a protein that can be used to screen for cells expressing or not expressing the reporter gene. Non-limiting examples of reporter genes include fluorescent genes (e.g., ZsGreen, green fluorescent protein, red fluorescent protein, yellow fluorescent protein, cyan fluorescent protein) and enzymatic genes (e.g., chloramphenicol acetyltransferase).

The disclosure relates, in part, to constructs having a heterologous nucleic acid insert configured to express one or more gene editing proteins to a cell. In some embodiments, the heterologous nucleic acid insert encodes a protein-coding gene. In some embodiments, the heterologous nucleic acid insert encodes a Cas nuclease. As used herein, “Cas nuclease” refers to clustered a regularly interspaced palindromic repeat (CRISPR)-associated nuclease. Cas nucleases cut nucleic acid (e.g., DNA, RNA) specific sequences, known as the protospacer adjacent motifs (PAMs), close to a target sequence in the nucleic acid. A Cas nuclease may any Cas nuclease known in the art (See, e.g., U.S. Pat. No. 8,697,359). In some embodiments, the Cas nuclease is Cas9 nuclease. In some embodiments, the Cas9 nuclease is from Streptococcus pyogenes, Neisseria meningitides, or Campylobacter jejuni.

In some embodiments, the heterologous nucleic acid insert encodes a microRNA. As used herein, a “microRNA” is a non-coding RNA molecule the decreases expression of a target gene or genes after base-pairing with and silencing mRNA molecules. mRNA molecules bound by microRNAs (miRNAs) are silenced by cleavage of the mRNA strand into two pieces, destabilization of the mRNA by shortening of its polyA tail, and/or less efficient translation of the mRNA into proteins. miRNAs can be processed into short-hairpin RNAs (shRNAs) in cells by the enzyme Dicer. shRNAs decreased gene expression of a target gene after binding mRNA molecules and stimulating the cleavage of the mRNA.

MicroRNAs of the disclosure may decrease gene expression of any gene that is transcribed into a mRNA molecule. In some embodiments, microRNAs decrease gene expression of genes that promote transcription. In some embodiments, miRNAs of the present disclosure target AGO1, AGO2, AGO3, DNMT3, HDAC1, HP1, SUV39H1, SUV39H2, PIWIL2, TRIM28, SETDB1, FAM208A, MPHOSPH8, PPHLN1, and/or MORC2. In some embodiments, miRNAs of the disclosure specifically bind to (e.g., hybridize or have a region of complementarity with) at least 3, 4, 5, 6, 7, 8, 9, 10, 11, 12, 13, 14, 15, 16, 17, 18, 19, 20, 21, 22, 23, 24, or 25 nucleotides of a gene encoding AGO1, AGO2, AGO3, DNMT3, HDAC1, HP1, SUV39H1, SUV39H2, PIWIL2, TRIM28, SETDB1, FAM208A, MPHOSPH8, PPHLN1, and/or MORC2.

### Host Cells

Methods of the present disclosure comprise delivering the constructs or nucleic acids described herein to host cells. As used herein, a “host cell” is a cell the integrates a heterologous nucleic acid insert of the present disclosure into its genome after being contacted with a construct, nucleic acid, or composition of the present disclosure. The host cell replicates the heterologous nucleic acid insert, which can be packaged into capsids that are released from the host cell. Non-limiting host cells of the present disclosure include human cells, mouse cells, rat cells, monkey cells, dog cells, or cat cells. In some embodiments, host cells are human cells. In some embodiments, a host cell is a primary human cell. As used herein, a “primary human cell” is a cell isolated directly from a tissue in a living human (e.g., biopsy) and established for growth in vitro. In some embodiments, the primary human cell is a dendritic cell.

In order to integrate and replicate heterologous nucleic acids inserts of the present disclosure, host cells must contain RNA polymerase. The RNA polymerase binds the 5′ TR and catalyzes transcription of the heterologous nucleic acid insert. In some embodiments, the RNA polymerase is endogenously expressed. In some embodiments, the RNA polymerase is exogenously expressed. As used herein, “endogenously expressed” refers to an RNA polymerase that is part of the genome of the host cell. As used herein, “exogenously expressed” refers to an RNA polymerase that is not part of the genome of the host cell. In some embodiments, the RNA polymerase is RNA polymerase II.

In order to package and release the heterologous nucleic acid insert, host cells of the present disclosure must express nucleic acid sequences that facilitate encapsulating and enveloping of the heterologous nucleic acid. To ensure that viruses do not reproduce spontaneously in host cells, the nucleic acid sequences that facilitate encapsulating and enveloping of the heterologous nucleic acid are contained in plasmids. As used herein, a “plasmid” is a small DNA molecule in a host cell that is physically separated from and replicates independent on the host cell genome. In some embodiments, the encapsulating and enveloping sequences are contained in (e.g., encoded by) the same plasmid. In some embodiments, the encapsulating and enveloping sequences are contained in (e.g., encoded by) separate plasmids.

Encapsulating nucleic acids encode genes for GAG, polymerase (pol), and Rev proteins. A GAG protein may be any GAG protein described herein. Pol protein contains both reverse transcriptase and integrase polypeptides. Reverse transcriptase is an enzyme that catalyzes the synthesis of complementary DNA (cDNA) from RNA (e.g., packagable vector RNA). Rev protein binds the RRE, as described previously. In some embodiments, the encapsulating sequences encode GAG, pol, and Rev proteins. In some embodiments, the encapsulating sequences encode GAG protein. In some embodiments, the encapsulating sequences encode pol proteins. In some embodiments, the encapsulating sequence encodes Rev proteins.

In some embodiments, the GAG, pol, and Rev encapsulating sequences are in (e.g., encoded by) the same plasmid. In some embodiments, the GAG, pol, and Rev encapsulating sequences are in (e.g., encoded by) 3 separate plasmids. In some embodiments, the GAG, pol, and Rev encapsulating sequences are in (e.g., encoded by) 2 separate plasmids.

Enveloping refers to the encapsulation of a capsid (e.g., viral capsid). Viral envelopes are derived from the host cell plasma membrane, and also contain viral glycoproteins. These viral glycoproteins bind receptor proteins on host cell membranes and help virus capsids to avoid the host immune system. In some embodiments, the viral envelope sequence encodes vesicular stomatitis virus G glycoprotein (VSVG). In some embodiments, the enveloping sequence is in the same plasmid of as the packaging sequences. In some embodiments, the enveloping sequence is in a separate plasmid from the packaging sequences.

In some embodiments, host cells of the present disclosure comprise viral particles. As used herein, “viral particles”, also known as “virions”, are viral nucleic acid (e.g., RNA) surrounded by a capsid protein. In some embodiments, the viral nucleic acid is transcribed nucleic acid, as described herein. In some embodiments, the viral nucleic acid is isolated nucleic acid, as described herein.

### Methods of Use

In some aspects, the present disclosure provides methods for efficient gene knockdown comprising infecting target cells with viral particles. Target cells may be any cells in a mammalian subject. Non-limiting examples of target cells include human cells, non-human primate cells, mouse cells, rat cells, dog cells, cat cells, cow cells, pig cells, or chicken cells. In some embodiments, the target cells are human cells. In some embodiments, human cells are primary human cells. Non-limiting examples of human primary cells include dendritic cells, neurons, natural killer cells, T cells, B cells, myocytes, osteoclasts, osteoblasts, chondrocytes, chondroclasts, glial cells, hepatocytes, renal cells, and epithelial cells. In some embodiments, the primary human cells are dendritic cells.

The viral particles may be any viral particles as described herein (e.g., transcribed nucleic acids, isolated nucleic acids). “Efficient gene knockdown”, as used herein, refers to a 40% decrease, a 45% decrease, a 50% decrease, a 55% decrease, a 60% decrease, a 65% decrease, a 70% decrease, a 75% decrease, an 80% decrease, an 85% decrease, a 90% decrease, a 95% decrease, or a 95% decrease in expression of the target gene (e.g., relative to expression of the target gene in a cell or subject prior to administration of a construct described herein). Non-limiting examples of target genes include AGO1, AGO2, AGO3, DNMT3, HDAC1, HP1, SUV39H1, SUV39H2, PIWIL2, TRIM28, SETDB1, FAM208A, MPHOSPH8, PPHLN1, and MORC2.

In some aspects, the present disclosure provides methods of delivering plasmids to a cell. The plasmids may contain any constructs or nucleic acids described herein. Non-limiting methods of delivering plasmids to a cell include: viral delivery (e.g., retroviral, lentiviral, etc.), transfection, electroporation, heat shock, liposomes, nanoparticles, microinjection, sonoporation, photoporation, magetofection, and hydroporation.

In some aspects, the present disclosure provides methods of infecting a host cell with viral particles. The viral particles may encapsulate any nucleic acids (e.g., isolated, transcribed) as described herein. Viral particles may be RNA-based viral particles (e.g., lentiviral, oncoretroviral, human foamy virus). Viral particles may be DNA-based viral particles (e.g., adenovirus, adeno-associated virus, herpes simplex virus).

In some embodiments, the host cell is in a subject that is infected with the viral particles. A subject is any mammal, including, but not limited to, a human, a non-human primate, a mouse, a rat, a dog, a cat, a cow, a pig, or a chicken. Viral particles may be administered to a subject by any method known in the art. Non-limiting methods of administering viral particles include intramuscular injection, intravenous injection, intra-arterial injection, inhalation, and ingestion.

In some aspects, the present disclosure provides compositions comprising a plurality of nucleic acids. As used herein, a “plurality” may be 2 or more, 10 or more, hundreds or more, thousands or more, millions or more, billions or more, or trillions or more nucleic acids. In some embodiments, the nucleic acids in the compositions are the same nucleic acids. In some embodiments, the nucleic acids in the compositions are different nucleic acids.

In some embodiments, compositions comprise a pharmaceutically acceptable carrier. As used herein, “carrier” includes any and all solvents, dispersion media, vehicles, coatings, diluents, antibacterial agents, antifungal agents, isotonic and absorption delaying agents, buffers, carrier solutions, suspensions, colloids, and the like. The use of such media and agents for pharmaceutically active substances is well known in the art. Supplementary active ingredients can also be incorporated into the compositions. The phrase “pharmaceutically acceptable” refers to molecular entities and compositions that do not produce an allergic or similar untoward reaction when administered to a host.

### Kits and Related Compositions

The agents described herein may, in some embodiments, be assembled into pharmaceutical or diagnostic or research kits to facilitate their use in therapeutic, diagnostic or research applications. A kit may include one or more containers housing the components of the disclosure and instructions for use. Specifically, such kits may include one or more agents described herein, along with instructions describing the intended application and the proper use of these agents. In certain embodiments agents in a kit may be in a pharmaceutical formulation and dosage suitable for a particular application and for a method of administration of the agents. Kits for research purposes may contain the components in appropriate concentrations or quantities for running various experiments.

In some embodiments, the instant disclosure relates to a kit for producing a packagable vector RNA, the kit comprising a container housing a nucleic acid encoding a promoter operably linked to a transgene encoding the packagable vector RNA. The packagable vector RNA may be any packagable vector RNA described herein. In some embodiments, the kit also comprises additional plasmids that contain nucleic acids that facilitate encapsulating and enveloping of the packagable vector RNA. In some embodiments, the plasmids encoding nucleic acids that facilitate encapsulating and enveloping are in separate plasmids. In some embodiments, the plasmids encoding nucleic acids that facilitate encapsulating and enveloping are in the same plasmid.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container.

Exemplary embodiments of the invention will be described in more detail by the following examples. These embodiments are exemplary of the invention, which one skilled in the art will recognize is not limited to the exemplary embodiments.

### EXAMPLES

### Example 1: Vector Development

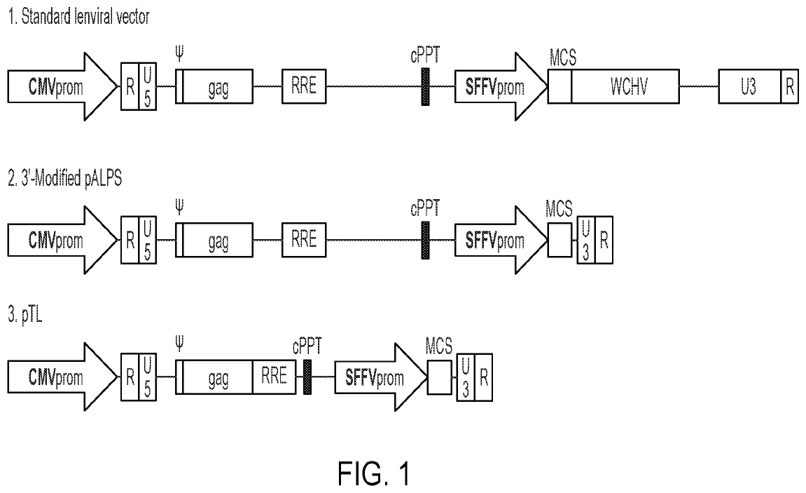

An overview schematic of lentiviral vector plasmids is shown in FIG. 1.

### Standard Vector to First Generation

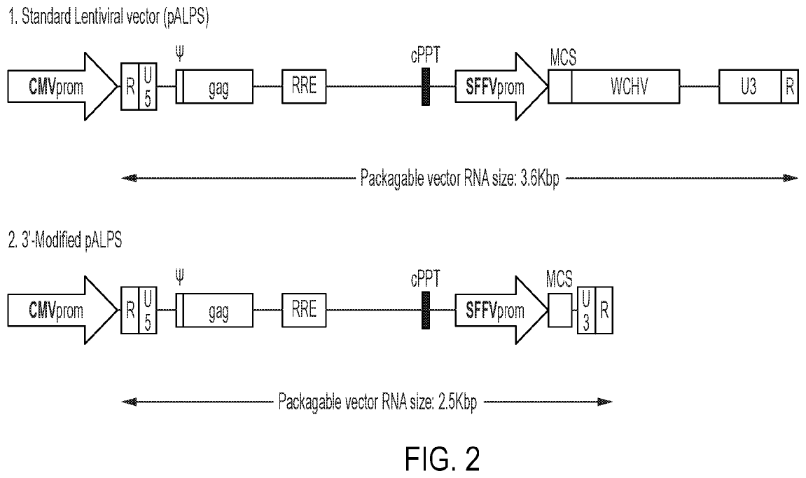

Certain components were deleted to make the lentiviral vector smaller. This increased vector titer in difficult to transduce cells, for transduction of large genes (e.g., CAS9), and for direct Illumina sequencing from the polyA (important for single cell RNA-Seq, for example). A schematic of the plasmids is shown in FIG. 2.

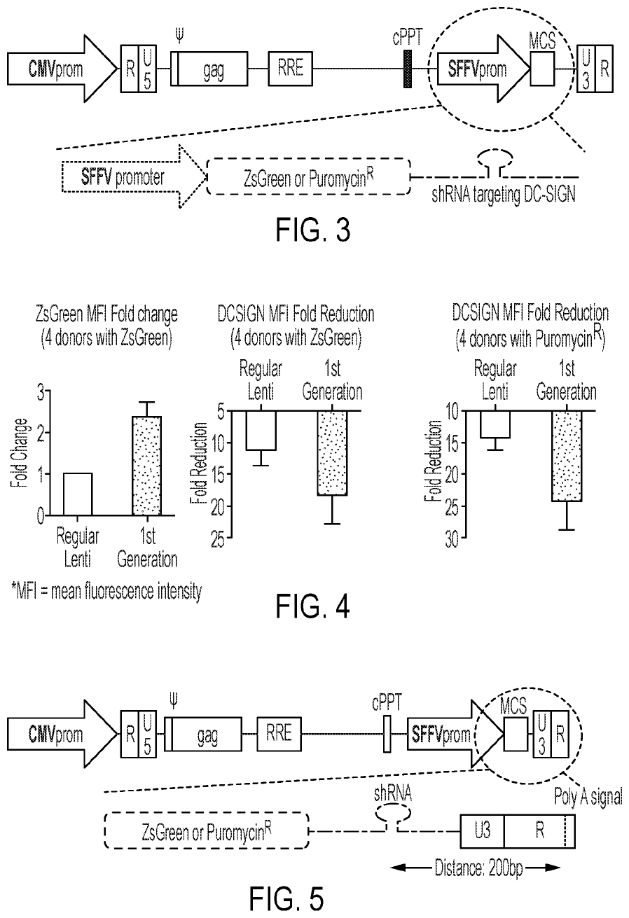

A first generation vector was tested using a knockdown construct on human primary dendritic cells. The vector expresses both ZsGreen (or Puromycin^{R}) and DC-SIGN knockdown shRNA. After 6 days transduction (with puromycin selection if using Puromycin^{R}), ZsGreen expression and DC-SIGN knockdown levels were checked by flow cytometry. A schematic of the text is shown in FIG. 3. Vector modification was found to enhance insert gene expression level. Higher protein expression levels and shRNA knockdown efficiency were achieved by modification (FIG. 4).

One application of the vector development is in single-cell sequencing with shRNA library screening. The single-cell RNA-Seq reads 100-300 base pair sequences from the 3′ end to the polyA site (FIG. 5). The distance in the developed vector allows that single-cell RNA-Seq reads the shRNA sequence directly, which does not need additional barcode sequences in shRNA library screening. This facilitates library cloning, leads to cost and labor savings, and prevents potential recombination between barcode and lentiviral RNA sequences.

Further Development from First Generation to Second Generation Vector

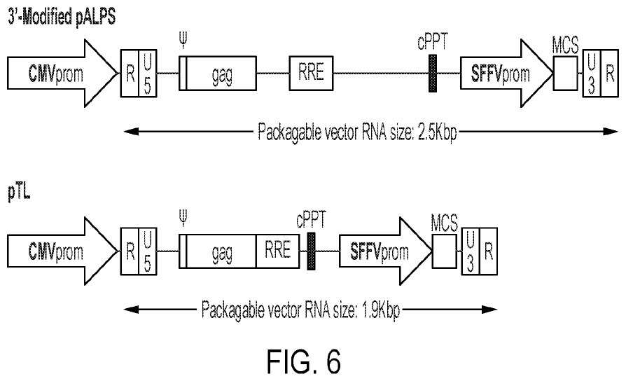

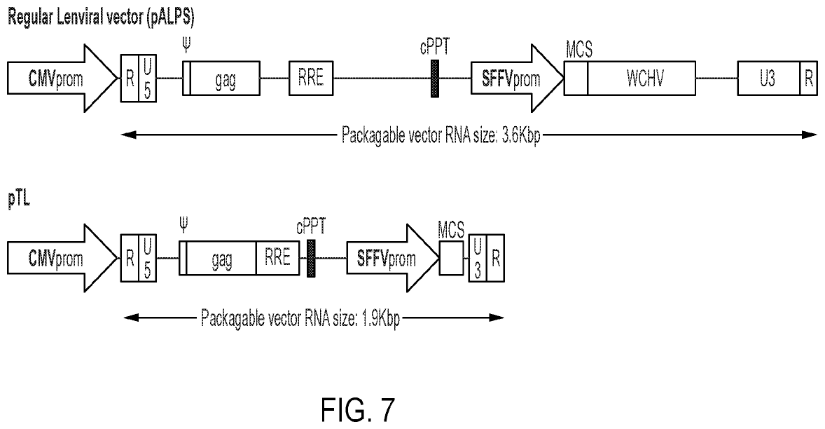

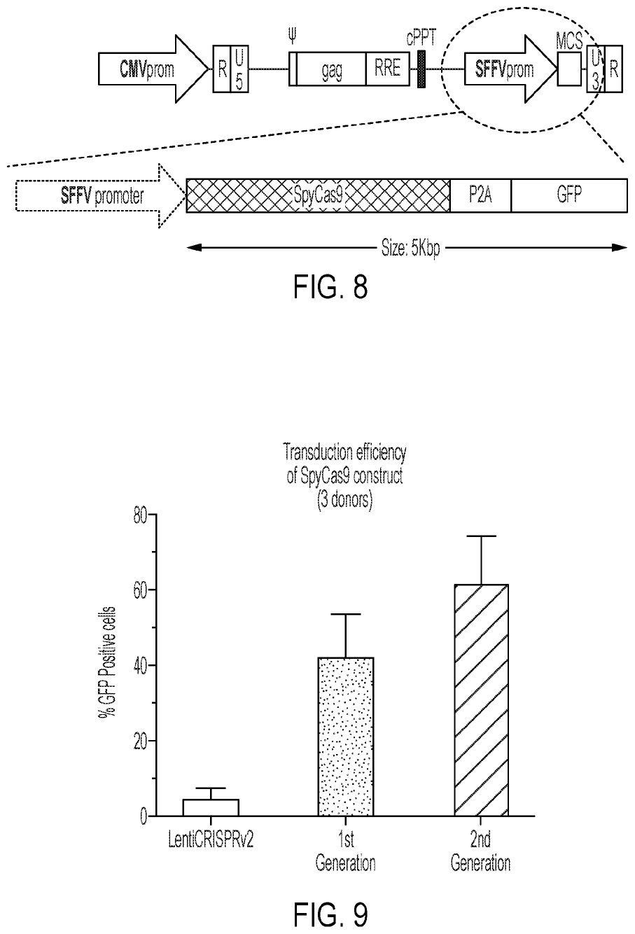

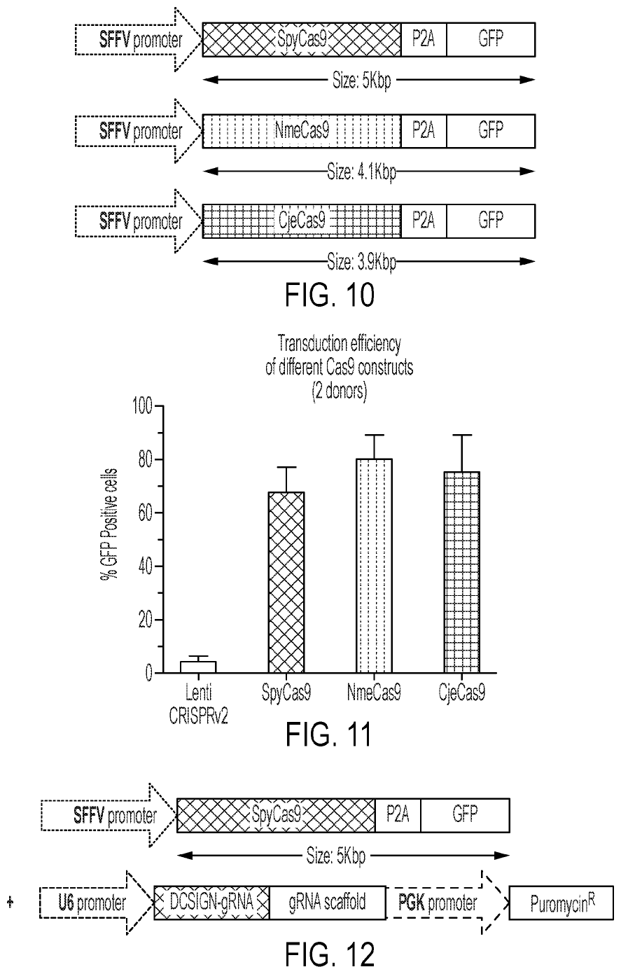

Overview schematics of first generation versus second generation and regular versus second generation lentiviral vectors are shown in FIGS. 6 and 7, respectively. First and second generation vectors for Cas9 transduction were tested on human primary dendritic cells (FIG. 8). The vector expresses both SpyCas9 (Cas9 from Streptococcus pyogenes) and GFP. After 6 days transduction, the percentage of GFP positive cells was checked by flow cytometry. LentiCRISPRv2 (Addgene #52961; replaced puromycin^{R} with GFP) was used as a control. The vectors were found to have increased transduction efficiency with Cas9 as test cargo (FIG. 9). Second generation vectors were tested for transduction of different Cas9 types on human primary dendritic cells (FIG. 10). The developed vector exhibited good transduction efficiencies on all Cas9 constructs (FIG. 11). The smaller insert construct showed higher transduction efficiency.

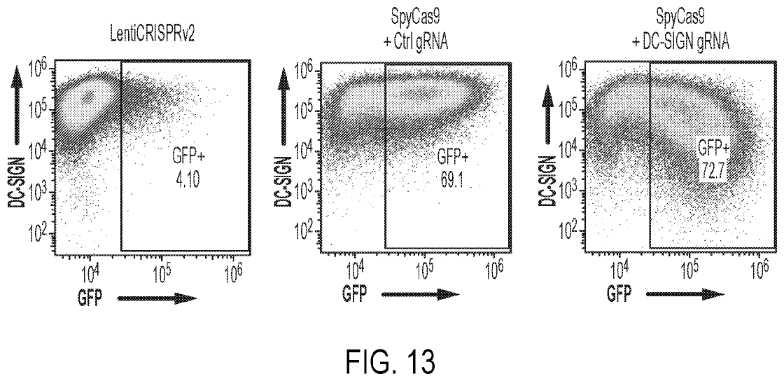

It was tested whether transduced Cas9s disrupt target gene expression in human primary dendritic cells (FIG. 12). The SpyCas9 construct and single gRNA construct targeting gene encoding the cell surface marker DC-SIGN were co-transduced. After 6 days transduction with puromycin selection, the percentage of GFP positive cells and DC-SIGN expression level were checked by flow cytometry. LentiCRISPRv2 (Addgene #52961; replaced puromycin^{R} with GFP) was used as a control. Transduced SpyCas9 disrupts cell surface levels of the protein encoded by the target gene, DC-SIGN (FIG. 13).

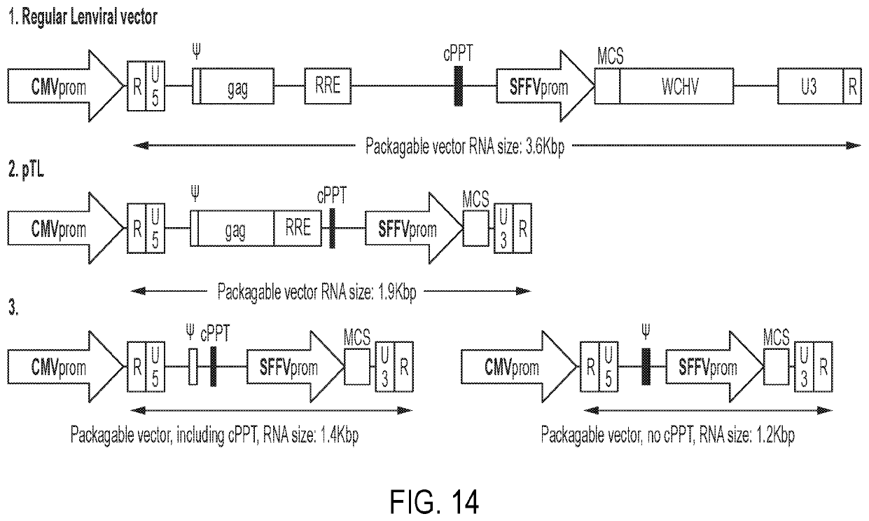

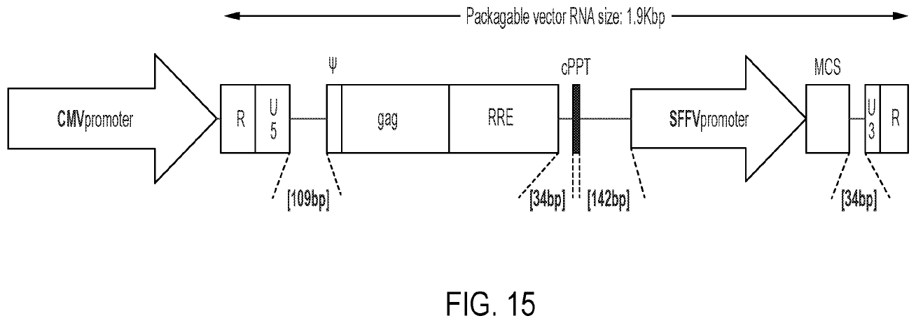

A third generation lentiviral vector is depicted in FIG. 14.

| Plasmid sequences | |
|---|---|
| pALPS | |
| (SEQ ID NO: 1) | |
| 1 | gtcgacggat cgggagatct cccgatcccc tatggtgcac tctcagtaca atctgctctg |
| 61 | atgccgcata gttaagccag tatctgctcc ctgcttgtgt gttggaggtc gctgagtagt |
| 121 | gcgcgagcaa aatttaagct acaacaaggc aaggcttgac cgacaattgc atgaagaatc |
| 181 | tgcttagggt taggcgtttt gcgctgcttc gcgatgtacg ggccagatat acgcgctgtg |
| 241 | gaatgtgtgt cagttagggt gtggaaagtc cccaggctcc ccagcaggca gaagtatgca |
| 301 | aagcatgcat ctcaattagt cagcaaccag gtgtggaaag tccccaggct ccccagcagg |
| 361 | cagaagtatg caaagcatgc atctcaatta gtcagcaacc atagtcccgc ccctaactcc |
| 421 | gcccatcccg cccctaactc cgcccagttc cgcccattct ccgccccatg gctgactaat |
| 481 | tttttttatt tatgcagagg ccgaggccgc ctctgcctct gagctattcc agaagtagtg |
| 541 | aggaggcttt tttggaggcc taggcttttg caaaaagctt tgacattgat tattgactag |
| 601 | ttattaatag taatcaatta cggggtcatt agttcatagc ccatatatgg agttccgcgt |
| 661 | tacataactt acggtaaatg gcccgcctgg ctgaccgccc aacgaccccc gcccattgac |
| 721 | gtcaataatg acgtatgttc ccatagtaac gccaataggg actttccatt gacgtcaatg |
| 781 | ggtggagtat ttacggtaaa ctgcccactt ggcagtacat caagtgtatc atatgccaag |
| 841 | tacgccccct attgacgtca atgacggtaa atggcccgcc tggcattatg cccagtacat |
| 901 | gaccttatgg gactttccta cttggcagta catctacgta ttagtcatcg ctattaccat |
| 961 | ggtgatgcgg ttttggcagt acatcaatgg gcgtggatag cggtttgact cacggggatt |
| 1021 | tccaagtctc caccccattg acgtcaatgg gagtttgttt tggcaccaaa atcaacggga |
| 1081 | ctttccaaaa tgtcgtaaca actccgcccc attgacgcaa atgggcggta ggcgtgtacg |
| 1141 | gtgggaggtc tatataagca gcgcgttttg cctgtactgg gtctctctgg ttagaccaga |
| 1201 | tctgagcctg ggagctctct ggctaactag ggaacccact gcttaagcct caataaagct |
| 1261 | tgccttgagt gcttcaagta gtgtgtgccc gtctgttgtg tgactctggt aactagagat |
| 1321 | ccctcagacc cttttagtca gtgtggaaaa tctctagcag tggcgcccga acagggactt |
| 1381 | gaaagcgaaa gggaaaccag aggagctctc tcgacgcagg actcggcttg ctgaagcgcg |
| 1441 | cacggcaaga ggcgaggggc ggcgactggt gagtacgcca aaaattttga ctagcggagg |
| 1501 | ctagaaggag agagatgggt gcgagagcgt cagtattaag cgggggagaa ttagatcgcg |
| 1561 | atgggaaaaa attcggttaa ggccaggggg aaagaaaaaa tataaattaa aacatatagt |
| 1621 | atgggcaagc agggagctag aacgattcgc agttaatcct ggcctgttag aaacatcaga |
| 1681 | aggctgtaga caaatactgg gacagctaca accatccctt cagacaggat cagaagaact |
| 1741 | tagatcatta tataatacag tagcaaccct ctattgtgtg catcaaagga tagagataaa |
| 1801 | agacaccaag gaagctttag acaagataga ggaagagcaa aacaaaagta agaccaccgc |
| 1861 | acagcaagcg gccggccgct gatcttcaga cctggaggag gagatatgag ggacaattgg |
| 1921 | agaagtgaat tatataaata taaagtagta aaaattgaac cattaggagt agcacccacc |
| 1981 | aaggcaaaga gaagagtggt gcagagagaa aaaagagcag tgggaatagg agctttgttc |
| 2041 | cttgggttct tgggagcagc aggaagcact atgggcgcag cgtcaatgac gctgacggta |
| 2101 | caggccagac aattattgtc tggtatagtg cagcagcaga acaatttgct gagggctatt |
| 2161 | gaggcgcaac agcatctgtt gcaactcaca gtctggggca tcaagcagct ccaggcaaga |
| 2221 | atcctggctg tggaaagata cctaaaggat caacagctcc tggggatttg gggttgctct |
| 2281 | ggaaaactca tttgcaccac tgctgtgcct tggaatgcta gttggagtaa taaatctctg |
| 2341 | gaacagattt ggaatcacac gacctggatg gagtgggaca gagaaattaa caattacaca |
| 2401 | agcttaatac actccttaat tgaagaatcg caaaaccagc aagaaaagaa tgaacaagaa |
| 2461 | ttattggaat tagataaatg ggcaagtttg tggaattggt ttaacataac aaattggctg |
| 2521 | tggtatataa aattattcat aatgatagta ggaggcttgg taggtttaag aatagttttt |
| 2581 | gctgtacttt ctatagtgaa tagagttagg cagggatatt caccattatc gtttcagacc |
| 2641 | cacctcccaa ccccgagggg acccgacagg cccgaaggaa tagaagaaga aggtggagag |
| 2701 | agagacagag acagatccat tcgattagtg aacggatcgg cactgcgtgc gccaattctg |
| 2761 | cagacaaatg gcagtattca tccacaattt taaaagaaaa ggggggattg gggggtacag |
| 2821 | tgcaggggaa agaatagtag acataatagc aacagacata caaactaaag aattacaaaa |
| 2881 | acaaattaca aaaattcaaa attttcgggt ttattacagg gacagcagag atccagtttg |
| 2941 | gttaattaac tgcagccccg ataaaataaa agattttatt tagtctccag aaaaaggggg |
| 3001 | gaatgaaaga ccccacctgt aggtttggca agctagctgc agtaacgcca ttttgcaagg |
| 3061 | catggaaaaa taccaaacca agaatagaga agttcagatc aagggcgggt acatgaaaat |
| 3121 | agctaacgtt gggccaaaca ggatatctgc ggtgagcagt ttcggccccg gcccggggcc |
| 3181 | aagaacagat ggtcaccgca gtttcggccc cggcccgagg ccaagaacag atggtcccca |
| 3241 | gatatggccc aaccctcagc agtttcttaa gacccatcag atgtttccag gctcccccaa |
| 3301 | ggacctgaaa tgaccctgcg ccttatttga attaaccaat cagcctgctt ctcgcttctg |
| 3361 | ttcgcgcgct tctgcttccc gagctctata aaagagctca caacccctca ctcggcgcgc |
| 3421 | cagtcctccg acagactgag tcgcccgggg gtctagaagc gctggatccg tttaaacgcg |
| 3481 | gccgcccagc acagtggctc gagccgcggg ttaactggcc agaattcacg cgtatcgata |
| 3541 | ccggtggccc ctggggccgc gatcgctaat caacctctgg attacaaaat ttgtgaaaga |
| 3601 | ttgactggta ttcttaacta tgttgctcct tttacgctat gtggatacgc tgctttaatg |
| 3661 | cctttgtatc atgctattgc ttcccgtatg gctttcattt tctcctcctt gtataaatcc |
| 3721 | tggttgctgt ctctttatga ggagttgtgg cccgttgtca ggcaacgtgg cgtggtgtgc |
| 3781 | actgtgtttg ctgacgcaac ccccactggt tggggcattg ccaccacctg tcagctcctt |
| 3841 | tccgggactt tcgctttccc cctccctatt gccacggcgg aactcatcgc cgcctgcctt |
| 3901 | gcccgctgct ggacaggggc tcggctgttg ggcactgaca attccgtggt gttgtcgggg |
| 3961 | aagctgacgt cctttccatg gctgctcgcc tgtgttgcca cctggattct gcgcgggacg |
| 4021 | tccttctgct acgtcccttc ggccctcaat ccagcggacc ttccttcccg cggcctgctg |
| 4081 | ccggctctgc ggcctcttcc gcgtcttcgc cttcgccctc agacgagtcg gatctccctt |
| 4141 | tgggccgcct ccccgcttaa tcgcgtcgag acctagaaaa acatggagca atcacaagta |
| 4201 | gcaatacagc agctaccaat gctgattgtg cctggctaga agcacaagag gaggaggagg |
| 4261 | tgggttttcc agtcacacct caggtacctt taagaccaat gacttacaag gcagctgtag |
| 4321 | atcttagcca ctttttaaaa gaaaaggggg gactggaagg gctaattcac tcccaacgaa |
| 4381 | gacaagatat ccttgatctg tggatctacc acacacaagg ctacttccct gattggcaga |
| 4441 | actacacacc agggccaggg atcagatatc cactgacctt tggatggtgc tacaagctag |
| 4501 | taccagttga gcaagagaag gtagaagaag ccaatgaagg agagaacacc cgcttgttac |
| 4561 | accctgtgag cctgcatggg atggatgacc cggagagaga agtattagag tggaggtttg |
| 4621 | acagccgcct agcatttcat cacatggccc gagagctgca tccggactgt actgggtctc |
| 4681 | tctggttaga ccagatctga gcctgggagc tctctggcta actagggaac ccactgctta |
| 4741 | agcctcaata aagcttgcct tgagtgcttc aagtagtgtg tgcccgtctg ttgtgtgact |
| 4801 | ctggtaacta gagatccctc agaccctttt agtcagtgtg gaaaatctct agcagggccc |
| 4861 | gtttcatgtg agcaaaaggc cagcaaaagg ccaggaaccg taaaaaggcc gcgttgctgg |
| 4921 | cgtttttcca taggctccgc ccccctgacg agcatcacaa aaatcgacgc tcaagtcaga |
| 4981 | ggtggcgaaa cccgacagga ctataaagat accaggcgtt tccccctgga agctccctcg |
| 5041 | tgcgctctcc tgttccgacc ctgccgctta ccggatacct gtccgccttt ctcccttcgg |
| 5101 | gaagcgtggc gctttctcat agctcacgct gtaggtatct cagttcggtg taggtcgttc |
| 5161 | gctccaagct gggctgtgtg cacgaacccc ccgttcagcc cgaccgctgc gccttatccg |
| 5221 | gtaactatcg tcttgagtcc aacccggtaa gacacgactt atcgccactg gcagcagcca |
| 5281 | ctggtaacag gattagcaga gcgaggtatg taggcggtgc tacagagttc ttgaagtggt |
| 5341 | ggcctaacta cggctacact agaagaacag tatttggtat ctgcgctctg ctgaagccag |
| 5401 | ttaccttcgg aaaaagagtt ggtagctctt gatccggcaa acaaaccacc gctggtagcg |
| 5461 | gtggtttttt tgtttgcaag cagcagatta cgcgcagaaa aaaaggatct caagaagatc |
| 5521 | ctttgatctt ttctacgggg tctgacgctc agtggaacga aaactcacgt taagggattt |
| 5581 | tggtcatgag attatcaaaa aggatcttca cctagatcct tttaaattaa aaatgaagtt |
| 5641 | ttaaatcaat ctaaagtata tatgagtaaa cttggtctga cagttaccaa tgcttaatca |
| 5701 | gtgaggcacc tatctcagcg atctgtctat ttcgttcatc catagttgcc tgactccccg |
| 5761 | tcgtgtagat aactacgata cgggagggct taccatctgg ccccagtgct gcaatgatac |
| 5821 | cgcgagaccc acgctcaccg gctccagatt tatcagcaat aaaccagcca gccggaaggg |
| 5881 | ccgagcgcag aagtggtcct gcaactttat ccgcctccat ccagtctatt aattgttgcc |
| 5941 | gggaagctag agtaagtagt tcgccagtta atagtttgcg caacgttgtt gccattgcta |
| 6001 | caggcatcgt ggtgtcacgc tcgtcgtttg gtatggcttc attcagctcc ggttcccaac |
| 6061 | gatcaaggcg agttacatga tcccccatgt tgtgcaaaaa agcggttagc tccttcggtc |
| 6121 | ctccgatcgt tgtcagaagt aagttggccg cagtgttatc actcatggtt atggcagcac |
| 6181 | tgcataattc tcttactgtc atgccatccg taagatgctt ttctgtgact ggtgagtact |
| 6241 | caaccaagtc attctgagaa tagtgtatgc ggcgaccgag ttgctcttgc ccggcgtcaa |
| 6301 | tacgggataa taccgcgcca catagcagaa ctttaaaagt gctcatcatt ggaaaacgtt |
| 6361 | cttcggggcg aaaactctca aggatcttac cgctgttgag atccagttcg atgtaaccca |
| 6421 | ctcgtgcacc caactgatct tcagcatctt ttactttcac cagcgtttct gggtgagcaa |
| 6481 | aaacaggaag gcaaaatgcc gcaaaaaagg gaataagggc gacacggaaa tgttgaatac |
| 6541 | tcatactctt cctttttcaa tattattgaa gcatttatca gggttattgt ctcatgagcg |
| 6601 | gatacatatt tgaatgtatt tagaaaaata aacaaatagg ggttccgcgc acatttcccc |
| 6661 | gaaaagtgcc acctgac |
| 3′ Modified pALPS (1st Generation Vector) | |
| (SEQ ID NO: 2) | |
| 1 | gtcgacggat cgggagatct cccgatcccc tatggtgcac tctcagtaca atctgctctg |
| 61 | atgccgcata gttaagccag tatctgctcc ctgcttgtgt gttggaggtc gctgagtagt |
| 121 | gcgcgagcaa aatttaagct acaacaaggc aaggcttgac cgacaattgc atgaagaatc |
| 181 | tgcttagggt taggcgtttt gcgctgcttc gcgatgtacg ggccagatat acgcgctgtg |
| 241 | gaatgtgtgt cagttagggt gtggaaagtc cccaggctcc ccagcaggca gaagtatgca |
| 301 | aagcatgcat ctcaattagt cagcaaccag gtgtggaaag tccccaggct ccccagcagg |
| 361 | cagaagtatg caaagcatgc atctcaatta gtcagcaacc atagtcccgc ccctaactcc |
| 421 | gcccatcccg cccctaactc cgcccagttc cgcccattct ccgccccatg gctgactaat |
| 481 | tttttttatt tatgcagagg ccgaggccgc ctctgcctct gagctattcc agaagtagtg |
| 541 | aggaggcttt tttggaggcc taggcttttg caaaaagctt tgacattgat tattgactag |
| 601 | ttattaatag taatcaatta cggggtcatt agttcatagc ccatatatgg agttccgcgt |
| 661 | tacataactt acggtaaatg gcccgcctgg ctgaccgccc aacgaccccc gcccattgac |
| 721 | gtcaataatg acgtatgttc ccatagtaac gccaataggg actttccatt gacgtcaatg |
| 781 | ggtggagtat ttacggtaaa ctgcccactt ggcagtacat caagtgtatc atatgccaag |
| 841 | tacgccccct attgacgtca atgacggtaa atggcccgcc tggcattatg cccagtacat |
| 901 | gaccttatgg gactttccta cttggcagta catctacgta ttagtcatcg ctattaccat |
| 961 | ggtgatgcgg ttttggcagt acatcaatgg gcgtggatag cggtttgact cacggggatt |
| 1021 | tccaagtctc caccccattg acgtcaatgg gagtttgttt tggcaccaaa atcaacggga |
| 1081 | ctttccaaaa tgtcgtaaca actccgcccc attgacgcaa atgggcggta ggcgtgtacg |
| 1141 | gtgggaggtc tatataagca gcgcgttttg cctgtactgg gtctctctgg ttagaccaga |
| 1201 | tctgagcctg ggagctctct ggctaactag ggaacccact gcttaagcct caataaagct |
| 1261 | tgccttgagt gcttcaagta gtgtgtgccc gtctgttgtg tgactctggt aactagagat |
| 1321 | ccctcagacc cttttagtca gtgtggaaaa tctctagcag tggcgcccga acagggactt |
| 1381 | gaaagcgaaa gggaaaccag aggagctctc tcgacgcagg actcggcttg ctgaagcgcg |
| 1441 | cacggcaaga ggcgaggggc ggcgactggt gagtacgcca aaaattttga ctagcggagg |
| 1501 | ctagaaggag agagatgggt gcgagagcgt cagtattaag cgggggagaa ttagatcgcg |
| 1561 | atgggaaaaa attcggttaa ggccaggggg aaagaaaaaa tataaattaa aacatatagt |
| 1621 | atgggcaagc agggagctag aacgattcgc agttaatcct ggcctgttag aaacatcaga |
| 1681 | aggctgtaga caaatactgg gacagctaca accatccctt cagacaggat cagaagaact |
| 1741 | tagatcatta tataatacag tagcaaccct ctattgtgtg catcaaagga tagagataaa |
| 1801 | agacaccaag gaagctttag acaagataga ggaagagcaa aacaaaagta agaccaccgc |
| 1861 | acagcaagcg gccggccgct gatcttcaga cctggaggag gagatatgag ggacaattgg |
| 1921 | agaagtgaat tatataaata taaagtagta aaaattgaac cattaggagt agcacccacc |
| 1981 | aaggcaaaga gaagagtggt gcagagagaa aaaagagcag tgggaatagg agctttgttc |
| 2041 | cttgggttct tgggagcagc aggaagcact atgggcgcag cgtcaatgac gctgacggta |
| 2101 | caggccagac aattattgtc tggtatagtg cagcagcaga acaatttgct gagggctatt |
| 2161 | gaggcgcaac agcatctgtt gcaactcaca gtctggggca tcaagcagct ccaggcaaga |
| 2221 | atcctggctg tggaaagata cctaaaggat caacagctcc tggggatttg gggttgctct |
| 2281 | ggaaaactca tttgcaccac tgctgtgcct tggaatgcta gttggagtaa taaatctctg |
| 2341 | gaacagattt ggaatcacac gacctggatg gagtgggaca gagaaattaa caattacaca |
| 2401 | agcttaatac actccttaat tgaagaatcg caaaaccagc aagaaaagaa tgaacaagaa |
| 2461 | ttattggaat tagataaatg ggcaagtttg tggaattggt ttaacataac aaattggctg |
| 2521 | tggtatataa aattattcat aatgatagta ggaggcttgg taggtttaag aatagttttt |
| 2581 | gctgtacttt ctatagtgaa tagagttagg cagggatatt caccattatc gtttcagacc |
| 2641 | cacctcccaa ccccgagggg acccgacagg cccgaaggaa tagaagaaga aggtggagag |
| 2701 | agagacagag acagatccat tcgattagtg aacggatcgg cactgcgtgc gccaattctg |
| 2761 | cagacaaatg gcagtattca tccacaattt taaaagaaaa ggggggattg gggggtacag |
| 2821 | tgcaggggaa agaatagtag acataatagc aacagacata caaactaaag aattacaaaa |
| 2881 | acaaattaca aaaattcaaa attttcgggt ttattacagg gacagcagag atccagtttg |
| 2941 | gttaattaac tgcagccccg ataaaataaa agattttatt tagtctccag aaaaaggggg |
| 3001 | gaatgaaaga ccccacctgt aggtttggca agctagctgc agtaacgcca ttttgcaagg |
| 3061 | catggaaaaa taccaaacca agaatagaga agttcagatc aagggcgggt acatgaaaat |
| 3121 | agctaacgtt gggccaaaca ggatatctgc ggtgagcagt ttcggccccg gcccggggcc |
| 3181 | aagaacagat ggtcaccgca gtttcggccc cggcccgagg ccaagaacag atggtcccca |
| 3241 | gatatggccc aaccctcagc agtttcttaa gacccatcag atgtttccag gctcccccaa |
| 3301 | ggacctgaaa tgaccctgcg ccttatttga attaaccaat cagcctgctt ctcgcttctg |
| 3361 | ttcgcgcgct tctgcttccc gagctctata aaagagctca caacccctca ctcggcgcgc |
| 3421 | cagtcctccg acagactgag tcgcccgggg gtctagaagc gctggatccg tttaaacgcg |
| 3481 | gccgcccagc acagtggctc gagccgcggg ttaactggcc agaattcacg cgtatcgata |
| 3541 | ccggtggccc ctggggccgc gatcgccagc tgtagatctt agccactttt taaaagaaaa |
| 3601 | ggggggactg gaagggctaa ctgcatccgg actgtactgg gtctctctgg ttagaccaga |
| 3661 | tctgagcctg ggagctctct ggctaactag ggaacccact gcttaagcct caataaagct |
| 3721 | tgccttgagt gcttcaagta gtgtgtgccc gtctgttgtg tgactctggt aactagagat |
| 3781 | ccctcagacc cttttagtca gtgtggaaaa tctctagcag ggcccgtttc atgtgagcaa |
| 3841 | aaggccagca aaaggccagg aaccgtaaaa aggccgcgtt gctggcgttt ttccataggc |
| 3901 | tccgcccccc tgacgagcat cacaaaaatc gacgctcaag tcagaggtgg cgaaacccga |
| 3961 | caggactata aagataccag gcgtttcccc ctggaagctc cctcgtgcgc tctcctgttc |
| 4021 | cgaccctgcc gcttaccgga tacctgtccg cctttctccc ttcgggaagc gtggcgcttt |
| 4081 | ctcatagctc acgctgtagg tatctcagtt cggtgtaggt cgttcgctcc aagctgggct |
| 4141 | gtgtgcacga accccccgtt cagcccgacc gctgcgcctt atccggtaac tatcgtcttg |
| 4201 | agtccaaccc ggtaagacac gacttatcgc cactggcagc agccactggt aacaggatta |
| 4261 | gcagagcgag gtatgtaggc ggtgctacag agttcttgaa gtggtggcct aactacggct |
| 4321 | acactagaag aacagtattt ggtatctgcg ctctgctgaa gccagttacc ttcggaaaaa |
| 4381 | gagttggtag ctcttgatcc ggcaaacaaa ccaccgctgg tagcggtggt ttttttgttt |
| 4441 | gcaagcagca gattacgcgc agaaaaaaag gatctcaaga agatcctttg atcttttcta |
| 4501 | cggggtctga cgctcagtgg aacgaaaact cacgttaagg gattttggtc atgagattat |
| 4561 | caaaaaggat cttcacctag atccttttaa attaaaaatg aagttttaaa tcaatctaaa |
| 4621 | gtatatatga gtaaacttgg tctgacagtt accaatgctt aatcagtgag gcacctatct |
| 4681 | cagcgatctg tctatttcgt tcatccatag ttgcctgact ccccgtcgtg tagataacta |
| 4741 | cgatacggga gggcttacca tctggcccca gtgctgcaat gataccgcga gacccacgct |
| 4801 | caccggctcc agatttatca gcaataaacc agccagccgg aagggccgag cgcagaagtg |
| 4861 | gtcctgcaac tttatccgcc tccatccagt ctattaattg ttgccgggaa gctagagtaa |
| 4921 | gtagttcgcc agttaatagt ttgcgcaacg ttgttgccat tgctacaggc atcgtggtgt |
| 4981 | cacgctcgtc gtttggtatg gcttcattca gctccggttc ccaacgatca aggcgagtta |
| 5041 | catgatcccc catgttgtgc aaaaaagcgg ttagctcctt cggtcctccg atcgttgtca |
| 5101 | gaagtaagtt ggccgcagtg ttatcactca tggttatggc agcactgcat aattctctta |
| 5161 | ctgtcatgcc atccgtaaga tgcttttctg tgactggtga gtactcaacc aagtcattct |
| 5221 | gagaatagtg tatgcggcga ccgagttgct cttgcccggc gtcaatacgg gataataccg |
| 5281 | cgccacatag cagaacttta aaagtgctca tcattggaaa acgttcttcg gggcgaaaac |
| 5341 | tctcaaggat cttaccgctg ttgagatcca gttcgatgta acccactcgt gcacccaact |
| 5401 | gatcttcagc atcttttact ttcaccagcg tttctgggtg agcaaaaaca ggaaggcaaa |
| 5461 | atgccgcaaa aaagggaata agggcgacac ggaaatgttg aatactcata ctcttccttt |
| 5521 | ttcaatatta ttgaagcatt tatcagggtt attgtctcat gagcggatac atatttgaat |
| 5581 | gtatttagaa aaataaacaa ataggggttc cgcgcacatt tccccgaaaa gtgccacctg |
| 5641 | ac |
| pTL (2nd Generation Vector) | |
| (SEQ ID NO: 3) | |
| 1 | gtcgacggat cgggagatct cccgatcccc tatggtgcac tctcagtaca atctgctctg |
| 61 | atgccgcata gttaagccag tatctgctcc ctgcttgtgt gttggaggtc gctgagtagt |
| 121 | gcgcgagcaa aatttaagct acaacaaggc aaggcttgac cgacaattgc atgaagaatc |
| 181 | tgcttagggt taggcgtttt gcgctgcttc gcgatgtacg ggccagatat acgcgctgtg |
| 241 | gaatgtgtgt cagttagggt gtggaaagtc cccaggctcc ccagcaggca gaagtatgca |
| 301 | aagcatgcat ctcaattagt cagcaaccag gtgtggaaag tccccaggct ccccagcagg |
| 361 | cagaagtatg caaagcatgc atctcaatta gtcagcaacc atagtcccgc ccctaactcc |
| 421 | gcccatcccg cccctaactc cgcccagttc cgcccattct ccgccccatg gctgactaat |
| 481 | tttttttatt tatgcagagg ccgaggccgc ctctgcctct gagctattcc agaagtagtg |
| 541 | aggaggcttt tttggaggcc taggcttttg caaaaagctt tgacattgat tattgactag |
| 601 | ttattaatag taatcaatta cggggtcatt agttcatagc ccatatatgg agttccgcgt |
| 661 | tacataactt acggtaaatg gcccgcctgg ctgaccgccc aacgaccccc gcccattgac |
| 721 | gtcaataatg acgtatgttc ccatagtaac gccaataggg actttccatt gacgtcaatg |
| 781 | ggtggagtat ttacggtaaa ctgcccactt ggcagtacat caagtgtatc atatgccaag |
| 841 | tacgccccct attgacgtca atgacggtaa atggcccgcc tggcattatg cccagtacat |
| 901 | gaccttatgg gactttccta cttggcagta catctacgta ttagtcatcg ctattaccat |
| 961 | ggtgatgcgg ttttggcagt acatcaatgg gcgtggatag cggtttgact cacggggatt |
| 1021 | tccaagtctc caccccattg acgtcaatgg gagtttgttt tggcaccaaa atcaacggga |
| 1081 | ctttccaaaa tgtcgtaaca actccgcccc attgacgcaa atgggcggta ggcgtgtacg |
| 1141 | gtgggaggtc tatataagca gcgcgttttg cctgtactgg gtctctctgg ttagaccaga |
| 1201 | tctgagcctg ggagctctct ggctaactag ggaacccact gcttaagcct caataaagct |
| 1261 | tgccttgagt gcttcaagta gtgtgtgccc gtctgttgtg tgactctggt aactagagat |
| 1321 | ccctcagacc cttttagtca gtgtggaaaa tctctagcag tggcgcccga acagggactt |
| 1381 | gaaagcgaaa gggaaaccag aggagctctc tcgacgcagg actcggcttg ctgaagcgcg |
| 1441 | cacggcaaga ggcgaggggc ggcgactgac gagtacgcca aaaattttga ctagcggagg |
| 1501 | ctagaaggag agagatgggt gcgagagcgt cagtattaag cgggggagaa ttagatcgcg |
| 1561 | atgggaaaaa attcggttaa ggccaggggg aaagaaaaaa tataaattaa aacatatagt |
| 1621 | atgggcaagc agggagctag aacgattcgc agttaatcct ggcctgttag aaacatcaga |
| 1681 | aggctgtaga caaatactgg gacagctaca accatccctt cagacaggat cagaagaact |
| 1741 | tagatcatta tataatacag tagcaaccct ctattgtgtg catcaaagga tagagataaa |
| 1801 | agacaccaag gaagctttag acaagataga ggaagagcaa aacaaaagta agaccaccgc |
| 1861 | acagcaagcg gccggccgct gaataggagc tttgttcctt gggttcttgg gagcagcagg |
| 1921 | aagcactatg ggcgcagcgt caatgacgct gacggtacag gccagacaat tattgtctgg |
| 1981 | tatagtgcag cagcagaaca atttgctgag ggctattgag gcgcaacagc atctgttgca |
| 2041 | actcacagtc tggggcatca agcagctcca ggcaagaatc ctggctgtgg aaagatacct |
| 2101 | aaaggatcaa cagctcctgg gggtatacac aaatggcagt attcatccac aattttaaaa |
| 2161 | gaaaaggggg gattgggggg tacagtgcag gggaaagaat agtagacata atagcaacag |
| 2221 | acatacaaac taaagaatta caaaaacaaa ttacaaaaat tcaaaatttt cgggtttatt |
| 2281 | acagggacag cagagatcca gtttggttaa ttaactgcag ccccgataaa ataaaagatt |
| 2341 | ttatttagtc tccagaaaaa ggggggaatg aaagacccca cctgtaggtt tggcaagcta |
| 2401 | gctgcagtaa cgccattttg caaggcatgg aaaaatacca aaccaagaat agagaagttc |
| 2461 | agatcaaggg cgggtacatg aaaatagcta acgttgggcc aaacaggata tctgcggtga |
| 2521 | gcagtttcgg ccccggcccg gggccaagaa cagatggtca ccgcagtttc ggccccggcc |
| 2581 | cgaggccaag aacagatggt ccccagatat ggcccaaccc tcagcagttt cttaagaccc |
| 2641 | atcagatgtt tccaggctcc cccaaggacc tgaaatgacc ctgcgcctta tttgaattaa |
| 2701 | ccaatcagcc tgcttctcgc ttctgttcgc gcgcttctgc ttcccgagct ctataaaaga |
| 2761 | gctcacaacc cctcactcgg cgcgccagtc ctccgacaga ctgagtcgcc cgggggtcta |
| 2821 | gaagcgctgg atccgtttaa acgcggccgc ccagcacagt ggctcgagcc gcgggttaac |
| 2881 | tggccagaat tcacgcgtat cgataccggt ggcccctggg gccgcgatcg ccagctgtag |
| 2941 | atcttagcca ctttttaaaa gaaaaggggg gactggaagg gctaactgca tccggactgt |
| 3001 | actgggtctc tctggttaga ccagatctga gcctgggagc tctctggcta actagggaac |
| 3061 | ccactgctta agcctcaata aagcttgcct tgagtgcttc aagtagtgtg tgcccgtctg |
| 3121 | ttgtgtgact ctggtaacta gagatccctc agaccctttt agtcagtgtg gaaaatctct |
| 3181 | agcagggccc gtttcatgtg agcaaaaggc cagcaaaagg ccaggaaccg taaaaaggcc |
| 3241 | gcgttgctgg cgtttttcca taggctccgc ccccctgacg agcatcacaa aaatcgacgc |
| 3301 | tcaagtcaga ggtggcgaaa cccgacagga ctataaagat accaggcgtt tccccctgga |
| 3361 | agctccctcg tgcgctctcc tgttccgacc ctgccgctta ccggatacct gtccgccttt |
| 3421 | ctcccttcgg gaagcgtggc gctttctcat agctcacgct gtaggtatct cagttcggtg |
| 3481 | taggtcgttc gctccaagct gggctgtgtg cacgaacccc ccgttcagcc cgaccgctgc |
| 3541 | gccttatccg gtaactatcg tcttgagtcc aacccggtaa gacacgactt atcgccactg |
| 3601 | gcagcagcca ctggtaacag gattagcaga gcgaggtatg taggcggtgc tacagagttc |
| 3661 | ttgaagtggt ggcctaacta cggctacact agaagaacag tatttggtat ctgcgctctg |
| 3721 | ctgaagccag ttaccttcgg aaaaagagtt ggtagctctt gatccggcaa acaaaccacc |
| 3781 | gctggtagcg gtggtttttt tgtttgcaag cagcagatta cgcgcagaaa aaaaggatct |
| 3841 | caagaagatc ctttgatctt ttctacgggg tctgacgctc agtggaacga aaactcacgt |
| 3901 | taagggattt tggtcatgag attatcaaaa aggatcttca cctagatcct tttaaattaa |
| 3961 | aaatgaagtt ttaaatcaat ctaaagtata tatgagtaaa cttggtctga cagttaccaa |
| 4021 | tgcttaatca gtgaggcacc tatctcagcg atctgtctat ttcgttcatc catagttgcc |
| 4081 | tgactccccg tcgtgtagat aactacgata cgggagggct taccatctgg ccccagtgct |
| 4141 | gcaatgatac cgcgagaccc acgctcaccg gctccagatt tatcagcaat aaaccagcca |
| 4201 | gccggaaggg ccgagcgcag aagtggtcct gcaactttat ccgcctccat ccagtctatt |
| 4261 | aattgttgcc gggaagctag agtaagtagt tcgccagtta atagtttgcg caacgttgtt |
| 4321 | gccattgcta caggcatcgt ggtgtcacgc tcgtcgtttg gtatggcttc attcagctcc |
| 4381 | ggttcccaac gatcaaggcg agttacatga tcccccatgt tgtgcaaaaa agcggttagc |
| 4441 | tccttcggtc ctccgatcgt tgtcagaagt aagttggccg cagtgttatc actcatggtt |
| 4501 | atggcagcac tgcataattc tcttactgtc atgccatccg taagatgctt ttctgtgact |
| 4561 | ggtgagtact caaccaagtc attctgagaa tagtgtatgc ggcgaccgag ttgctcttgc |
| 4621 | ccggcgtcaa tacgggataa taccgcgcca catagcagaa ctttaaaagt gctcatcatt |
| 4681 | ggaaaacgtt cttcggggcg aaaactctca aggatcttac cgctgttgag atccagttcg |
| 4741 | atgtaaccca ctcgtgcacc caactgatct tcagcatctt ttactttcac cagcgtttct |
| 4801 | gggtgagcaa aaacaggaag gcaaaatgcc gcaaaaaagg gaataagggc gacacggaaa |
| 4861 | tgttgaatac tcatactctt cctttttcaa tattattgaa gcatttatca gggttattgt |
| 4921 | ctcatgagcg gatacatatt tgaatgtatt tagaaaaata aacaaatagg ggttccgcgc |
| 4981 | acatttcccc gaaaagtgcc acctgac |

### Example 2: Primate Immunodeficiency Virus Vpx and Vpr Counteract Transcriptional Repression of Proviruses by the HUSH Complex

Drugs that inhibit HIV-1 replication and prevent progression to AIDS do not eliminate HIV-1 proviruses from the chromosomes of long-lived CD4⁺ memory T cells. To escape eradication by these antiviral drugs, or by the host immune system, HIV-1 exploits poorly defined host factors that silence provirus transcription. These same factors, though, must be overcome by all retroviruses, including HIV-1 and other primate immunodeficiency viruses, in order to activate provirus transcription and produce new virus. Here it is shown that Vpx and Vpr, proteins from a wide range of primate immunodeficiency viruses, activate provirus transcription in human CD4⁺ T cells. Provirus activation required the DCAF1 adaptor that links Vpx and Vpr to the CUL4A/B ubiquitin ligase complex, but did not require degradation of SAMHD1, a well-characterized target of Vpx and Vpr. A loss-of-function screen for transcription silencing factors that mimic the effect of Vpx on provirus silencing identified all components of the Human Silencing Hub (HUSH) complex, FAM208A (TASOR/RAP140), MPHOSPH8 (MPP8), PPHLN1 (PERIPHILIN), and MORC2. Vpx associated with the HUSH complex components and decreased steady-state levels of these proteins in a DCAF-dependent manner. Finally, vpx and FAM208A knockdown accelerated HIV-1 and SIV_{MAC} replication kinetics in CD4⁺ T cells to a similar extent, though HIV-2 replication required either vpx or FAM208A disruption. These results demonstrate that the HUSH complex restricts HIV-1 transcription and thereby contributes to provirus latency. To counteract this restriction and activate provirus expression, primate immunodeficiency viruses encode Vpx and Vpr proteins that degrade HUSH complex components.

When provided in trans, many primate immunodeficiency virus Vpx and Vpr orthologues increase HIV-1 reverse transcription and transduction efficiency in dendritic cells, macrophages, and resting CD4⁺ T cells. As substrate adaptor proteins for the DCAF1-CUL4A/B E3 ubiquitin ligase, Vpx and Vpr increase the concentration of deoxynucleotide triphosphate (dNTP) levels in target cells by degrading the deoxynucleotidetriphosphate (dNTP) hydrolase SAMHD1. Nonetheless, Vpx and Vpr have additional effects on expression of transduced reporter genes that are not explained by SAMHD1 degradation or by increase in dNTP concentration.

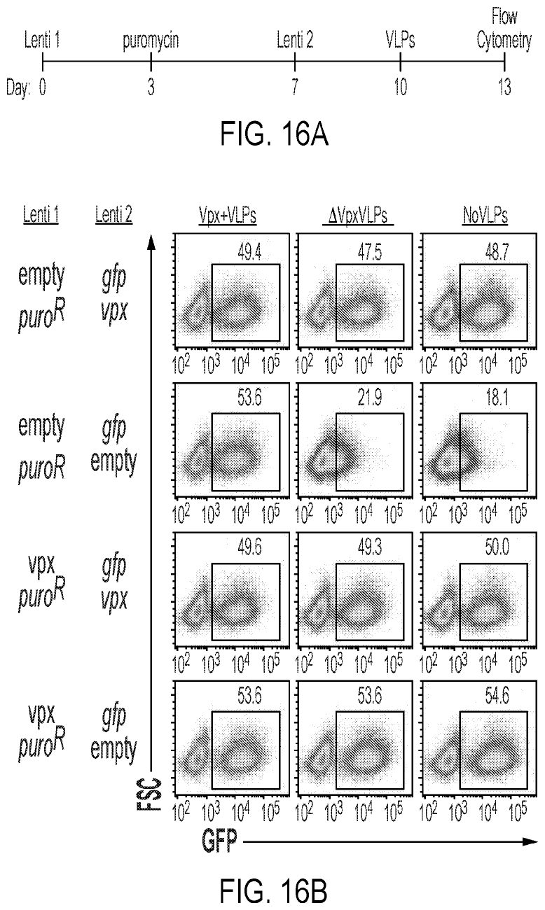

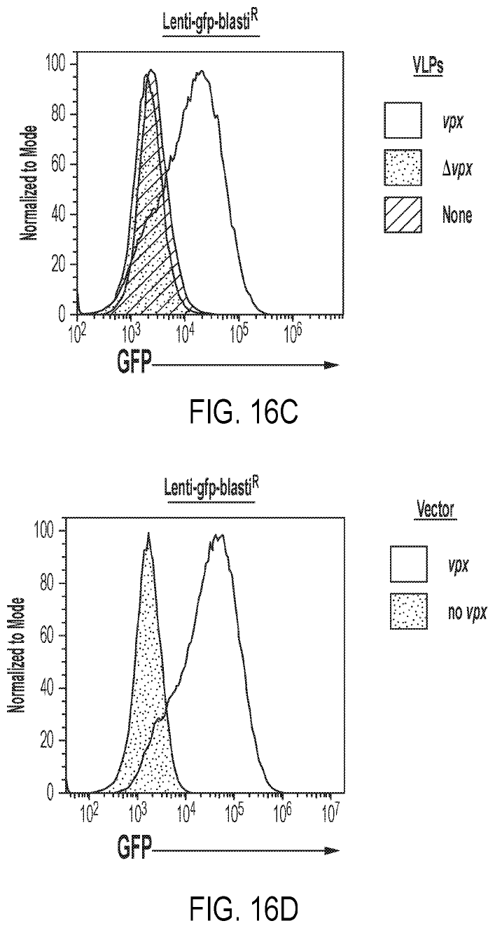

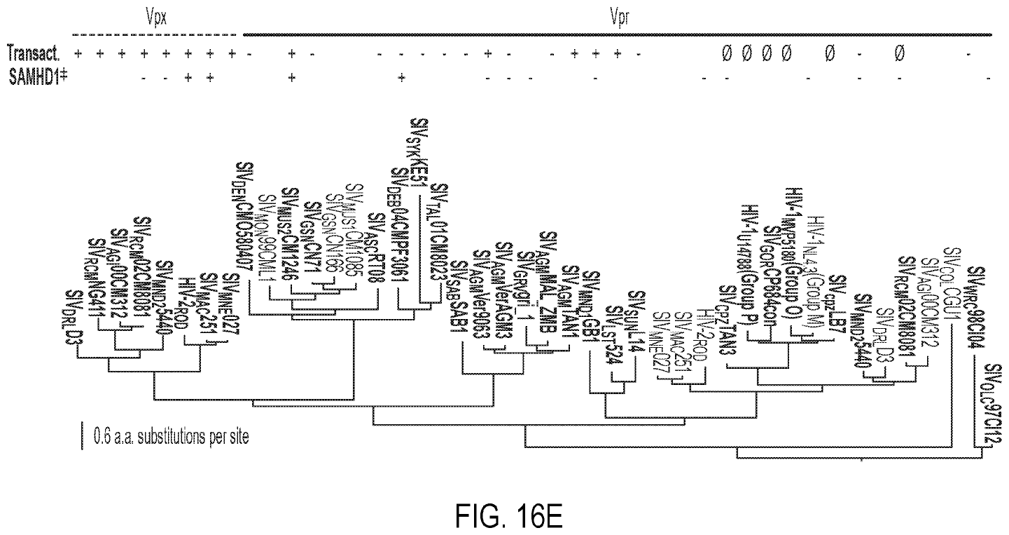

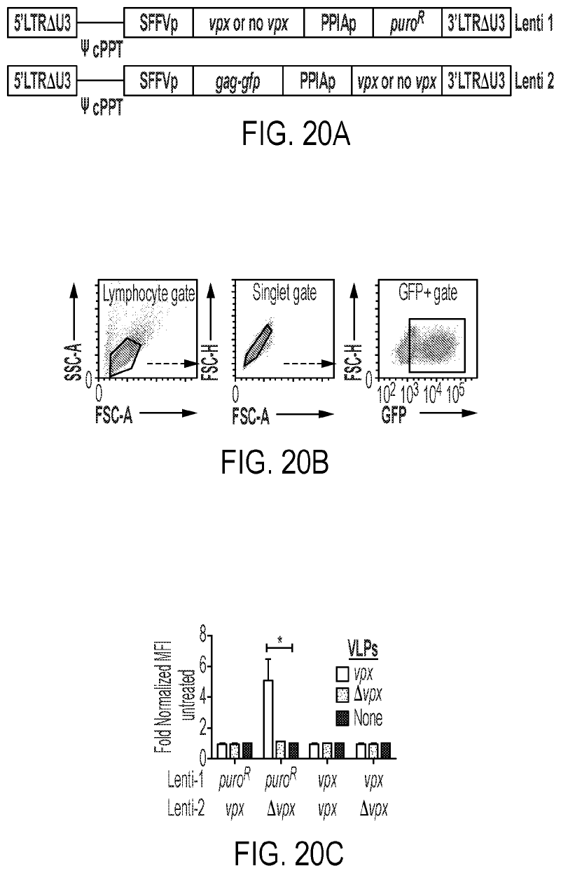

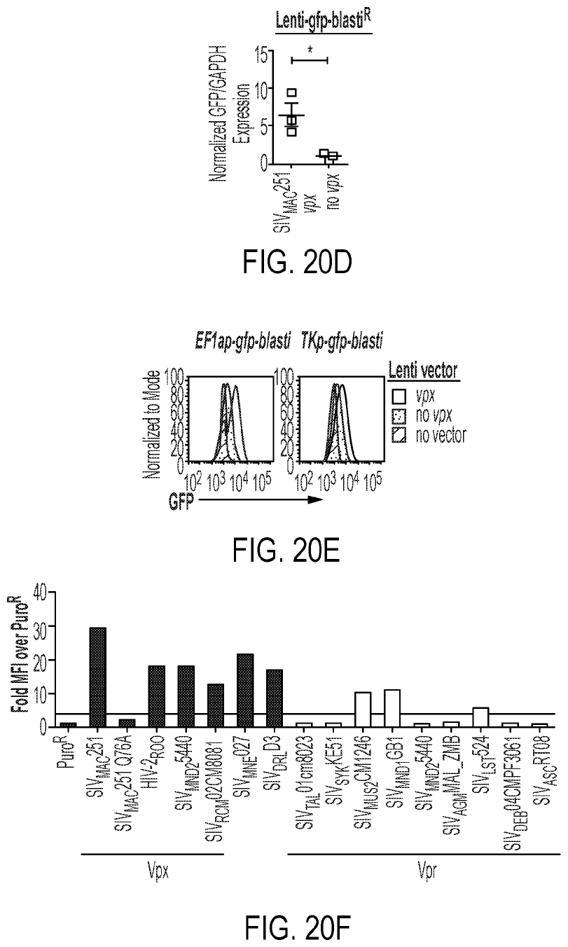

To better understand the effect on provirus reporter gene expression, vpx was introduced before, during, or after transduction of a reporter gene (FIG. 16A). Jurkat CD4⁺ T cells were transduced with a dual-promoter, lentiviral vector that expresses codon-optimized SIV_{MAC}251 vpx from the spleen focus forming virus (SFFV) promoter and puromycin acetyltransferase (puro^{R}) from the PPIA (CypA) promoter (Lenti 1 in the FIG. 16A time-line, FIG. 20A and Table 1). A control Lenti 1 vector was used that lacks vpx (FIG. 20A). Puromycin was added to the culture on day three to select those cells that had been transduced with Lenti 1. On day seven, cells were transduced with a second lentivector bearing a codon-optimized gag-gfp reporter gene expressed from the SFFV promoter, as well as SIV_{MAC}251 vpx expressed from the CypA promoter (Lenti 2 in the FIG. 16A timeline and FIG. 20A). A control Lenti 2 vector was used that lacks vpx (FIG. 20A). On day ten, virus-like particles (VLPs) containing Vpx protein were added to the twice-transduced cells. As controls, VLPs lacking Vpx were used, or no VLPs were added. On day fourteen, the percent GFP⁺ cells under each condition was assessed by flow cytometry using standard gating for viable, singlet, lymphoid cells (FIG. 20B). Vpx increased the percentage of GFP⁺ cells, whether vpx was transduced before, or concurrent with, reporter gene transduction, or if Vpx protein was delivered by VLPs after reporter gene transduction (FIG. 16B and FIG. 20C; n=3 biological replicates, p<0.02, 1-way ANOVA with Dunnett post-test). These results suggest that the transduced reporter gene was actively silenced and that vpx overcame reporter silencing.

To confirm that the findings in FIG. 16B were due to effects of vpx on transcriptional silencing of the reporter gene, and not due to effects on transduction efficiency, Jurkat T cells were first transduced with a vector in which the gag-gfp reporter gene was expressed from the SFFV promoter and blasticidin-S deaminase (blasti^{R}) was expressed from the CypA promoter. Four days after transduction with the reporter vector and selection with blasticidin, cells were either challenged with Vpx⁺ VLPs, or transduced and selected with the dual-promoter lentivector encoding vpx and puroR (Lenti 1 in FIG. 20A). Four days later the GFP signal was at background levels unless Vpx was provided, either by VLPs (FIG. 16C) or by vpx transduction (FIG. 16D). The effect of vpx on reporter gene expression was confirmed by qRT-PCR for the reporter mRNA (FIG. 20D). Reporter gene silencing and reactivation by Vpx was not specific to the SFFV promoter since GFP signal was similar when the reporter gene was expressed from the human EEF1A1 (EF1α) promoter or from the Herpes simplex virus type 1 thymidine kinase (TK) promoter (FIG. 20E). These results demonstrate that Vpx overcomes transcriptional silencing of the provirus.

To determine if the ability to activate transcription of silenced proviruses is peculiar to SIV_{MAC}251 Vpx, representative Vpx and Vpr orthologues, selected from across the phylogeny of primate immunodeficiency viruses, were examined. All Vpx proteins tested, SIV_{DRL}D3, SIV_{RCM}NG411, SIV_{AGI}00CM312, SIV_{RCM}02CM8081, SIV_{MND2}5440, HIV-2_{ROD}, SIV_{MAC}251, and SIV_{MNE}027, had transactivating activity in human cells (FIG. 16E and FIG. 20F). Conservation of this activity in human cells among such divergent SIV orthologues was surprising given that SIV_{RCM}NG411 Vpx and SIV_{MND2}5440 Vpx do not degrade human SAMHD1, but they do degrade the SAMHD1 orthologue from their cognate primate host species⁸. Several Vprs from SIVs that lack Vpx, including SIV_{MUS2}CM1246, SIV_{AGM}Ver9063, SIV_{AGM}TAN1, SIV_{MND1}GB1, and SIV_{LST}524, also activated transcription of silent proviral reporters in human cells (FIG. 16E and FIG. 20F). Results could not be obtained from this experimental system concerning the activity of Vprs encoded by SIV_{CPZ}TAN3, HIV-1_{U14788} (Group P), SIV_{GOR}CP684con, HIV-1_{MVP5180} (Group O), HIV-1_{NL4-3} (Group M), SIV_{CPZ}LB7, and SIV_{RCM}02CM8081, presumably because these orthologues caused cell cycle arrest and toxicity (indicated by Ø in FIG. 16E). Vpx and Vpr sequence variability is among the highest observed for lentiviral coding sequences; the sequences shown in FIG. 16E have an average amino acid identity of only 27%. Such diversity likely reflects rapidly evolving, host-pathogen interfaces, and precluded activity predictions based on amino acid sequence conservation to guide the engineering of loss-of-function mutations.

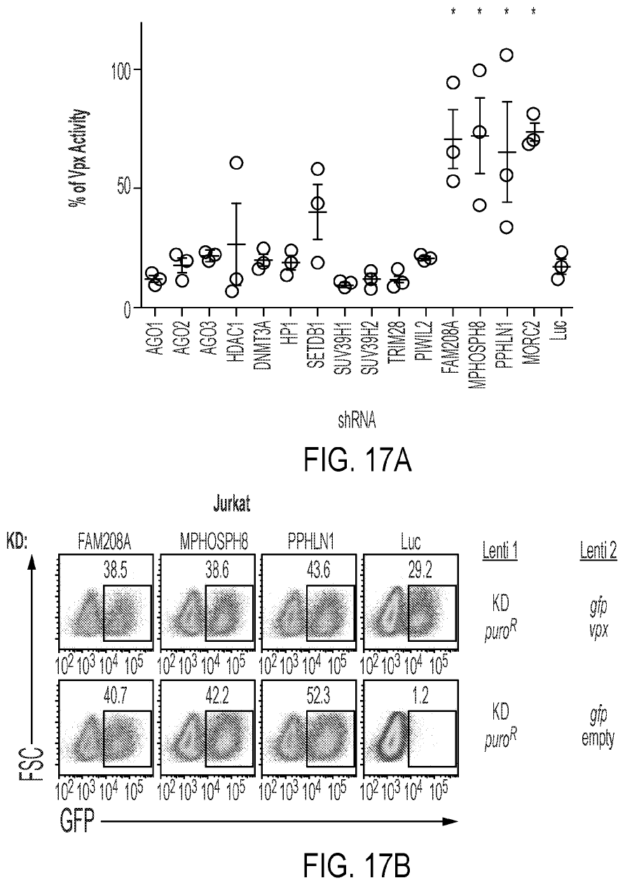

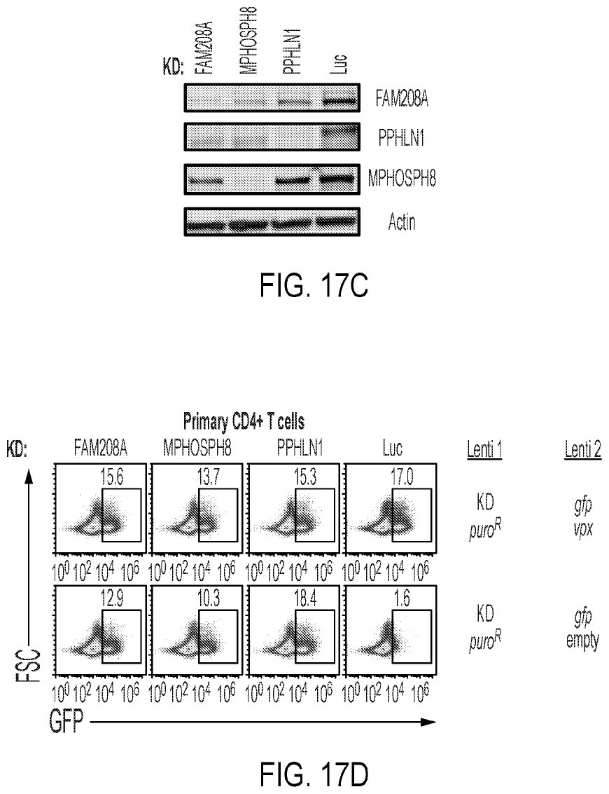

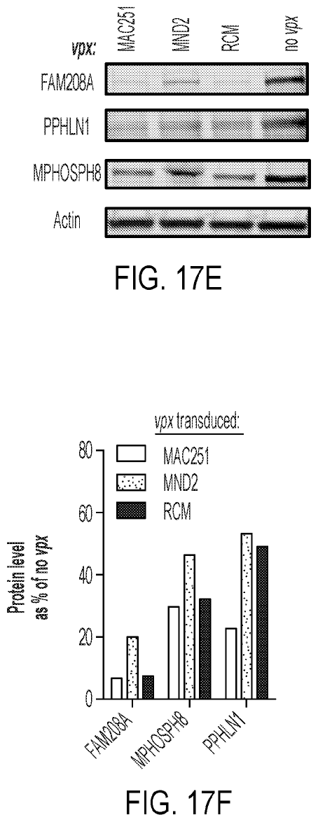

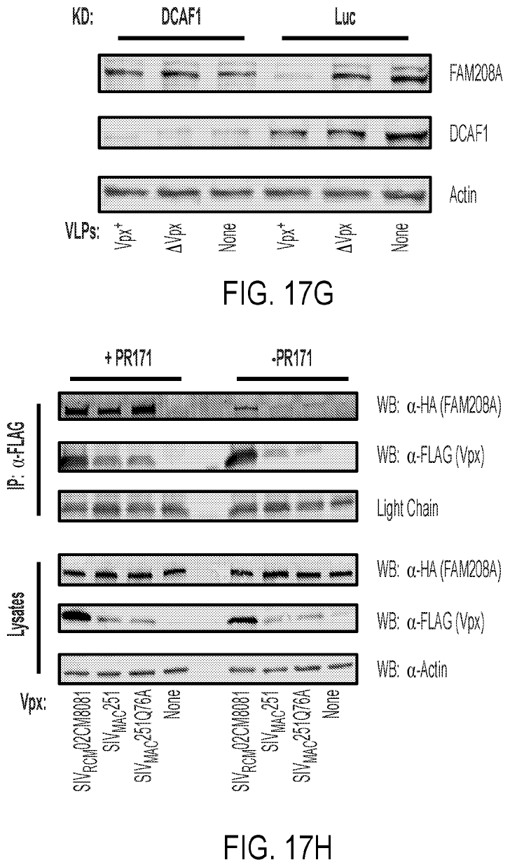

A loss-of-function screen was performed focusing on genes reported to contribute to silencing of retroviruses and other transcriptional targets. Jurkat T cells were transduced with lentivectors that confer puromycin resistance and express shRNAs targeting either AGO1, AGO2, AGO3, DNMT3A, HDAC1, HP1, SUV39H1, SUV39H2, PIWIL2, TRIM28, SETDB1, FAM208A, MPHOSPH8, PPHLN1, or MORC2. After selection for five days with puromycin, cells were transduced with the Lenti 2 gag-gfp reporter vector without vpx (FIG. 20A). Four days later, the change in expression of the gfp reporter due to the knockdowns was calculated as a percentage of the activity observed in a separate population of Jurkat cells transduced to express vpx (FIG. 17A). A given gene was implicated as a transcriptional silencing factor for the provirus reporter gene if the three shRNA targets for that gene differed significantly from that of the luciferase knockdown control (p<0.05, 1-way ANOVA with Dunnett post-test). shRNAs targeting each of the three core components of the Human Silencing Hub (HUSH) complex, FAM208A, MPHOSPH8, and PPHLN1, increased reporter gene expression (FIG. 17A).

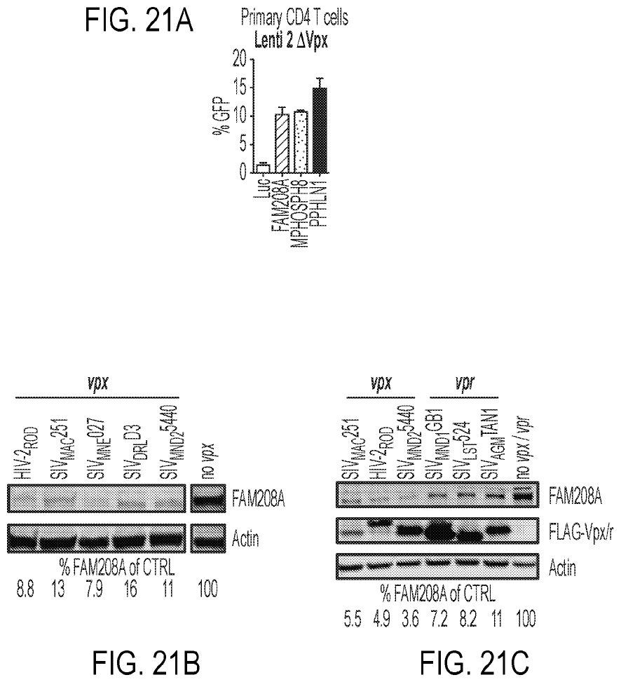

The effect on reporter gene expression in Jurkat T cells of the most effective shRNA target sequences for FAM208A, MPHOSPH8, and PPHLN1 is shown in FIG. 17B. The effectiveness of the knockdown of each of the HUSH complex components in Jurkat cells was confirmed by immunoblotting lysate from these cells with antibodies specific for FAM208A, PPHLN1, or MPHOSPH8 (FIG. 17C). As previously reported, knockdown of any individual HUSH complex component caused a decrease in the level of each of the other components. Similar results on reporter gene expression were obtained when FAM208A, MPHOSPH8, or PPHLN1 were knocked down in primary human CD4⁺ T cells (FIG. 17D). Knockdown of each of the HUSH complex components, then, had the same effect as vpx on lentiviral reporter gene expression (FIGS. 17B and 17D and FIG. 21A). These results demonstrate that the HUSH complex is critical for provirus silencing and raise the possibility that Vpx acts as a substrate adaptor targeting HUSH components to DCAF1 and the CUL4A/B E3 ubiquitin ligase complex for degradation, in the same way that Vpx targets SAMHD1.

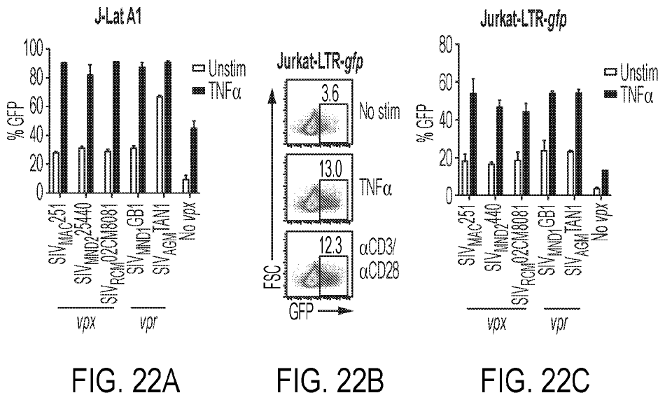

To determine if Vpx promotes the degradation of HUSH complex components, lysate from cells transduced to express SIV_{MAC}251, SIV_{MND2}5440, or SIV_{RCM}NG411 vpx was immunoblotted with antibodies specific for FAM208A, PPHLN1, or MPHOSPH8. All three Vpx proteins reduced the steady-state level of all three core HUSH complex components (FIG. 17E). Among the three HUSH components, though, FAM208A protein levels were decreased more than the other two components (FIG. 17F) so ongoing experiments focused on the effect of Vpx on FAM208A. Indeed, in addition to the three Vpx proteins assessed in FIG. 17E, the other Vpx and Vpr orthologues shown to have transactivation activity in FIG. 16E and FIG. 20F (HIV-2_{ROD} Vpx, SIV_{MNE}027 Vpx, SIV_{DRL}D3 Vpx, SIV_{AGM}TAN1 Vpr, SIV_{MND1}GB1 Vpr, and SIV_{LST}524 Vpr) all decreased the levels of FAM208A (FIGS. 21B and 22C).

To assess whether disruption of FAM208A protein levels by Vpx was dependent upon the DCAF1 adaptor for the CUL4A/B ubiquitin ligase complex, as is the case for SAMHD1, Jurkat T cells were transduced with a lentivector that knocks down DCAF1, or with a control knockdown vector. After selection with puromycin the cells were exposed for 18 hrs to SIV VLPs bearing Vpx, control VLPs that lacked Vpx, or no VLPs. In the DCAF1 knockdown cells, FAM208A protein levels were unchanged by Vpx, indicating that FAM208A disruption by Vpx was dependent upon DCAF1 (FIG. 17G).

Degradation of SAMHD1 requires direct interaction with Vpx or Vpr. To determine if Vpx similarly associates with proteins of the HUSH complex, HA-tagged FAM208A was co-transfected into HEK293 cells with FLAG-tagged SIV_{MAC}251 Vpx or SIV_{RCM}02CM8081 Vpx. When anti-FLAG antibody was used to immunoprecipitate either of the two Vpx proteins from the soluble cell lysate, HA-FAM208A was detected in the immunoprecipitate (FIG. 17H). The strength of the FAM208A signal in the Vpx pull-out increased when the co-transfected HEK293 cells were incubated with the proteasome inhibitor PR171, or when wild-type SIV_{MAC}251 Vpx was replaced in the transfection by a mutant (Q76A) that is incapable of binding DCAF1 (FIG. 17H and FIGS. 21D and 21E). These results demonstrate that FAM208A associates with Vpx and that the interaction results in proteasome-mediated degradation of FAM208A.

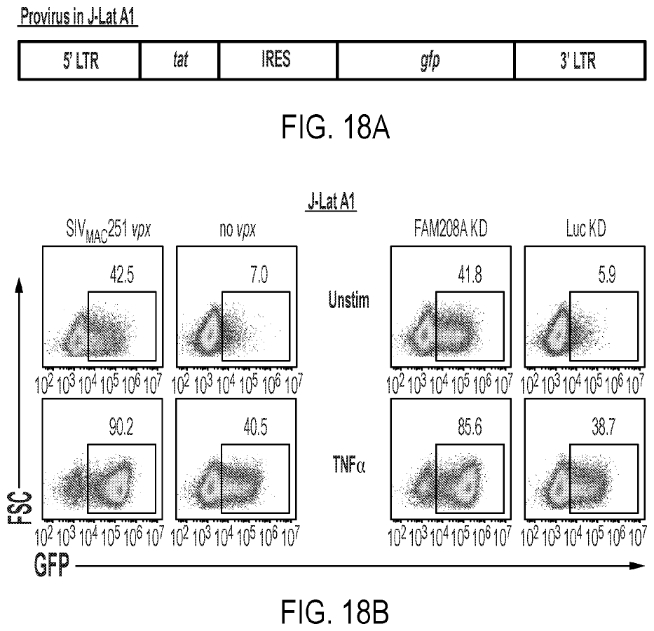

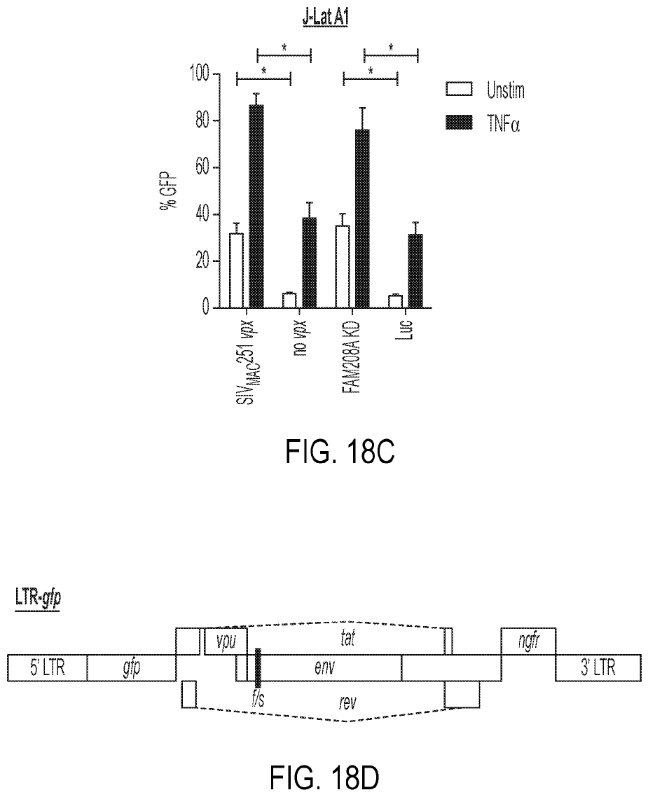

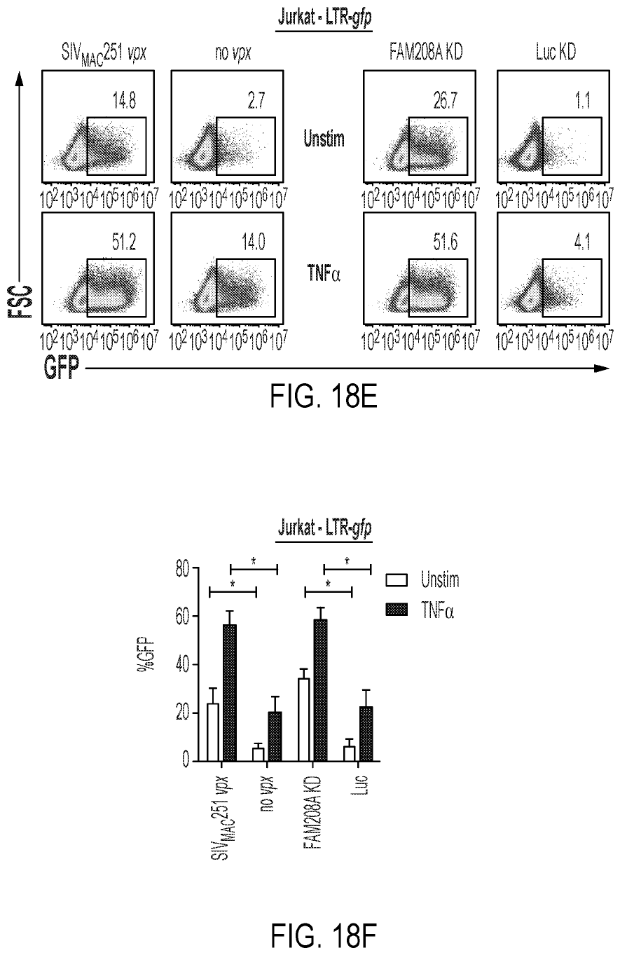

The experiments described above examined the effect of Vpx or Vpr on HIV-1 proviruses in which the reporter gene was transcribed by a heterologous promoter, either human EF1α, HSV TK, or the SFFV LTR (FIGS. 16A to 16E and 17A to 17H and FIGS. 20A to 20F). To determine if Vpx is capable of activating a reporter gene driven by the HIV-1 LTR, the TNFα-responsive, J-Lat A1 clonal cell line was used. In this experimental model of provirus latency, the HIV-1 LTR drives expression of a bicistronic mRNA encoding tat and gfp (FIG. 18A). Transduction with a lentivector expressing SIV_{mac}251 Vpx, or knockdown of FAM208A, caused comparable increase in the percent GFP J-Lat A1 cells, whether the cells were stimulated with TNFα or not (FIGS. 18B and 18C). Transduction of the J-Lat A1 cell line with lentivectors expressing vpx encoded by SIV_{RCM}02CM8081 or SIV_{MND2}5440, as well as with vpr encoded by SIV_{MND1}GB1 or SIV_{AGM}TAN1, caused similar increase in expression of the LTR-driven reporter gene (FIG. 22A).

J-Lat A1 was selected to have a silent HIV-1 LTR-driven provirus with the ability to reactivate in response to TNFα³¹. The unique provirus within a clone such as J-Lat A1 may be sensitive to position-dependent silencing effects and therefore may not accurately reflect the sensitivity of a population of HIV-1 proviruses to transcriptional activation by Vpx or to silencing by FAM208A. To address the effect of Vpx and FAM208A on a population of proviruses with diverse integration sites, Jurkat T cells were transduced with an HIV-1 LTR driven reporter vector (LTR-gfp) that retains complete LTRs, tat, and rev, but has a frameshift mutation in env, an ngfr reporter gene in place of nef, and gfp in place of gag, pol, vif, and vpr (FIG. 18D). Four weeks after transduction with LTR-GFP, the presence of latent proviruses within the pool of Jurkat cells was confirmed by reactivation with either TNFα or TCR-stimulation (FIG. 22B). The Jurkat LTR-gfp cells were then transduced with vectors expressing SIV_{MAC}251 Vpx or shRNA targeting FAM208A, and selected with puromycin. Compared with control cells, vpx or FAM208A knockdown increased the percentage of GFP cells, whether cells were treated with TNFα or not (FIGS. 18E and 18F). Similar results were obtained in three independently generated biological replicate experiments, in which vpx was delivered or FAM208A was knocked down, from four to eight weeks after the first LTR-GFP transduction (FIG. 18F). Additionally, expression vectors for SIV_{MND2}5440 Vpx, SIV_{RCM}02CM8081 Vpx, SIV_{MND1}GB1 Vpr, or SIV_{AGM}TAN1 Vpr all increased GFP expression in Jurkat LTR-gfp cells (FIG. 22C). Together, these experiments demonstrate that FAM208A contributes to the transcriptional repression of clonal or polyclonal LTR reporter lines, and that primate immunodeficiency viruses counteract this activity via their Vpx and Vpr proteins.

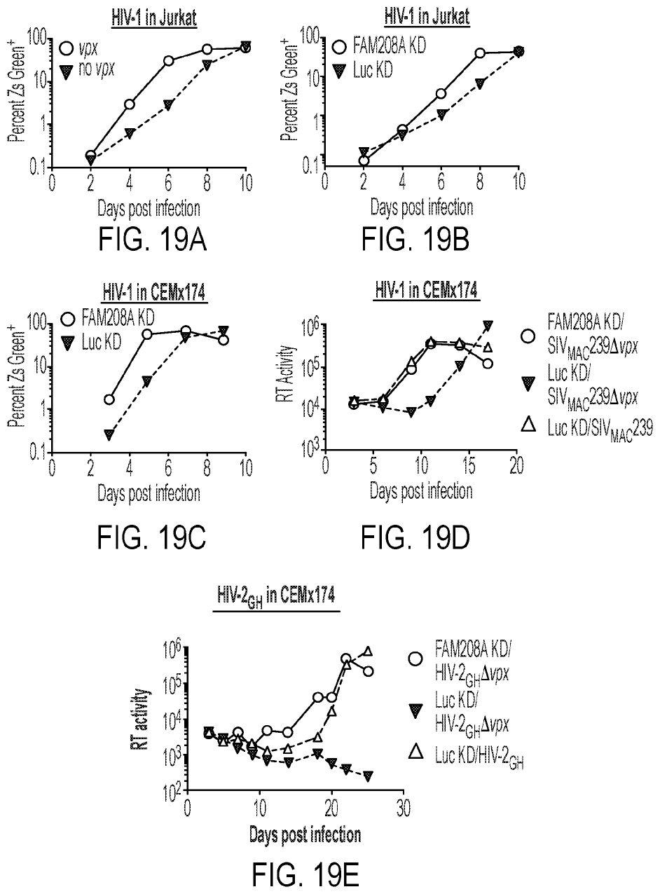

The effect of Vpx or FAM208A knockdown on spreading infection with replication-competent primate immunodeficiency viruses was tested next. Jurkat T cells transduced to express SIV_{MAC}251 vpx, or cells transduced with control vector, were infected with HIV-1-ZsGreen, a replication-competent HIV-1_{NL4-3} clone, that encodes ZsGreen in place of nef (Table 1). Infection was monitored by determining the percent ZsGreen⁺ cells with flow cytometry, every two days for ten days. Compared with the control, HIV-1 replication kinetics was accelerated by vpx (FIG. 19A). In similar fashion, HIV-1 infection of Jurkat cells transduced with the FAM208A knockdown vector resulted in faster replication kinetics (FIG. 19B).

HIV-1 vpr has no detectable effect on HIV-1 replication in tissue culture spreading infections with dividing target cells. This is presumably related to the cell cycle arrest toxicity, and selection against vpr in tissue culture, since the effects of vpr on HIV-1 are evident when proviral expression is restricted to single cycle infection or cells are arrested with aphidicolin. Nonetheless, vpr offers a selective advantage in vivo since cloned vpr mutant virus was repaired when virus was injected into replication permissive chimps, or in an infected person.

SIV_{MAC}239 does not replicate in Jurkat cells so CEMx174 cells were used to test the effect of FAM208A and vpx on replication of this virus. As in Jurkat cells, FAM208A knockdown increased HIV-1 replication kinetics in CEMx174 cells (FIG. 19C). Then, CEMx174 cells transduced with FAM208A or control knockdown vectors were challenged with SIV_{MAC}239 or SIV_{MAC}239-Δvpx and replication was assessed by measuring reverse transcriptase activity in the supernatant. In the absence of vpx, SIV_{MAC}239 replicated slower than the wild-type virus in control knockdown CEMx174 cells (FIG. 19D). This delay in SIV_{MAC}239-Δvpx replication kinetics was not observed when FAM208A was knocked down (FIG. 19D). Replication of HIV-2_{GH}Δvpx was undetectable in control knockdown CEMx174 cells (FIG. 19E). However, FAM208A knockdown rescued the replication of HIV-2_{GH}Δvpx to the level of wild-type HIV-2_{GH} in control cells (FIG. 19E). These experiments indicate that FAM208A inhibits primate immunodeficiency virus replication and that Vpx antagonizes this restriction, resulting in expression—or increased expression—from integrated proviruses, permitting virus spread.

The experiments reported here demonstrated that vpx and vpr activate transcription from silenced proviruses and that this activity was mimicked by knockdown of each of the HUSH complex components. These two observations were then shown to be linked by the finding that Vpx associated with, and promoted degradation of HUSH complex protein FAM208A, in a DCAF1- and proteasome-dependent manner. Latent provirus activation and human FAM208A degradation were exhibited by a broader range of primate immunodeficiency vpx and vpr orthologues than are capable of degrading human SAMHD1, perhaps due to the greater conservation and essential nature of FAM208A. Vpx and FAM208A disruption were important for transcriptional activation of latent HIV-1 provirus pools and for the ability of HIV-1, HIV-2, and SIV_{MAC} to effectively spread through cultured CD4⁺ T cells. Further understanding of the contributions of Vpx and Vpr and of the HUSH complex proteins, in concert with other transcriptional silencing mechanisms targeting HIV-1, is hoped to inform ongoing efforts to control or eliminate proviruses in HIV-1 infected patients.

### Methods

### Data Reporting

No statistical methods were used to predetermine sample size. The experiments were not randomized. The investigators were not blinded to allocation during experiments and outcome assessment.

### Plasmids

Sequences encoding 3×FLAG N-terminal-tagged Vpx and Vpr proteins were ordered as codon-optimized, gBlocks Gene Fragments (Integrated DNA Technologies; <www.idtdna.com/>) and cloned into either the pscALPS vector for transduction, or into pcDNA3.1 for transfection. pAPM-D4 is a truncated derivative of the pAPM lentivector that expresses the puromycin acetyltransferase and miR30-based shRNA from the SFFV promoter. Table 1 lists all plasmids used here, with corresponding addgene accession numbers, target sites used in particular knockdown vectors, and accession numbers for all the Vpx and Vpr orthologues tested here.

### Cell Culture

Cells were cultured at 37° C. in 5% CO₂ humidified incubators and monitored for mycoplasma contamination using the Mycoplasma Detection kit (Lonza LT07-318). HEK293 cells (ATCC) were used for viral production and were maintained in DMEM supplemented with 10% FBS, 20 mM L-glutamine (ThermoFisher), 25 mM HEPES pH 7.2 (SigmaAldrich), 1 mM sodium pyruvate (ThermoFisher), and 1×MEM non-essential amino acids (ThermoFisher). Jurkat and CEMx174 cells (ATCC) were cultured in RPMI-1640 supplemented with 10% heat inactivated FBS, 20 mM L-glutamine, 25 mM HEPES pH 7.2, 1 mM sodium pyruvate, 1×MEM non-essential amino acids and Pen/Strep (ThermoFischer) (RPMI-FBS complete). J-Lat A1 cells⁻ (NIH AIDS Reagent Program, catalogue #9852, donated by Eric Verdin) were cultured in RPMI-FBS complete media.

Leukopaks were obtained from anonymous, healthy, blood bank donors (New York Biologics, Southhampton, N.Y.). As per NIH guidelines (<grants.nih.gov/grants/policy/hs/faqs_aps_definitions.htm>), experiments with these cells were declared non-human subjects research by the University of Massachusetts Medical School Institutional Review Board. PBMCs were isolated from leukopaks by gradient centrifugation on Histopaque-1077 (Sigma-Aldrich). CD4⁺ T cells were enriched from PBMCs using anti-CD4 microbeads (Miltenyi) and were >95% CD4⁺. CD4⁺ T cells were cultured in RPMI-FBS complete media in the presence of 50 U/mL hIL-2 (NIH AIDS Reagent Program, catalogue #136).

### Vector Production

HEK293 cells were seeded at 75% confluency in 6-well plates and transfected with 6.25 μL Transit LT1 lipid reagent (Mirus) in 250 μL Opti-MEM (Gibco) with 2.25 μg total plasmid DNA. Full replicating virus was produced by transfection of 2.25m of the indicated plasmid. Lenti-GFP reporters, LTR-GFP reporter, and shRNA lentivectors were produced by transfection of the lentivector, psPAX2 gagpol expression plasmid, and the pMD2.G VSV G expression plasmid, at a DNA ratio of 4:3:1. Vpx containing SIV-VLPs were produced by transfection at a 7:1 plasmid ratio of SIV3+ to pMD2.G, and ΔVpx SIV VLPs were produced the same way using SIV3+ ΔVpx plasmid. 12 hrs after transfection, media was changed to the specific media for the cells that were to be transduced. Viral supernatant was harvested 2 days later, filtered through a 0.45 μm filter, and stored at 4° C.

### Reverse Transcriptase Assay

Virions in the transfection supernatant were quantified by a PCR-based assay for reverse transcriptase activity³⁰. 5 μl transfection supernatant were lysed in 5 μL 0.25% Triton X-100, 50 mM KCl, 100 mM Tris-HCl pH 7.4, and 0.4 U/μl RNase inhibitor (RiboLock, ThermoFisher). Viral lysate was then diluted 1:100 in a buffer of 5 mM (NH₄)₂SO₄, 20 mM KCl, and 20 mM Tris-HCl pH 8.3. 10 μL was then added to a single-step, RT PCR assay with 35 nM MS2 RNA (IDT) as template, 500 nM of each primer (5′-TCCTGCTCAACTTCCTGTCGAG-3′ (SEQ ID NO: 12) and 5′-CACAGGTCAAACCTCCTAGGAATG-3′ (SEQ ID NO: 13)), and hot-start Taq (Promega) in a buffer of 20 mM Tris-Cl pH 8.3, 5 mM (NH₄)₂SO₄, 20 mM KCl, 5 mM MgCl₂, 0.1 mg/ml BSA, 1/20,000 SYBR Green I (Invitrogen), and 200 μM dNTPs. The RT-PCR reaction was carried out in a Biorad CFX96 cycler with the following parameters: 42° C. 20 min, 95° C. 2 min, and 40 cycles [95° C. for 5 s, 60° C. 5 s, 72° C. for 15 s and acquisition at 80° C. for 5 s]. 3 part vector transfections typically yielded 10⁶ RT units/μL.

### Transductions

For generating pools of shRNA knockdown Jurkat and CEMx174 lines, cells were plated at 10⁶ cells/mL in RPMI-FBS complete and transduced with 10⁷ RT units of viral vector per 10⁶ cells, followed by selection with 1 μg/ml puromycin (InvivoGen, cat #ant-pr-1). To generate stable gag-gfp expressing Jurkat cells, cells were transduced as for shRNA KD above, followed by selection with 5 μg/mL blasticidin (InvivoGen, cat #ant-bl-1) at day 3 after transduction.

CD4⁺ T cells were stimulated in RPMI-FBS complete, with 50 U/ml IL-2 and 5 μg/mL PHA-P (Sigma, cat #L-1668). After 3 days, T cells were washed and replated at 3×10⁶ cells/mL in RPMI-FBS complete, with 50 U/ml IL-2. Cells were transduced with 10⁸ RT units of viral vector per 10⁶ cells followed by selection in 2 μg/mL puromycin. After selection, cells were re-plated in RPMI-FBS complete with 50 U/ml IL-2 at 3×10⁶ cells/mL in RPMI-FBS complete and transduced again with the indicated GFP vectors, 10⁸ RT units of viral vector per 10⁶ cells. Transduced T cells were analyzed 4-5 days after the 2nd transduction.

### Lentiviral Infections

5×10⁵ Jurkat or CEMx174 cells were incubated with 5×107 RT units of HIV-1NL4.3, HIV-2GH, HIV-2GHΔvpx, SIVMAC239, or SIVMAC239Δvpx virus stocks produced in HEK-293 cells for 12 hrs in RPMI-FBS complete media, followed by a wash in media and replated in 1 mL of media. Cells were split every 2-3 days and analyzed. For monitoring of HIV-1 ZsGreen infection, when cells were split, aliquots were fixed in BD Cytofix followed by analysis of GFP+ cells by flow cytometry to determine infection levels. For monitoring of SIV and HIV-2 infections, 50 μL aliquots of supernatant were analyzed for RT activity using the above described RT assay.

### Re-Activation Assays

LTR-driven GFP re-activation assays were performed with 10 ng/ml hTNFα (Invivogen, cat #rcyc-htnf), or with 1m/m1 soluble α-CD3 and α-CD28 antibody. α-CD3 antibody (clone OKT3) and α-CD28 antibody (clone CD28.2) were provided by Lisa Cavacini (MassBiologics, Mattapan, Mass.).

qRT-PCR

Total RNA was isolated from Jurkat cells using Trizol reagent followed by purification of RNA with RNeasy Plus Mini (Qiagen) with Turbo DNase (ThermoFisher) in order to limit DNA contamination. First-strand synthesis used Superscript III Vilo Master mix (Invitrogen) with random hexamers. qPCR was performed in 20 μL using SYBR green reagent (Applied Biosystems) with primers designed against gag, gfp, and gapdh for normalization. Amplification was on a CFX96 Real Time Thermal Cycler (Bio-Rad) using the following program: 95° C. for 10 min, then 45 cycles of 95° C. for 15 s and 60° C. for 60 s. Cells not transduced with Lenti-GFP vector were used as negative control and the housekeeping gene GAPDH was used to normalize expression levels. The primer sequences used were:

gag primers
(Forward: 5′-GCTGGAAATGTGGAAAGGAA-3′, SEQ ID NO: 4;
Reverse: 5′-AGTCTCTTCGCCAAACCTGA-3′, SEQ ID NO: 5),
gfp primers
(Forward: 5′-GCAGAGGTGAAGTTCGAAGG-3′, SEQ ID NO: 6;
Reverse: 5′-CCAATTGGTGTGTTCTGCTG-3′, SEQ ID NO: 7),
gapdh primers
(Forward: 5′-AGGGCTGCTTTTAACTCTGGT-3′,
SEQ ID NO: 8;
Reverse: 5′-CCCCACTTGATTTTGGAGGGA-3′,
SEQ ID NO: 9).

### Flow Cytometry

Cells were fixed in BD Cytofix Buffer prior to data acquisition on a BD C6 Accuri. Data was analyzed in FlowJo.

### Western Blot

Cells were washed in PBS, counted, normalized for cell number, and lysed directly in 1×SDS-PAGE sample buffer. Samples were run on NuPage 4-12% Bis-Tris gels followed by blotting onto nitrocellulose membranes. Primary antibodies used: FAM208A (Atlas, HPA00875), MPHOSPH8 (Proteintech, 16796-1-AP), PPHLN1 (Sigma, HPA038902), SETDB1 (Proteintech 11231-1-AP), DCAF1 (Proteintech, 11612-1-AP), FLAG (Novus, NB600-345), FLAG (Sigma, F1804, used for IP), and HA (Biolegend, 901501).

### Vpr and Vpx Phylogeny

The following Vpr and Vpx amino acid sequence alignments were obtained from the Los Alamos National Laboratories (LANL) HIV sequence database: 2016 HIV-1/SIV_{CPZ} Vpr, 2016 HIV-2/SIVsmm Vpr, 2016 HIV-2/SIVsmm Vpx, 2016 other SIV Vpr, and 2016 other Vpx. Consensus sequences were generated for HIV-1 group M subtypes A, B, C, D, F, G, H, I, J, and those designated U in the LANL database, as well as group N. A master alignment was scaffolded from the above alignments and re-aligned by hand. Redundant SIV and HIV-2 Vpr and Vpx sequences were removed, and the sequences of individual HIV-1 isolates were replaced with the consensus sequences. This was used to generate a master phylogeny using RAxML 8.2.11, as implemented in Geneious with gamma LG substitution model and Rapid Bootstrapping with search for best scoring tree algorithm. This master tree was utilized to identify major relationships and identify a reduced number of sequences to retain while maintaining the overall phylogenic structure. Vpx and Vpr sequences from the following viral isolates were retained: HQ179987, L20571, M15390, AF208027, AB731738, KP890355, M15390, AF208027, AB731738, KP890355, U58991, M30931, L40990, KJ461715, AF301156, U42720, AY169968, DQ373065, DQ373064, DQ374658, FJ919724, AJ580407, KM378563, KM378563, FJ424871, M66437, AF468659, AF468658, AF188116, M76764, LC114462, M27470, AY159322, AY159322, U79412, U79412, AY340701, AY340700, EF070329, KF304707, FM165200, HM803690, HM803689, AF382829, AF349680, HM803690, HM803689, AF349680, U04005, JX860432, JX860430, JX860426, JX860432, M83293, M83293, AF131870, AY523867, AM182197, AM713177, U26942, and the HIV-1 group M Glade B consensus. These sequences were used to generate a phylogeny using the same method as above. Superfluous taxa were pruned from this phylogeny using Mesquite 3.4 and the resulting tree was visualized in FigTree v1.4.3.

### Sampling

At least three biological replicates were performed for all experiments. The screen for factors mediating silencing of the Lenti-GFP vector utilized 3 target sequences for each candidate gene. Flow cytometry plots in the figures show representative data taken from experiments performed at the same time. HIV-1, HIV-2, and SIV spreading experiments were repeated 3 times each and representative data of one such experiment is shown.

### Statistics

Information regarding the statistical tests utilized, and the n values, are found in the figure legends. Statistical analysis of the knockdown screen of factors involved in silencing of Lenti-GFP was analyzed by one-way ANOVA with Dunnett post test comparing 3 shRNA target sites to control knockdown conditions. All statistics presented were performed using PRISM 5.0 (GraphPAD Software, La Jolla, Calif.).

TABLE 1
Plasmids used.

| Plasmid Name | Purpose | Notes |
|---|---|---|
| HIV1- | Replication | HIV-1 NL4-3 in pBluescript with flanking host sequences |
| ZsGreen | competent HIV-1 | deleted. ZsGreen in place of nef |
| SIV_{mac}239 | Replication | Molecular clone of SIV_{MAC239} proviral DNA |
| SpX | competent SIV | |
| SIV_{mac}239 | SIV Δvpx | Molecular clone of SIV_{MAC239}Δvpx proviral DNA |
| SpX ΔVpx | | |
| pGL-AN | Replication | Molecular clone of HIV-2 |
| | competent HIV-2 | |
| pGL-St | HIV-2 ΔVpx full | Molecular clone of HIV-2 with disruption of vpx ORF |
| | length wt | |
| pMD2.G | VSV G | Pseudotype HIV-1 vectors with VSV Glycoprotein |
| psPAX2 | HIV-1 gag-pol | Encodes gag structural proteins and pol enzymes to |
| | | generate virion particles |
| SIV3+ | SIV_{MAC251} gag- | Production of SIV VLPs containing Vpx protein |
| | pol/vpx | |
| SIV3+ Δvpx | SIV_{MAC251} gag- | Production of SIV VLPs without Vpx protein. |
| | pol/Δvpx | |
| pscALPS | Lenti-gfp-blasti | SFFV promoter expresses gag-gfp fusion with CypA |
| gag-gfp/blasti | | promoter driving blasticidin resistance gene |
| pscALPS | Lenti-gfp-vpx | SFFV promoter expresses gag-gfp fusion with CypA |
| gag-gfp/vpx | | promoter driving expression of SIV_{MAC}251 vpx |
| pscALPS | Lenti-gfp-Δvpx | SFFV promoter expresses gag-gfp fusion with no ORF after |
| gag-gfp/Δvpx | | CypA promoter |
| pecALPS | Lenti-gfp-blasti | EIF1a promoter expresses gag-gfp fusion and CypA |
| gag-gfp/blasti | | promoter expresses blasticidin resistance gene |
| pkcALPS- | Lenti-gfp-blasti | TK promoter expresses gag-gfp fusion and CypA promoter |
| gag-gfp/blasti | | expresses blasticidin resistance gene |
| HIV-1 LTR- | HIV-1 LTR-gfp | HIV-1 LTR driven reporter vector that retains complete |
| gfp | | LTRs, tat, and rev, but has a frameshift mutation in env, an |
| | | ngfr reporter gene in place of nef, and gfp in place of gag, |
| | | pol, vif, and vpr |
| pscALPS- | Lentivector | Encodes codon optimized 3xFLAG-Vpx_{MAC}251 and |
| SIV_{MAC}251 vpx | expressing vpx | puromycin resistance protein |
| pscALPS | Lentivector | Encodes codon optimized 3xFLAG-SIV_{MND2} Vpx |
| SIV_{MND2} vpx | expressing vpx | (AY159322) and puromycin resistance protein |
| pscALPS- | Lentivector | Encodes codon optimized 3xFLAG-SIV_{RCM} Vpx |
| SIV_{RCM} vpx | expressing vpx | (AF349680) and puromycin resistance protein |
| pscALPS- | Lentivector | Encodes codon optimized 3xFLAG-SIV_{MNE} Vpx (U79412) |
| SIV_{MNE} vpx | expressing vpx | and puromycin resistance protein |
| pscALPS- | Lentivector | Encodes codon optimized 3xFLAG-SIV_{DRL} Vpx |
| SIV_{DRL} vpx | expressing vpx | (KM378563) and puromycin resistance protein |
| pscALPS- | Lentivector | Encodes codon optimized 3xFLAG-SIV_{AGI} Vpx |
| SIV_{AGI} vpx | expressing vpx | (HM803690) and puromycin resistance protein |
| pscALPS- | Lentivector | Encodes codon optimized 3xFLAG-HIV2_{ROD} Vpx |
| HIV2_{ROD} vpx | expressing vpx | (M15390) and puromycin resistance protein |
| pscALPS- | Lentivector | Encodes codon optimized 3xFLAG-SIV_{MND1} Vpr (M27470) |
| SIV_{MND1} vpr | expressing vpx | and puromycin resistance protein |
| pscALPS- | Lentivector | Encodes codon optimized 3xFLAG-SIV_{LST} Vpr |
| SIV_{LST} vpr | expressing vpx | (AF188116) and puromycin resistance protein |
| pscALPS- | Lentivector | Encodes codon optimized 3xFLAG-SIV_{AGM} Vpr (TAN1) |
| SIV_{AGM}TAN1 vpr | expressing vpx | (U58991) with repaired premature stop codon and |
| | | puromycin resistance protein |
| pscALPS- | Lentivector | Encodes codon optimized 3xFLAG-SIV_{TAL} Vpr |
| SIV_{TAL} vpx | expressing vpx | (AM182197) and puromycin resistance protein |
| pscALPS- | Lentivector | Encodes codon optimized 3xFLAG-SIV_{SYK} Vpr (L06042) |
| SIV_{SYK} vpx | expressing vpx | and puromycin resistance protein |
| pscALPS- | Lentivector | Encodes codon optimized 3xFLAG-SIV_{SAB} Vpr (U04005) |
| SIV_{SAB} vpx | expressing vpx | and puromycin resistance protein |
| pscALPS- | Lentivector | Encodes codon optimized 3xFLAG-SIV_{DEB} Vpr (FJ919724) |
| SIV_{DEB} vpx | expressing vpx | and puromycin resistance protein |
| pscALPS- | Lentivector | Encodes codon optimized 3xFLAG-SIV_{ASC} Vpr |
| SIV_{ASC} vpx | expressing vpx | (KJ461715) and puromycin resistance protein |
| pscALPS- | Lentivector | Encodes codon optimized 3xFLAG-SIV_{AGM} Vpr (Ver |
| SIV_{AGM}VER9063 vpr | expressing vpx | 9063) (L40990) and Puro resistance marker |
| pscALPS- | Lentivector | Encodes codon optimized 3xFLAG-SIV_{AGM} Vpr (Ver |
| SIV_{AGM}VER | expressing vpx | AGM3) (M30931) and puromycin resistance protein |
| AGM3 vpr | | |
| pscALPS- | Lentivector | Encodes codon optimized 3xFLAG-SIV_{GRV} Vpr (M66437) |
| SIV_{GRV} vpr | expressing vpx | and puromycin resistance protein |
| pscALPS- | Lentivector | Encodes codon optimized 3xFLAG-SIV_{AGM} Vpr |
| SIV_{AGM}MAL vpr | expressing vpx | (MAL_ZMB) (LC114462) and puromycin resistance protein |
| pscALPS- | Lentivector | Encodes codon optimized 3xFLAG SIV_{DEN} Vpr |
| SIV_{DEN} vpr | expressing vpx | (AJ580407) and puromycin resistance protein |
| pscALPS- | Lentivector | Encodes codon optimized 3xFLAG SIV_{gsn-cn71} Vpr |
| SIV_{GSN}CN71 vpr | expressing vpx | (AF468658) and puromycin resistance protein |
| pscALPS- | Lentivector | Encodes codon optimized 3xFLAG SIV_{GSN-CN166} Vpr |
| SIV_{GSN}CN166 vpr | expressing vpx | (AF468659) and puromycin resistance protein |
| pscALPS- | Lentivector | Encodes codon optimized 3xFLAG-SIV_{SUN} Vpr |
| SIV_{SUN} vpr | expressing vpx | (AF131870) and puromycin resistance protein |
| pscALPS- | Lentivector | Encodes codon optimized 3xFLAG-SIV_{CPZ-TAN3} Vpr |
| SIV_{CPZ}TAN3 vpr | expressing vpx | (DQ374658) and puromycin resistance protein |
| pscALPS- | Lentivector | Encodes codon optimized 3xFLAG-SIV_{CPZ-LB7} Vpr |
| SIV_{CPZ}LB7 vpr | expressing vpx | (DQ373064) and puromycin resistance protein |
| pscALPS- | Lentivector | Encodes codon optimized 3xFLAG-SIV_{GOR} Vpr (FJ424871) |
| SIV_{GOR} -vpr | expressing vpx | and puromycin resistance protein |
| pscALPS- | Lentivector | Encodes codon optimized 3xFLAG-HIV1 Group O Vpr |
| HIV-1 Group | expressing vpx | (L20571) and puromycin resistance protein |
| O-vpr | | |
| pscALPS- | Lentivector | Encodes codon optimized 3xFLAG-HIV1 Group P Vpr |
| HIV-1 Group | expressing vpx | (HQ179987) and puromycin resistance protein |
| P-vpr | | |
| pscALPS- | Lentivector | Encodes codon optimized 3xFLAG-SIV_{RCM} Vpr |
| SIV_{RCM-}vpr | expressing vpx | (HM803689) and puromycin resistance protein |
| pscALPS- | Lentivector | Encodes codon optimized 3xFLAG-SIV_{WRC} Vpr |
| SIV_{WRC-}vpr | expressing vpx | (AM713177) and puromycin resistance protein |
| pscALPS- | Lentivector | Encodes codon optimized 3xFLAG-SIV_{MUS2}1246 Vpr |
| SIV_{MUS2}1246-vpr | expressing vpx | (EF070329) and puromycin resistance protein |
| pcDNA3.1 | Expression | Encodes codon optimized FLAG tagged SIV_{MAC}251-Vpx |
| FLAG- | plasmid | |
| SIV_{MAC}251- | | |
| Vpx | | |
| pcDNA3.1 | Expression | Encodes codon optimized FLAG tagged SIV_{MAC}251-Vpx- |
| FLAG- | plasmid | Q76A mutant |
| SIV_{MAC}251- | | |
| Vpx-Q76A | | |
| pcDNA3.1 | Expression | Encodes codon optimized FLAG tagged SIV_{RCM}-Vpx |
| FLAG- | plasmid | |
| SIV_{RCM-}Vpx | | |
| pcDNA3.1 | Expression | Encodes codon optimized HA tagged FAM208A |
| HA- | plasmid | |
| FAM208A | | |

TABLE 2
pAPM-D4 Plasmids used.

| Plasmid | | |
|---|---|---|
| Name | Purpose | Notes |
| pAPM-D4- | Lentivector | SFFV promoter expressing puromycin resistance protein |
| miR30-L1221 | luciferase | and miR30-shRNA target site: 5′- |
| | knockdown | CTTGTCGATGAGAGCGTTTGT-3′ (SEQ ID NO: 14); |
| | | negative control for other knockdowns |
| pAPM-D4 | Lentivector | SFFV promoter expressing puromycin resistance protein |
| miR30- | HDAC1 | and miR30-shRNA target site: 5′- |
| HDAC1 ts1 | knockdown | TATGAGTCATGCGGATTCG-3′ (SEQ ID NO: 15) |
| pAPM-D4 | Lentivector | SFFV promoter expressing puromycin resistance protein |
| miR30- | HDAC1 | and miR30-shRNA target site: 5′- |
| HDAC1 ts2 | knockdown | TAAGAACGGGAAGAATGGG-3′ (SEQ ID NO: 16) |
| pAPM-D4 | Lentivector | SFFV promoter expressing puromycin resistance protein |
| miR30- | HDAC1 | and miR30-shRNA target site: 5′- |
| HDAC1 ts3 | knockdown | TTAATGTAGTCATCGCTGT-3′ (SEQ ID NO: 17) |
| pAPM-D4 | Lentivector | SFFV promoter expressing puromycin resistance protein |
| miR30-AGO1 | AGO1 | and miR30-shRNA target site: 5′- |
| ts1 | knockdown | TTCTGCTTGAAATACTGTG-3′ (SEQ ID NO: 18) |
| pAPM-D4 | Lentivector | SFFV promoter expressing puromycin resistance protein |
| miR30-AGO1 | AGO1 | and miR30-shRNA target site: 5′- |
| ts2 | knockdown | TGATATCAGAGATTTCTGG-3′ (SEQ ID NO: 19) |
| pAPM-D4 | Lentivector | SFFV promoter expressing puromycin resistance protein |
| miR30-AGO1 | AGO1 | and miR30-shRNA target site: 5′- |
| ts3 | knockdown | TTGACATTGATCTTGAGGC-3′ (SEQ ID NO: 20) |
| pAPM-D4 | Lentivector | SFFV promoter expressing puromycin resistance protein |
| miR30-AGO2 | AGO2 | and miR30-shRNA target site: 5′- |
| ts1 | knockdown | TAATACATCTTTGTCCTGC-3′ (SEQ ID NO: 21) |
| pAPM-D4 | Lentivector | SFFV promoter expressing puromycin resistance protein |
| miR30-AGO2 | AGO2 | and miR30-shRNA target site: 5′- |
| ts2 | knockdown | TCATCTGCACGCACTGCGT-3′ (SEQ ID NO: 22) |
| pAPM-D4 | Lentivector | SFFV promoter expressing puromycin resistance protein |
| miR30-AGO2 | AGO2 | and miR30-shRNA target site: 5′- |
| ts3 | knockdown | TTGCTAATCTCTTCTTGCC-3′ (SEQ ID NO: 23) |
| pAPM-D4 | Lentivector | SFFV promoter expressing puromycin resistance protein |
| miR30-AGO3 | AGO3 | and miR30-shRNA target site: 5′- |
| ts1 | knockdown | TGACTTGAACACATTGTGT-3′ (SEQ ID NO: 24) |
| pAPM-D4 | Lentivector | SFFV promoter expressing puromycin resistance protein |
| miR30-AGO3 | AGO3 | and miR30-shRNA target site: 5′- |
| ts2 | knockdown | TCTGAACTACAATGTAGGT-3′ (SEQ ID NO: 25) |
| pAPM-D4 | Lentivector | SFFV promoter expressing puromycin resistance protein |
| miR30-AGO3 | AGO3 | and miR30-shRNA target site: 5′- |
| ts3 | knockdown | TAGCTTCTTGATACATCGT-3′ (SEQ ID NO: 26) |
| pAPM-D4 | Lentivector | SFFV promoter expressing puromycin resistance protein |
| miR30- | SETDB1 | and miR30-shRNA target site: 5′- |
| SETDB1 ts1 | knockdown | TTCGCATGCTGACTATCAG-3′ (SEQ ID NO: 27) |
| pAPM-D4 | Lentivector | SFFV promoter expressing puromycin resistance protein |
| miR30- | SETDB1 | and miR30-shRNA target site: 5′- |
| SETDB1 ts2 | knockdown | ACACAATCCATCTTCTCCA-3′ (SEQ ID NO: 28) |
| pAPM-D4 | Lentivector | SFFV promoter expressing puromycin resistance protein |
| miR30- | SETDB1 | and miR30-shRNA target site: 5′- |
| SETDB1 ts3 | knockdown | TTGTTGTCAAATTTCACCT-3′ (SEQ ID NO: 29) |
| pAPM-D4 | Lentivector | SFFV promoter expressing puromycin resistance protein |
| miR30- | TRIM28 | and miR30-shRNA target site: 5′- |
| TRIM28 ts1 | knockdown | AAGGTTGTAGTCCTCAGTG-3′ (SEQ ID NO: 30) |
| pAPM-D4 | Lentivector | SFFV promoter expressing puromycin resistance protein |
| miR30- | TRIM28 | and miR30-shRNA target site: 5′- |
| TRIM28 ts2 | knockdown | TCAATAACAATAAGGTTGT-3′ (SEQ ID NO: 31) |
| pAPM-D4 | Lentivector | SFFV promoter expressing puromycin resistance protein |
| miR30- | TRIM28 | and miR30-shRNA target site: 5′- |
| TRIM28 ts3 | knockdown | TGAGTAGGGATCATCTCCT-3′ (SEQ ID NO: 32) |
| pAPM-D4 | Lentivector | SFFV promoter expressing puromycin resistance protein |
| miR30- | DNMT3a | and miR30-shRNA target site: 5′- |
| DNMT3a ts1 | knockdown | TAATCTCCTTGACCTTGGG-3′ (SEQ ID NO: 33) |
| pAPM-D4 | Lentivector | SFFV promoter expressing puromycin resistance protein |
| miR30- | DNMT3a | and miR30-shRNA target site: 5′- |
| DNMT3a ts2 | knockdown | TATCATTCACAGTGGATGC-3′ (SEQ ID NO: 34) |
| pAPM-D4 | Lentivector | SFFV promoter expressing puromycin resistance protein |
| miR30- | DNMT3a | and miR30-shRNA target site: 5′- |
| DNMT3a ts3 | knockdown | AGAACTCAAAGAAGAGCCG-3′ (SEQ ID NO: 35) |
| pAPM-D4 | Lentivector | SFFV promoter expressing puromycin resistance protein |
| miR30- | PIWIL2 | and miR30-shRNA target site: 5′- |
| PIWIL2 ts1 | knockdown | CGAACATTGACAACCTGGG-3′ (SEQ ID NO: 36) |
| pAPM-D4 | Lentivector | SFFV promoter expressing puromycin resistance protein |
| miR30- | PIWIL2 | and miR30-shRNA target site: 5′- |
| PIWIL2 ts2 | knockdown | AGCAGACAAGCCTCGACCT-3′ (SEQ ID NO: 37) |
| pAPM-D4 | Lentivector | SFFV promoter expressing puromycin resistance protein |
| miR30- | PIWIL2 | and miR30-shRNA target site: 5′- |
| PIWIL2 ts3 | knockdown | AGATTAGTACTGATTTTCT-3′ (SEQ ID NO: 38) |
| pAPM-D4 | Lentivector | SFFV promoter expressing puromycin resistance protein |
| miR30- | FAM208A | and miR30-shRNA target site: 5′- |
| FAM208A | knockdown | TTCTTCTACTGGTTCCCGG-3′ (SEQ ID NO: 39) |
| ts1 | | |
| pAPM-D4 | Lentivector | SFFV promoter expressing puromycin resistance protein |
| miR30- | FAM208A | and miR30-shRNA target site: 5′- |
| FAM208A | knockdown | TGAATTGCTGTTCTCTCCT-3′ (SEQ ID NO: 40) |
| ts2 | | |
| pAPM-D4 | Lentivector | SFFV promoter expressing puromycin resistance protein |
| miR30- | FAM208A | and miR30-shRNA target site: 5′- |
| FAM208A | knockdown | ATCTTAGCACCAGAATCGT-3′ (SEQ ID NO: 41) |
| ts3 | | |
| pAPM-D4 | Lentivector | SFFV promoter expressing puromycin resistance protein |
| miR30- | MPHOSPH8 | and miR30-shRNA target site: 5′- |
| MPHOSPH8 | knockdown | AAATCTCTTATTTCACCCT-3′ (SEQ ID NO: 42) |
| ts1 | | |
| pAPM-D4 | Lentivector | SFFV promoter expressing puromycin resistance protein |
| miR30- | MPHOSPH8 | and miR30-shRNA target site: 5′- |
| MPHOSPH8 | knockdown | TTGCTTCTGTCTTGATTCC-3′ (SEQ ID NO: 43) |
| ts2 | | |
| pAPM-D4 | Lentivector | SFFV promoter expressing puromycin resistance protein |
| miR30- | MPHOSPH8 | and miR30-shRNA target site: 5′- |
| MPHOSPH8 | knockdown | TTCTCTTCTCTGCTGTCGG-3′ (SEQ ID NO: 44) |
| ts3 | | |
| pAPM-D4 | Lentivector | SFFV promoter expressing puromycin resistance protein |
| miR30- | PPHLN1 | and miR30-shRNA target site: 5′- |
| PPHLN1 ts1 | knockdown | TCATCTGATTTCTCTAGCT-3′ (SEQ ID NO: 45) |
| pAPM-D4 | Lentivector | SFFV promoter expressing puromycin resistance protein |
| miR30- | PPHLN1 | and miR30-shRNA target site: 5′- |
| PPHLN1 ts2 | knockdown | TTCATATTCATATCGTCCC-3′ (SEQ ID NO: 46) |
| pAPM-D4 | Lentivector | SFFV promoter expressing puromycin resistance protein |
| miR30- | PPHLN1 | and miR30-shRNA target site: 5′- |
| PPHLN1 ts3 | knockdown | TGAGTTCTTCAACACACCG-3′ (SEQ ID NO: 47) |
| pAPM-D4 | Lentivector | SFFV promoter expressing puromycin resistance protein |
| miR30- | SUV39h1 | and miR30-shRNA target site: 5′- |
| SUV39h1 ts1 | knockdown | TGAGGATACGCACACACTT-3′ (SEQ ID NO: 48) |
| pAPM-D4 | Lentivector | SFFV promoter expressing puromycin resistance protein |
| miR30- | SUV39h1 | and miR30-shRNA target site: 5′- |
| SUV39h1 ts2 | knockdown | AGAGCAGGTAGGAGCAGGT-3′ (SEQ ID NO: 49) |
| pAPM-D4 | Lentivector | SFFV promoter expressing puromycin resistance protein |
| miR30- | SUV39h1 | and miR30-shRNA target site: 5′- |
| SUV39h1 ts3 | knockdown | CATTCTCTACAGTGATGCG-3′ (SEQ ID NO: 50) |
| pAPM-D4 | Lentivector | SFFV promoter expressing puromycin resistance protein |
| miR30- | SUV39h2 | and miR30-shRNA target site: 5′- |
| SUV39h2 ts1 | knockdown | TCATCAGACTCATAGTCCA-3′ (SEQ ID NO: 51) |
| pAPM-D4 | Lentivector | SFFV promoter expressing puromycin resistance protein |
| miR30- | SUV39h2 | and miR30-shRNA target site: 5′- |
| SUV39h2 ts2 | knockdown | TAAATTTCTTTATCATTGA-3′ (SEQ ID NO: 52) |
| pAPM-D4 | Lentivector | SFFV promoter expressing puromycin resistance protein |
| miR30- | SUV39h2 | and miR30-shRNA target site: 5′- |
| SUV39h2 ts3 | knockdown | ACATTATCAGCTTAACGCT-3′ (SEQ ID NO: 53) |
| pAPM-D4 | Lentivector | SFFV promoter expressing puromycin resistance protein |
| miR30- | SUV39h2 | and miR30-shRNA target site: 5′- |
| MORC2 ts1 | knockdown | TGAGATTGAAGATGATCAC-3′ (SEQ ID NO: 54) |
| pAPM-D4 | Lentivector | SFFV promoter expressing puromycin resistance protein |
| miR30- | MORC2 | and miR30-shRNA target site: 5′- |
| MORC2 ts2 | knockdown | TTTTCCACAGAACTCAGCT-3′ (SEQ ID NO: 55) |
| pAPM-D4 | Lentivector | SFFV promoter expressing puromycin resistance protein |
| miR30- | MORC2 | and miR30-shRNA target site: 5′- |
| MORC2 ts3 | knockdown | TGTCTGTGACAGGTTCCCG-3′ (SEQ ID NO: 56) |
| pAPM- | Lentivector | SFFV promoter expressing puromycin resistance protein |
| miR30- | DCAF1 | and miR30-shRNA target site: 5′- |
| DCAF1 | knockdown | AGCACTTCAGATTATCATCAAT-3′ (SEQ ID NO: 57) |

pAPM-D4 sequence (SEQ ID NO: 10), wherein the position
of insert target sequences is shown with [N..N]
GTCGACGGATCGGGAGATCTCCCGATCCCCTATGGTGCACTCTCAGTACAATCTGCT
CTGATGCCGCATAGTTAAGCCAGTATCTGCTCCCTGCTTGTGTGTTGGAGGTCGCTG
AGTAGTGCGCGAGCAAAATTTAAGCTACAACAAGGCAAGGCTTGACCGACAATTGC
ATGAAGAATCTGCTTAGGGTTAGGCGTTTTGCGCTGCTTCGCGATGTACGGGCCAGA
TATACGCGCTGTGGAATGTGTGTCAGTTAGGGTGTGGAAAGTCCCCAGGCTCCCCA
GCAGGCAGAAGTATGCAAAGCATGCATCTCAATTAGTCAGCAACCAGGTGTGGAAA
GTCCCCAGGCTCCCCAGCAGGCAGAAGTATGCAAAGCATGCATCTCAATTAGTCAG
CAACCATAGTCCCGCCCCTAACTCCGCCCATCCCGCCCCTAACTCCGCCCAGTTCCG
CCCATTCTCCGCCCCATGGCTGACTAATTTTTTTTATTTATGCAGAGGCCGAGGCCG
CCTCTGCCTCTGAGCTATTCCAGAAGTAGTGAGGAGGCTTTTTTGGAGGCCTAGGCT
TTTGCAAAAAGCTTTGACATTGATTATTGACTAGTTATTAATAGTAATCAATTACGG
GGTCATTAGTTCATAGCCCATATATGGAGTTCCGCGTTACATAACTTACGGTAAATG
GCCCGCCTGGCTGACCGCCCAACGACCCCCGCCCATTGACGTCAATAATGACGTAT
GTTCCCATAGTAACGCCAATAGGGACTTTCCATTGACGTCAATGGGTGGAGTATTTA
CGGTAAACTGCCCACTTGGCAGTACATCAAGTGTATCATATGCCAAGTACGCCCCCT
ATTGACGTCAATGACGGTAAATGGCCCGCCTGGCATTATGCCCAGTACATGACCTTA
TGGGACTTTCCTACTTGGCAGTACATCTACGTATTAGTCATCGCTATTACCATGGTG
ATGCGGTTTTGGCAGTACATCAATGGGCGTGGATAGCGGTTTGACTCACGGGGATTT
CCAAGTCTCCACCCCATTGACGTCAATGGGAGTTTGTTTTGGCACCAAAATCAACGG
GACTTTCCAAAATGTCGTAACAACTCCGCCCCATTGACGCAAATGGGCGGTAGGCG
TGTACGGTGGGAGGTCTATATAAGCAGCGCGTTTTGCCTGTACTGGGTCTCTCTGGT
TAGACCAGATCTGAGCCTGGGAGCTCTCTGGCTAACTAGGGAACCCACTGCTTAAG
CCTCAATAAAGCTTGCCTTGAGTGCTTCAAGTAGTGTGTGCCCGTCTGTTGTGTGAC
TCTGGTAACTAGAGATCCCTCAGACCCTTTTAGTCAGTGTGGAAAATCTCTAGCAGT
GGCGCCCGAACAGGGACTTGAAAGCGAAAGGGAAACCAGAGGAGCTCTCTCGACG
CAGGACTCGGCTTGCTGAAGCGCGCACGGCAAGAGGCGAGGGGCGGCGACTGGTG
AGTACGCCAAAAATTTTGACTAGCGGAGGCTAGAAGGAGAGAGATGGGTGCGAGA
GCGTCAGTATTAAGCGGGGGAGAATTAGATCGCGATGGGAAAAAATTCGGTTAAGG
CCAGGGGGAAAGAAAAAATATAAATTAAAACATATAGTATGGGCAAGCAGGGAGC
TAGAACGATTCGCAGTTAATCCTGGCCTGTTAGAAACATCAGAAGGCTGTAGACAA
ATACTGGGACAGCTACAACCATCCCTTCAGACAGGATCAGAAGAACTTAGATCATT
ATATAATACAGTAGCAACCCTCTATTGTGTGCATCAAAGGATAGAGATAAAAGACA
CCAAGGAAGCTTTAGACAAGATAGAGGAAGAGCAAAACAAAAGTAAGACCACCGC
ACAGCAAGCGGCCGGCCGCTGATCTTCAGACCTGGAGGAGGAGATATGAGGGACA
ATTGGAGAAGTGAATTATATAAATATAAAGTAGTAAAAATTGAACCATTAGGAGTA
GCACCCACCAAGGCAAAGAGAAGAGTGGTGCAGAGAGAAAAAAGAGCAGTGGGA
ATAGGAGCTTTGTTCCTTGGGTTCTTGGGAGCAGCAGGAAGCACTATGGGCGCAGC
GTCAATGACGCTGACGGTACAGGCCAGACAATTATTGTCTGGTATAGTGCAGCAGC
AGAACAATTTGCTGAGGGCTATTGAGGCGCAACAGCATCTGTTGCAACTCACAGTC
TGGGGCATCAAGCAGCTCCAGGCAAGAATCCTGGCTGTGGAAAGATACCTAAAGGA
TCAACAGCTCCTGGGGATTTGGGGTTGCTCTGGAAAACTCATTTGCACCACTGCTGT
GCCTTGGAATGCTAGTTGGAGTAATAAATCTCTGGAACAGATTTGGAATCACACGA
CCTGGATGGAGTGGGACAGAGAAATTAACAATTACACAAGCTTAATACACTCCTTA
ATTGAAGAATCGCAAAACCAGCAAGAAAAGAATGAACAAGAATTATTGGAATTAG
ATAAATGGGCAAGTTTGTGGAATTGGTTTAACATAACAAATTGGCTGTGGTATATA
AAATTATTCATAATGATAGTAGGAGGCTTGGTAGGTTTAAGAATAGTTTTTGCTGTA
CTTTCTATAGTGAATAGAGTTAGGCAGGGATATTCACCATTATCGTTTCAGACCCAC
CTCCCAACCCCGAGGGGACCCGACAGGCCCGAAGGAATAGAAGAAGAAGGTGGAG
AGAGAGACAGAGACAGATCCATTCGATTAGTGAACGGATCGGCACTGCGTGCGCCA
ATTCTGCAGACAAATGGCAGTATTCATCCACAATTTTAAAAGAAAAGGGGGGATTG
GGGGGTACAGTGCAGGGGAAAGAATAGTAGACATAATAGCAACAGACATACAAAC
TAAAGAATTACAAAAACAAATTACAAAAATTCAAAATTTTCGGGTTTATTACAGGG
ACAGCAGAGATCCAGTTTGGTTAATTAACTGCAGCCCCGATAAAATAAAAGATTTT
ATTTAGTCTCCAGAAAAAGGGGGGAATGAAAGACCCCACCTGTAGGTTTGGCAAGC
TAGCTGCAGTAACGCCATTTTGCAAGGCATGGAAAAATACCAAACCAAGAATAGAG
AAGTTCAGATCAAGGGCGGGTACATGAAAATAGCTAACGTTGGGCCAAACAGGAT
ATCTGCGGTGAGCAGTTTCGGCCCCGGCCCGGGGCCAAGAACAGATGGTCACCGCA
GTTTCGGCCCCGGCCCGAGGCCAAGAACAGATGGTCCCCAGATATGGCCCAACCCT
CAGCAGTTTCTTAAGACCCATCAGATGTTTCCAGGCTCCCCCAAGGACCTGAAATGA
CCCTGCGCCTTATTTGAATTAACCAATCAGCCTGCTTCTCGCTTCTGTTCGCGCGCTT
CTGCTTCCCGAGCTCTATAAAAGAGCTCACAACCCCTCACTCGGCGCGCCAGTCCTC
CGACAGACTGAGTCGCCCGGGGGTCTAGAACGCGTGCCGCCATGACCGAATACAAA
CCTACCGTGAGGCTGGCTACAAGAGATGATGTCCCAAGGGCTGTGAGAACACTGGC
CGCCGCTTTTGCCGATTACCCTGCCACACGCCACACTGTGGACCCAGATCGGCATAT
CGAGAGAGTGACTGAGCTGCAGGAACTGTTCCTGACCCGAGTGGGCCTGGACATTG
GGAAGGTCTGGGTCGCAGACGATGGAGCAGCTGTGGCTGTCTGGACCACACCAGAG
AGCGTGGAAGCCGGAGCTGTCTTTGCAGAGATCGGCCCTAGAATGGCAGAACTGAG
CGGCTCCAGGCTGGCAGCACAGCAGCAGATGGAGGGACTGCTGGCCCCACACAGG
CCTAAGGAACCAGCATGGTTCCTGGCTACCGTGGGGGTCTCTCCTGACCATCAGGG
CAAAGGACTGGGAAGTGCTGTGGTCCTGCCAGGAGTGGAGGCTGCAGAACGAGCT
GGAGTCCCTGCATTTCTGGAGACCTCTGCTCCACGAAACCTGCCCTTCTATGAACGG
CTGGGCTTTACTGTGACCGCAGATGTGGAGGTCCCCGAAGGACCTAGGACCTGGTG
CATGACACGCAAACCCGGCGCCTGAGCGATCGCCGCGGCCGCCTTCTTAACCCAAC
AGAAGGCTCGAGAAGGTATATTGCTGTTGACAGTGAGCG[N..N]TAGTGAAGCCACA
GATGTA[N..N]TGCCTACTGCCTCGGACTTCAAGGGGCTAGAATTCGGCAGCTGTAGA
TCTTAGCCACTTTTTAAAAGAAAAGGGGGGACTGGAAGGGCTAACTGCATCCGGAC
TGTACTGGGTCTCTCTGGTTAGACCAGATCTGAGCCTGGGAGCTCTCTGGCTAACTA
GGGAACCCACTGCTTAAGCCTCAATAAAGCTTGCCTTGAGTGCTTCAAGTAGTGTGT
GCCCGTCTGTTGTGTGACTCTGGTAACTAGAGATCCCTCAGACCCTTTTAGTCAGTG
TGGAAAATCTCTAGCAGGGCCCGTTTCATGTGAGCAAAAGGCCAGCAAAAGGCCAG
GAACCGTAAAAAGGCCGCGTTGCTGGCGTTTTTCCATAGGCTCCGCCCCCCTGACGA
GCATCACAAAAATCGACGCTCAAGTCAGAGGTGGCGAAACCCGACAGGACTATAA
AGATACCAGGCGTTTCCCCCTGGAAGCTCCCTCGTGCGCTCTCCTGTTCCGACCCTG
CCGCTTACCGGATACCTGTCCGCCTTTCTCCCTTCGGGAAGCGTGGCGCTTTCTCAT
AGCTCACGCTGTAGGTATCTCAGTTCGGTGTAGGTCGTTCGCTCCAAGCTGGGCTGT
GTGCACGAACCCCCCGTTCAGCCCGACCGCTGCGCCTTATCCGGTAACTATCGTCTT
GAGTCCAACCCGGTAAGACACGACTTATCGCCACTGGCAGCAGCCACTGGTAACAG
GATTAGCAGAGCGAGGTATGTAGGCGGTGCTACAGAGTTCTTGAAGTGGTGGCCTA
ACTACGGCTACACTAGAAGAACAGTATTTGGTATCTGCGCTCTGCTGAAGCCAGTTA
CCTTCGGAAAAAGAGTTGGTAGCTCTTGATCCGGCAAACAAACCACCGCTGGTAGC
GGTGGTTTTTTTGTTTGCAAGCAGCAGATTACGCGCAGAAAAAAAGGATCTCAAGA
AGATCCTTTGATCTTTTCTACGGGGTCTGACGCTCAGTGGAACGAAAACTCACGTTA
AGGGATTTTGGTCATGAGATTATCAAAAAGGATCTTCACCTAGATCCTTTTAAATTA
AAAATGAAGTTTTAAATCAATCTAAAGTATATATGAGTAAACTTGGTCTGACAGTTA
CCAATGCTTAATCAGTGAGGCACCTATCTCAGCGATCTGTCTATTTCGTTCATCCAT
AGTTGCCTGACTCCCCGTCGTGTAGATAACTACGATACGGGAGGGCTTACCATCTGG
CCCCAGTGCTGCAATGATACCGCGAGACCCACGCTCACCGGCTCCAGATTTATCAG
CAATAAACCAGCCAGCCGGAAGGGCCGAGCGCAGAAGTGGTCCTGCAACTTTATCC
GCCTCCATCCAGTCTATTAATTGTTGCCGGGAAGCTAGAGTAAGTAGTTCGCCAGTT
AATAGTTTGCGCAACGTTGTTGCCATTGCTACAGGCATCGTGGTGTCACGCTCGTCG
TTTGGTATGGCTTCATTCAGCTCCGGTTCCCAACGATCAAGGCGAGTTACATGATCC
CCCATGTTGTGCAAAAAAGCGGTTAGCTCCTTCGGTCCTCCGATCGTTGTCAGAAGT
AAGTTGGCCGCAGTGTTATCACTCATGGTTATGGCAGCACTGCATAATTCTCTTACT
GTCATGCCATCCGTAAGATGCTTTTCTGTGACTGGTGAGTACTCAACCAAGTCATTC
TGAGAATAGTGTATGCGGCGACCGAGTTGCTCTTGCCCGGCGTCAATACGGGATAA
TACCGCGCCACATAGCAGAACTTTAAAAGTGCTCATCATTGGAAAACGTTCTTCGG
GGCGAAAACTCTCAAGGATCTTACCGCTGTTGAGATCCAGTTCGATGTAACCCACTC
GTGCACCCAACTGATCTTCAGCATCTTTTACTTTCACCAGCGTTTCTGGGTGAGCAA
AAACAGGAAGGCAAAATGCCGCAAAAAAGGGAATAAGGGCGACACGGAAATGTTG
AATACTCATACTCTTCCTTTTTCAATATTATTGAAGCATTTATCAGGGTTATTGTCTC
ATGAGCGGATACATATTTGAATGTATTTAGAAAAATAAACAAATAGGGGTTCCGCG
CACATTTCCCCGAAAAGTGCCACCTGAC
>pAPM-D4_(SEQ ID NO: 11) (+shRNA_targeting_Luciferase_gene)
(targeting sequences in brackets)
GTCGACGGATCGGGAGATCTCCCGATCCCCTATGGTGCACTCTCAGTACAATCTGCT
CTGATGCCGCATAGTTAAGCCAGTATCTGCTCCCTGCTTGTGTGTTGGAGGTCGCTG
AGTAGTGCGCGAGCAAAATTTAAGCTACAACAAGGCAAGGCTTGACCGACAATTGC
ATGAAGAATCTGCTTAGGGTTAGGCGTTTTGCGCTGCTTCGCGATGTACGGGCCAGA
TATACGCGCTGTGGAATGTGTGTCAGTTAGGGTGTGGAAAGTCCCCAGGCTCCCCA
GCAGGCAGAAGTATGCAAAGCATGCATCTCAATTAGTCAGCAACCAGGTGTGGAAA
GTCCCCAGGCTCCCCAGCAGGCAGAAGTATGCAAAGCATGCATCTCAATTAGTCAG
CAACCATAGTCCCGCCCCTAACTCCGCCCATCCCGCCCCTAACTCCGCCCAGTTCCG
CCCATTCTCCGCCCCATGGCTGACTAATTTTTTTTATTTATGCAGAGGCCGAGGCCG
CCTCTGCCTCTGAGCTATTCCAGAAGTAGTGAGGAGGCTTTTTTGGAGGCCTAGGCT
TTTGCAAAAAGCTTTGACATTGATTATTGACTAGTTATTAATAGTAATCAATTACGG
GGTCATTAGTTCATAGCCCATATATGGAGTTCCGCGTTACATAACTTACGGTAAATG
GCCCGCCTGGCTGACCGCCCAACGACCCCCGCCCATTGACGTCAATAATGACGTAT
GTTCCCATAGTAACGCCAATAGGGACTTTCCATTGACGTCAATGGGTGGAGTATTTA
CGGTAAACTGCCCACTTGGCAGTACATCAAGTGTATCATATGCCAAGTACGCCCCCT
ATTGACGTCAATGACGGTAAATGGCCCGCCTGGCATTATGCCCAGTACATGACCTTA
TGGGACTTTCCTACTTGGCAGTACATCTACGTATTAGTCATCGCTATTACCATGGTG
ATGCGGTTTTGGCAGTACATCAATGGGCGTGGATAGCGGTTTGACTCACGGGGATTT
CCAAGTCTCCACCCCATTGACGTCAATGGGAGTTTGTTTTGGCACCAAAATCAACGG
GACTTTCCAAAATGTCGTAACAACTCCGCCCCATTGACGCAAATGGGCGGTAGGCG
TGTACGGTGGGAGGTCTATATAAGCAGCGCGTTTTGCCTGTACTGGGTCTCTCTGGT
TAGACCAGATCTGAGCCTGGGAGCTCTCTGGCTAACTAGGGAACCCACTGCTTAAG
CCTCAATAAAGCTTGCCTTGAGTGCTTCAAGTAGTGTGTGCCCGTCTGTTGTGTGAC
TCTGGTAACTAGAGATCCCTCAGACCCTTTTAGTCAGTGTGGAAAATCTCTAGCAGT
GGCGCCCGAACAGGGACTTGAAAGCGAAAGGGAAACCAGAGGAGCTCTCTCGACG
CAGGACTCGGCTTGCTGAAGCGCGCACGGCAAGAGGCGAGGGGCGGCGACTGGTG
AGTACGCCAAAAATTTTGACTAGCGGAGGCTAGAAGGAGAGAGATGGGTGCGAGA
GCGTCAGTATTAAGCGGGGGAGAATTAGATCGCGATGGGAAAAAATTCGGTTAAGG
CCAGGGGGAAAGAAAAAATATAAATTAAAACATATAGTATGGGCAAGCAGGGAGC
TAGAACGATTCGCAGTTAATCCTGGCCTGTTAGAAACATCAGAAGGCTGTAGACAA
ATACTGGGACAGCTACAACCATCCCTTCAGACAGGATCAGAAGAACTTAGATCATT
ATATAATACAGTAGCAACCCTCTATTGTGTGCATCAAAGGATAGAGATAAAAGACA
CCAAGGAAGCTTTAGACAAGATAGAGGAAGAGCAAAACAAAAGTAAGACCACCGC
ACAGCAAGCGGCCGGCCGCTGATCTTCAGACCTGGAGGAGGAGATATGAGGGACA
ATTGGAGAAGTGAATTATATAAATATAAAGTAGTAAAAATTGAACCATTAGGAGTA
GCACCCACCAAGGCAAAGAGAAGAGTGGTGCAGAGAGAAAAAAGAGCAGTGGGA
ATAGGAGCTTTGTTCCTTGGGTTCTTGGGAGCAGCAGGAAGCACTATGGGCGCAGC
GTCAATGACGCTGACGGTACAGGCCAGACAATTATTGTCTGGTATAGTGCAGCAGC
AGAACAATTTGCTGAGGGCTATTGAGGCGCAACAGCATCTGTTGCAACTCACAGTC
TGGGGCATCAAGCAGCTCCAGGCAAGAATCCTGGCTGTGGAAAGATACCTAAAGGA
TCAACAGCTCCTGGGGATTTGGGGTTGCTCTGGAAAACTCATTTGCACCACTGCTGT
GCCTTGGAATGCTAGTTGGAGTAATAAATCTCTGGAACAGATTTGGAATCACACGA
CCTGGATGGAGTGGGACAGAGAAATTAACAATTACACAAGCTTAATACACTCCTTA
ATTGAAGAATCGCAAAACCAGCAAGAAAAGAATGAACAAGAATTATTGGAATTAG
ATAAATGGGCAAGTTTGTGGAATTGGTTTAACATAACAAATTGGCTGTGGTATATA
AAATTATTCATAATGATAGTAGGAGGCTTGGTAGGTTTAAGAATAGTTTTTGCTGTA
CTTTCTATAGTGAATAGAGTTAGGCAGGGATATTCACCATTATCGTTTCAGACCCAC
CTCCCAACCCCGAGGGGACCCGACAGGCCCGAAGGAATAGAAGAAGAAGGTGGAG
AGAGAGACAGAGACAGATCCATTCGATTAGTGAACGGATCGGCACTGCGTGCGCCA
ATTCTGCAGACAAATGGCAGTATTCATCCACAATTTTAAAAGAAAAGGGGGGATTG
GGGGGTACAGTGCAGGGGAAAGAATAGTAGACATAATAGCAACAGACATACAAAC
TAAAGAATTACAAAAACAAATTACAAAAATTCAAAATTTTCGGGTTTATTACAGGG
ACAGCAGAGATCCAGTTTGGTTAATTAACTGCAGCCCCGATAAAATAAAAGATTTT
ATTTAGTCTCCAGAAAAAGGGGGGAATGAAAGACCCCACCTGTAGGTTTGGCAAGC
TAGCTGCAGTAACGCCATTTTGCAAGGCATGGAAAAATACCAAACCAAGAATAGAG
AAGTTCAGATCAAGGGCGGGTACATGAAAATAGCTAACGTTGGGCCAAACAGGAT
ATCTGCGGTGAGCAGTTTCGGCCCCGGCCCGGGGCCAAGAACAGATGGTCACCGCA
GTTTCGGCCCCGGCCCGAGGCCAAGAACAGATGGTCCCCAGATATGGCCCAACCCT
CAGCAGTTTCTTAAGACCCATCAGATGTTTCCAGGCTCCCCCAAGGACCTGAAATGA
CCCTGCGCCTTATTTGAATTAACCAATCAGCCTGCTTCTCGCTTCTGTTCGCGCGCTT
CTGCTTCCCGAGCTCTATAAAAGAGCTCACAACCCCTCACTCGGCGCGCCAGTCCTC
CGACAGACTGAGTCGCCCGGGGGTCTAGAACGCGTGCCGCCATGACCGAATACAAA
CCTACCGTGAGGCTGGCTACAAGAGATGATGTCCCAAGGGCTGTGAGAACACTGGC
CGCCGCTTTTGCCGATTACCCTGCCACACGCCACACTGTGGACCCAGATCGGCATAT
CGAGAGAGTGACTGAGCTGCAGGAACTGTTCCTGACCCGAGTGGGCCTGGACATTG
GGAAGGTCTGGGTCGCAGACGATGGAGCAGCTGTGGCTGTCTGGACCACACCAGAG
AGCGTGGAAGCCGGAGCTGTCTTTGCAGAGATCGGCCCTAGAATGGCAGAACTGAG
CGGCTCCAGGCTGGCAGCACAGCAGCAGATGGAGGGACTGCTGGCCCCACACAGG
CCTAAGGAACCAGCATGGTTCCTGGCTACCGTGGGGGTCTCTCCTGACCATCAGGG
CAAAGGACTGGGAAGTGCTGTGGTCCTGCCAGGAGTGGAGGCTGCAGAACGAGCT
GGAGTCCCTGCATTTCTGGAGACCTCTGCTCCACGAAACCTGCCCTTCTATGAACGG
CTGGGCTTTACTGTGACCGCAGATGTGGAGGTCCCCGAAGGACCTAGGACCTGGTG
CATGACACGCAAACCCGGCGCCTGAGCGATCGCCGCGGCCGCCTTCTTAACCCAAC
AGAAGGCTCGAGAAGGTATATTGCTGTTGACAGTGAGCG[CACAAACGCTCTCATCG
ACAAG]TAGTGAAGCCACAGATGTA[CTTGTCGATGAGAGCGTTTGTA]TGCCTACTG
CCTCGGACTTCAAGGGGCTAGAATTCGGCAGCTGTAGATCTTAGCCACTTTTTAAAA
GAAAAGGGGGGACTGGAAGGGCTAACTGCATCCGGACTGTACTGGGTCTCTCTGGT
TAGACCAGATCTGAGCCTGGGAGCTCTCTGGCTAACTAGGGAACCCACTGCTTAAG
CCTCAATAAAGCTTGCCTTGAGTGCTTCAAGTAGTGTGTGCCCGTCTGTTGTGTGAC
TCTGGTAACTAGAGATCCCTCAGACCCTTTTAGTCAGTGTGGAAAATCTCTAGCAGG
GCCCGTTTCATGTGAGCAAAAGGCCAGCAAAAGGCCAGGAACCGTAAAAAGGCCG
CGTTGCTGGCGTTTTTCCATAGGCTCCGCCCCCCTGACGAGCATCACAAAAATCGAC
GCTCAAGTCAGAGGTGGCGAAACCCGACAGGACTATAAAGATACCAGGCGTTTCCC
CCTGGAAGCTCCCTCGTGCGCTCTCCTGTTCCGACCCTGCCGCTTACCGGATACCTG
TCCGCCTTTCTCCCTTCGGGAAGCGTGGCGCTTTCTCATAGCTCACGCTGTAGGTAT
CTCAGTTCGGTGTAGGTCGTTCGCTCCAAGCTGGGCTGTGTGCACGAACCCCCCGTT
CAGCCCGACCGCTGCGCCTTATCCGGTAACTATCGTCTTGAGTCCAACCCGGTAAGA
CACGACTTATCGCCACTGGCAGCAGCCACTGGTAACAGGATTAGCAGAGCGAGGTA
TGTAGGCGGTGCTACAGAGTTCTTGAAGTGGTGGCCTAACTACGGCTACACTAGAA
GAACAGTATTTGGTATCTGCGCTCTGCTGAAGCCAGTTACCTTCGGAAAAAGAGTTG
GTAGCTCTTGATCCGGCAAACAAACCACCGCTGGTAGCGGTGGTTTTTTTGTTTGCA
AGCAGCAGATTACGCGCAGAAAAAAAGGATCTCAAGAAGATCCTTTGATCTTTTCT
ACGGGGTCTGACGCTCAGTGGAACGAAAACTCACGTTAAGGGATTTTGGTCATGAG
ATTATCAAAAAGGATCTTCACCTAGATCCTTTTAAATTAAAAATGAAGTTTTAAATC
AATCTAAAGTATATATGAGTAAACTTGGTCTGACAGTTACCAATGCTTAATCAGTGA
GGCACCTATCTCAGCGATCTGTCTATTTCGTTCATCCATAGTTGCCTGACTCCCCGTC
GTGTAGATAACTACGATACGGGAGGGCTTACCATCTGGCCCCAGTGCTGCAATGAT
ACCGCGAGACCCACGCTCACCGGCTCCAGATTTATCAGCAATAAACCAGCCAGCCG
GAAGGGCCGAGCGCAGAAGTGGTCCTGCAACTTTATCCGCCTCCATCCAGTCTATTA
ATTGTTGCCGGGAAGCTAGAGTAAGTAGTTCGCCAGTTAATAGTTTGCGCAACGTTG
TTGCCATTGCTACAGGCATCGTGGTGTCACGCTCGTCGTTTGGTATGGCTTCATTCA
GCTCCGGTTCCCAACGATCAAGGCGAGTTACATGATCCCCCATGTTGTGCAAAAAA
GCGGTTAGCTCCTTCGGTCCTCCGATCGTTGTCAGAAGTAAGTTGGCCGCAGTGTTA
TCACTCATGGTTATGGCAGCACTGCATAATTCTCTTACTGTCATGCCATCCGTAAGA
TGCTTTTCTGTGACTGGTGAGTACTCAACCAAGTCATTCTGAGAATAGTGTATGCGG
CGACCGAGTTGCTCTTGCCCGGCGTCAATACGGGATAATACCGCGCCACATAGCAG
AACTTTAAAAGTGCTCATCATTGGAAAACGTTCTTCGGGGCGAAAACTCTCAAGGA
TCTTACCGCTGTTGAGATCCAGTTCGATGTAACCCACTCGTGCACCCAACTGATCTT
CAGCATCTTTTACTTTCACCAGCGTTTCTGGGTGAGCAAAAACAGGAAGGCAAAAT
GCCGCAAAAAAGGGAATAAGGGCGACACGGAAATGTTGAATACTCATACTCTTCCT
TTTTCAATATTATTGAAGCATTTATCAGGGTTATTGTCTCATGAGCGGATACATATTT
GAATGTATTTAGAAAAATAAACAAATAGGGGTTCCGCGCACATTTCCCCGAAAAGT
GCCACCTGAC

### EQUIVALENTS

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. A construct comprising a nucleic acid encoding a promoter operably linked to transgene encoding a packagable vector RNA, the packagable vector RNA comprising 5′- and 3′-terminal repeats (TRs) that flank:
i. a nucleocapsid protein packaging target site,
ii. a heterologous nucleic acid insert, and
iii. minimal intervening viral sequences.

2. The construct of claim 1, wherein the terminal repeats further flank
i. a REV protein response element (RRE), and
ii. a polypurine tract.

3. The construct of claim 1 or 2, wherein the terminal repeats further flank
i. a sequence encoding a GAG protein.

4. The construct of any one of claims 1 to 3, wherein one or both of the 5′- and 3′-terminal repeats is a lentiviral long terminal repeat.

5. The construct of any one of claims 1 to 3, where one or both of the 5′- and 3′-terminal repeats is a truncated lentiviral long terminal repeat that comprise an R-element that directs reverse transcription and an integrase substrate element that directs integration.

6. The construct of any of claims 1 to 5, wherein the minimal intervening viral sequences are in total up to 350 base pairs in length.

7. A nucleic acid comprising a heterologous nucleic acid insert flanked by terminal repeats (TRs), wherein between a first terminal repeat and the heterologous nucleic acid sequence are present packaging and nuclear export sequences and minimal intervening viral sequences.

8. The nucleic acid of claim 7, wherein the minimal intervening viral sequences are up to a total of 350 base pairs in length.

9. The nucleic acid of any one of claims 1-8, wherein there is an internal promoter operably linked to the heterologous nucleic acid insert located between the nucleocapsid protein packaging target site and the second TR.

10. The nucleic acid of any one of claims 1-9, wherein the internal promoter is optionally spleen focus-forming virus (SFFV) promoter.

11. The nucleic acid of any one of claims 1-10, wherein the 5′-TR is a RNA pol II promoter and comprises a repeat region and a U5 region.

12. The nucleic acid of claim 7 or 8, wherein the 3′-TR is a transcription termination and comprises a repeat region and a U3 region.

13. The nucleic acid of any one of claims 1-12, wherein the packaging sequences comprise a psi (ψ) sequence and a polypurine tract sequence.

14. The nucleic acid of any one of claims 1-13, wherein the order of the packaging sequences is ψ sequence followed by a polypurine tract sequence.

15. The nucleic acid of any one of claims 7-14, wherein the nuclear export sequence comprises a Rev Response Element (RRE).

16. The nucleic acid of any one of claim 2 to 6 or 15, wherein the RRE is located between the ψ sequence and the polypurine tract sequence.

17. The nucleic acid of any one of claims 1-16, wherein the packagable nucleic acid size is 1,900 bases, plus the size of the heterologous nucleic acid insert.

18. The nucleic acid of any one of claims 1-17, wherein the heterologous nucleic acid insert is engineered to express a protein or a functional RNA.

19. The nucleic acid of claim 7, wherein a constitutive promoter is located upstream of the 5′-TR, further wherein the constitutive promoter is CMV or SV40.

20. A plasmid that comprises the nucleic acid of any one of claims 1-19.

21. A method of delivering a plasmid to a cell, the method comprising delivering to the cell a plasmid of any one of claims 1-20.

22. A host cell comprising the nucleic acid of any one of claims 1-21.

23. The host cell of claim 22, wherein the host cell further comprises an RNA polymerase that selectively binds to the 5′-TR of the nucleic acid.

24. The host cell of claim 22 or 23, wherein the host cell further comprises plasmids encoding nucleic acid sequences which facilitate encapsulating and enveloping of the transcribed nucleic acid.

25. The host cell of any one of claims 22-24, wherein the envelope sequence is vesicular stomatitis virus G glycoprotein (VSVG).

26. The host cell of any one of claims 22-25, wherein the encapsulating sequences encode GAG, Pol and Rev proteins.

27. A transcribed nucleic acid encoding a heterologous nucleic acid insert flanked by TRs, wherein between the first TR- and the heterologous nucleic acid sequence, there are sequences that aid in the packaging and nuclear export of the transcribed nucleic acid and minimal intervening viral sequences.

28. A host cell comprising the transcribed nucleic acid of claim 27.

29. A host cell comprising viral particles, wherein the transcribed nucleic acid of claim 27 is within the viral particles.

30. A method of infecting a host cell with the viral particles of claim 29.

31. A method of infecting a subject with the viral particles of claim 29.

32. A composition comprising a plurality of nucleic acids as described in any one of claims 1-19.

33. The composition of claim 32 further comprising a pharmaceutically acceptable carrier.

34. The nucleic acid of any one of claims 1-19, wherein the heterologous nucleic acid insert encodes an shRNA sequence.

35. The nucleic acid of claim 34, wherein there is a selectable marker gene upstream of the shRNA sequence.

36. The nucleic acid of claim 34, wherein there is a reporter gene upstream of the shRNA sequence.

37. The nucleic acid of any one of claims 1-19, wherein there heterologous nucleic acid insert encodes a Cas nuclease gene.

38. The nucleic acid of claim 37, wherein the Cas nuclease is Cas9 nuclease.

39. The nucleic acid of claim 38, wherein the Cas9 nuclease is from Streptococcus pyogenes, Neisseria meningitides, or Campylobacter jejuni.

40. A plasmid that comprises the nucleic acid of any one of claims 34-39.

41. A method of delivering a plasmid to a cell, the method comprising delivering to the cell a plasmid of any one of claims 34-39.

42. A host cell comprising the nucleic acid of any one of claims 34-39.

43. The host cell of claim 42, wherein the host cell further comprises an RNA polymerase that selectively binds to the 5′-TR of the nucleic acid.

44. The host cell of claim 42 or 43, wherein the host cell further comprises plasmids encoding nucleic acid sequences that facilitate packaging of the transcribed nucleic acid.

45. The host cell of any one of claims 42-44, wherein the envelope sequence is vesicular stomatitis virus G glycoprotein (VSVG).

46. The host cell of any one of claims 42-45, wherein the packaging sequences encode GAG, Pol and Rev proteins.

47. A transcribed nucleic acid encoding a heterologous nucleic acid insert flanked by terminal repeats (TR), wherein between the first terminal repeat region and the heterologous nucleic acid sequence, there are sequences that aid in the packaging and nuclear export of the transcribed nucleic acid and minimal intervening viral sequences.

48. A host cell comprising the transcribed nucleic acid of claim 47.

49. A host cell comprising viral particles, wherein the transcribed nucleic acid of claim 47 is within the viral particles.

50. A method of infecting a host cell with the viral particles of claim 49.

51. A method of infecting a subject with the viral particles of claim 49.

52. A composition comprising a plurality of nucleic acids as described in any one of claims 34-39.

53. The composition comprising a nucleic acid as described in any one of claims 34-39 and a pharmaceutically acceptable carrier.

54. The host cell of claim 48 or 49, wherein the host cell is a primary human cell, optionally wherein the host cell is a dendritic cell.

55. A method for efficient gene knockdown, the method comprising infecting target cells with the viral particles of claim 49, wherein the viral particles enclose a transcribed nucleic acid of claim 37 or 38.

56. The method of claim 55, wherein the target cells are primary human cells.

57. The method of claim 56, wherein the primary human cells are dendritic cells.

58. A kit containing a plasmid of claim 20 and/or claim 40.

59. The kit of claim 58, wherein the kit also contains additional plasmids which contain nucleic acids that facilitate packaging and enveloping of the plasmid of claim 20 and/or 40.

60. A construct comprising a promoter operably linked to a nucleic acid encoding a packagable vector RNA, the packagable vector RNA comprising 5′- and 3′-terminal repeats (TRs) that flank:
i. a nucleocapsid protein packaging target site,
ii. a heterologous nucleic acid insert that encodes a microRNA, and
iii. minimal intervening viral sequences.

61. The construct of claim 60, wherein the terminal repeats further flank:
iv. a REV protein response element (RRE), and
v. a polypurine tract.

62. The construct of claim 60 or 61, wherein the terminal repeats further flank:
vi. a sequence encoding a GAG protein.

63. The construct of any one of claims 60-62, wherein the microRNA is processed into a shRNA in a cell.

64. The construct of any one of claims 60-63, wherein the microRNA targets AGO1, AGO2, AGO3, DNMT3A, HDAC1, HP1, SUV39H1, SUV39H2, PIWIL2, TRIM28, SETDB1, FAM208A, MPHOSPH8, PPHLN1, or MORC2.

65. The construct of any one of claims 60-64, wherein one or both of the 5′- and 3′-terminal repeats is a lentiviral long terminal repeat.

66. The construct of any one of claims 60-65, where one or both of the 5′- and 3′-terminal repeats is a truncated lentiviral long terminal repeat that comprise an R-element that directs reverse transcription and an integrase substrate element that directs integration.

67. The construct of any of claims 60-67, wherein the minimal intervening viral sequences are in total up to 350 base pairs in length.

68. A construct comprising a sequence as set forth in SEQ ID NO: 10.

69. A construct comprising a sequence as set forth in SEQ ID NO: 11.

70. A plasmid comprising the construct of any one of claims 60-69.

71. A host cell comprising the plasmid of claim 70.


SEQUENCE LISTING (57 sequences)

SEQ ID NO: 1 - DNA, 6677 bp, Artificial Sequence
  Synthetic Polynucleotide
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaactgcagccccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggtctagaagcgctggatccgtttaaacgcggccgcccagcacagtggctcgagccgcgggttaactggccagaattcacgcgtatcgataccggtggcccctggggccgcgatcgctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcttaatcgcgtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 2 - DNA, 5642 bp, Artificial Sequence
  Synthetic Polynucleotide
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaactgcagccccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggtctagaagcgctggatccgtttaaacgcggccgcccagcacagtggctcgagccgcgggttaactggccagaattcacgcgtatcgataccggtggcccctggggccgcgatcgccagctgtagatcttagccactttttaaaagaaaaggggggactggaagggctaactgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 3 - DNA, 5007 bp, Artificial Sequence
  Synthetic Polynucleotide
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactgacgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctgggggtatacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaactgcagccccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggtctagaagcgctggatccgtttaaacgcggccgcccagcacagtggctcgagccgcgggttaactggccagaattcacgcgtatcgataccggtggcccctggggccgcgatcgccagctgtagatcttagccactttttaaaagaaaaggggggactggaagggctaactgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 4 - DNA, 20 bp, Artificial Sequence
  Synthetic Polynucleotide
gctggaaatgtggaaaggaa

SEQ ID NO: 5 - DNA, 20 bp, Artificial Sequence
  Synthetic Polynucleotide
agtctcttcgccaaacctga

SEQ ID NO: 6 - DNA, 20 bp, Artificial Sequence
  Synthetic Polynucleotide
gcagaggtgaagttcgaagg

SEQ ID NO: 7 - DNA, 20 bp, Artificial Sequence
  Synthetic Polynucleotide
ccaattggtgtgttctgctg

SEQ ID NO: 8 - DNA, 21 bp, Artificial Sequence
  Synthetic Polynucleotide
agggctgcttttaactctggt

SEQ ID NO: 9 - DNA, 21 bp, Artificial Sequence
  Synthetic Polynucleotide
ccccacttgattttggaggga

SEQ ID NO: 10 - DNA, 6275 bp, Artificial Sequence
  Synthetic Polynucleotide
  misc_feature (4140)..(4141): n is a, c, g, or t
  misc_feature (4161)..(4162): n is a, c, g, or t
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaactgcagccccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggtctagaacgcgtgccgccatgaccgaatacaaacctaccgtgaggctggctacaagagatgatgtcccaagggctgtgagaacactggccgccgcttttgccgattaccctgccacacgccacactgtggacccagatcggcatatcgagagagtgactgagctgcaggaactgttcctgacccgagtgggcctggacattgggaaggtctgggtcgcagacgatggagcagctgtggctgtctggaccacaccagagagcgtggaagccggagctgtctttgcagagatcggccctagaatggcagaactgagcggctccaggctggcagcacagcagcagatggagggactgctggccccacacaggcctaaggaaccagcatggttcctggctaccgtgggggtctctcctgaccatcagggcaaaggactgggaagtgctgtggtcctgccaggagtggaggctgcagaacgagctggagtccctgcatttctggagacctctgctccacgaaacctgcccttctatgaacggctgggctttactgtgaccgcagatgtggaggtccccgaaggacctaggacctggtgcatgacacgcaaacccggcgcctgagcgatcgccgcggccgccttcttaacccaacagaaggctcgagaaggtatattgctgttgacagtgagcgnntagtgaagccacagatgtanntgcctactgcctcggacttcaaggggctagaattcggcagctgtagatcttagccactttttaaaagaaaaggggggactggaagggctaactgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 11 - DNA, 6315 bp, Artificial Sequence
  Synthetic Polynucleotide
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaactgcagccccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggtctagaacgcgtgccgccatgaccgaatacaaacctaccgtgaggctggctacaagagatgatgtcccaagggctgtgagaacactggccgccgcttttgccgattaccctgccacacgccacactgtggacccagatcggcatatcgagagagtgactgagctgcaggaactgttcctgacccgagtgggcctggacattgggaaggtctgggtcgcagacgatggagcagctgtggctgtctggaccacaccagagagcgtggaagccggagctgtctttgcagagatcggccctagaatggcagaactgagcggctccaggctggcagcacagcagcagatggagggactgctggccccacacaggcctaaggaaccagcatggttcctggctaccgtgggggtctctcctgaccatcagggcaaaggactgggaagtgctgtggtcctgccaggagtggaggctgcagaacgagctggagtccctgcatttctggagacctctgctccacgaaacctgcccttctatgaacggctgggctttactgtgaccgcagatgtggaggtccccgaaggacctaggacctggtgcatgacacgcaaacccggcgcctgagcgatcgccgcggccgccttcttaacccaacagaaggctcgagaaggtatattgctgttgacagtgagcgcacaaacgctctcatcgacaagtagtgaagccacagatgtacttgtcgatgagagcgtttgtatgcctactgcctcggacttcaaggggctagaattcggcagctgtagatcttagccactttttaaaagaaaaggggggactggaagggctaactgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 12 - DNA, 22 bp, Artificial Sequence
  Synthetic Polynucleotide
tcctgctcaacttcctgtcgag

SEQ ID NO: 13 - DNA, 24 bp, Artificial Sequence
  Synthetic Polynucleotide
cacaggtcaaacctcctaggaatg

SEQ ID NO: 14 - DNA, 21 bp, Artificial Sequence
  Synthetic Polynucleotide
cttgtcgatgagagcgtttgt

SEQ ID NO: 15 - DNA, 19 bp, Artificial Sequence
  Synthetic Polynucleotide
tatgagtcatgcggattcg

SEQ ID NO: 16 - DNA, 19 bp, Artificial Sequence
  Synthetic Polynucleotide
taagaacgggaagaatggg

SEQ ID NO: 17 - DNA, 19 bp, Artificial Sequence
  Synthetic Polynucleotide
ttaatgtagtcatcgctgt

SEQ ID NO: 18 - DNA, 19 bp, Artificial Sequence
  Synthetic Polynucleotide
ttctgcttgaaatactgtg

SEQ ID NO: 19 - DNA, 19 bp, Artificial Sequence
  Synthetic Polynucleotide
tgatatcagagatttctgg

SEQ ID NO: 20 - DNA, 19 bp, Artificial Sequence
  Synthetic Polynucleotide
ttgacattgatcttgaggc

SEQ ID NO: 21 - DNA, 19 bp, Artificial Sequence
  Synthetic Polynucleotide
taatacatctttgtcctgc

SEQ ID NO: 22 - DNA, 19 bp, Artificial Sequence
  Synthetic Polynucleotide
tcatctgcacgcactgcgt

SEQ ID NO: 23 - DNA, 19 bp, Artificial Sequence
  Synthetic Polynucleotide
ttgctaatctcttcttgcc

SEQ ID NO: 24 - DNA, 19 bp, Artificial Sequence
  Synthetic Polynucleotide
tgacttgaacacattgtgt

SEQ ID NO: 25 - DNA, 19 bp, Artificial Sequence
  Synthetic Polynucleotide
tctgaactacaatgtaggt

SEQ ID NO: 26 - DNA, 19 bp, Artificial Sequence
  Synthetic Polynucleotide
tagcttcttgatacatcgt

SEQ ID NO: 27 - DNA, 19 bp, Artificial Sequence
  Synthetic Polynucleotide
ttcgcatgctgactatcag

SEQ ID NO: 28 - DNA, 19 bp, Artificial Sequence
  Synthetic Polynucleotide
acacaatccatcttctcca

SEQ ID NO: 29 - DNA, 19 bp, Artificial Sequence
  Synthetic Polynucleotide
ttgttgtcaaatttcacct

SEQ ID NO: 30 - DNA, 19 bp, Artificial Sequence
  Synthetic Polynucleotide
aaggttgtagtcctcagtg

SEQ ID NO: 31 - DNA, 19 bp, Artificial Sequence
  Synthetic Polynucleotide
tcaataacaataaggttgt

SEQ ID NO: 32 - DNA, 19 bp, Artificial Sequence
  Synthetic Polynucleotide
tgagtagggatcatctcct

SEQ ID NO: 33 - DNA, 19 bp, Artificial Sequence
  Synthetic Polynucleotide
taatctccttgaccttggg

SEQ ID NO: 34 - DNA, 19 bp, Artificial Sequence
  Synthetic Polynucleotide
tatcattcacagtggatgc

SEQ ID NO: 35 - DNA, 19 bp, Artificial Sequence
  Synthetic Polynucleotide
agaactcaaagaagagccg

SEQ ID NO: 36 - DNA, 19 bp, Artificial Sequence
  Synthetic Polynucleotide
cgaacattgacaacctggg

SEQ ID NO: 37 - DNA, 19 bp, Artificial Sequence
  Synthetic Polynucleotide
agcagacaagcctcgacct

SEQ ID NO: 38 - DNA, 19 bp, Artificial Sequence
  Synthetic Polynucleotide
agattagtactgattttct

SEQ ID NO: 39 - DNA, 19 bp, Artificial Sequence
  Synthetic Polynucleotide
ttcttctactggttcccgg

SEQ ID NO: 40 - DNA, 19 bp, Artificial Sequence
  Synthetic Polynucleotide
tgaattgctgttctctcct

SEQ ID NO: 41 - DNA, 19 bp, Artificial Sequence
  Synthetic Polynucleotide
atcttagcaccagaatcgt

SEQ ID NO: 42 - DNA, 19 bp, Artificial Sequence
  Synthetic Polynucleotide
aaatctcttatttcaccct

SEQ ID NO: 43 - DNA, 19 bp, Artificial Sequence
  Synthetic Polynucleotide
ttgcttctgtcttgattcc

SEQ ID NO: 44 - DNA, 19 bp, Artificial Sequence
  Synthetic Polynucleotide
ttctcttctctgctgtcgg

SEQ ID NO: 45 - DNA, 19 bp, Artificial Sequence
  Synthetic Polynucleotide
tcatctgatttctctagct

SEQ ID NO: 46 - DNA, 19 bp, Artificial Sequence
  Synthetic Polynucleotide
ttcatattcatatcgtccc

SEQ ID NO: 47 - DNA, 19 bp, Artificial Sequence
  Synthetic Polynucleotide
tgagttcttcaacacaccg

SEQ ID NO: 48 - DNA, 19 bp, Artificial Sequence
  Synthetic Polynucleotide
tgaggatacgcacacactt

SEQ ID NO: 49 - DNA, 19 bp, Artificial Sequence
  Synthetic Polynucleotide
agagcaggtaggagcaggt

SEQ ID NO: 50 - DNA, 19 bp, Artificial Sequence
  Synthetic Polynucleotide
cattctctacagtgatgcg

SEQ ID NO: 51 - DNA, 19 bp, Artificial Sequence
  Synthetic Polynucleotide
tcatcagactcatagtcca

SEQ ID NO: 52 - DNA, 19 bp, Artificial Sequence
  Synthetic Polynucleotide
taaatttctttatcattga

SEQ ID NO: 53 - DNA, 19 bp, Artificial Sequence
  Synthetic Polynucleotide
acattatcagcttaacgct

SEQ ID NO: 54 - DNA, 19 bp, Artificial Sequence
  Synthetic Polynucleotide
tgagattgaagatgatcac

SEQ ID NO: 55 - DNA, 19 bp, Artificial Sequence
  Synthetic Polynucleotide
ttttccacagaactcagct

SEQ ID NO: 56 - DNA, 19 bp, Artificial Sequence
  Synthetic Polynucleotide
tgtctgtgacaggttcccg

SEQ ID NO: 57 - DNA, 22 bp, Artificial Sequence
  Synthetic Polynucleotide
agcacttcagattatcatcaat